SEQUENCE LISTING (40 sequences)

SEQ ID NO: 1 - DNA, 1878 bp, synthetic construct
  misc_feature 1..1878: note = synthesized coding region
  source 1..1878: mol_type = other DNA
atggataacaaccccaacattaacgagtgcatcccgtacaactgcctctcgaatccagaagaagtgctcttggatggcgagaggatttcgactggcaacagctccatcgacatttccctctccttggttcagttccttgtgtctaacttcgtccctggcggtggcttccttgttggccttatcgacttcgtctggggaattgtccagtgggatgcgtttctggtgcagatagagcagctgatcaacgagaggatcgctgagttcgcgagaaatgctgcaatcgccaaccttgaagggcttggcaacaacttcaacatctacgtggaggcgttcaaggagtgggaagaggaccctaagaatccagcgaccagaacgagggttatagatcggttccgcatcctcgatggccttttggagagggacatcccgagcttccgcatttcgggatttgaggttcctctgctctcagtctacgctcaagctgctaatctgcatctggccatcttgagggattcagtcatctttggcgaacgctggggtcttacgactatcaacgtgaacgagaactacaatcggttgattcggcacatagacgagtatgccgaccactgtgctaacacctacaataggggtctgaacaatctgccaaagtcaacgtatcaagactggataacctacaataggctcagacgggacctcactctcaccgtgctggacatagctgccttctttccgaactacgacaaccggagatatcctattcaacccgttggtcagctcactcgcgaggtctacaccgatcccctcatcaacttcaatccccagctgcaatcggtcgcacagctgcccaccttcaatgtgatggaaaactcagcgatccggaatccccatctgtttgacatacttaacaacctcactatcttcaccgattggttttcagttggacgcaacttctactggggagggcacagagtgatttcaagcctcattggaggagggaacattacatcgcctatctatggaagggaggccaaccaagagccaccaaggtctttcaccttcaacggtccggtgttcagaacacttagcaatcccacattgcgcttgctgcaacagccgtggccagcaccaccattcaatctgaggggagtggagggtgtggagttctcgacgcctacaaactcctttacgtacagaggcagagggacagtggactcactgacagaactcccacctgaggacaactctgttcctccgagggagggctactcgcaccggctttgccatgccaccttcgtccagaggtctggcacgccttttctgaccactggggttgtctttagctggactcaccgctcagcgacgctgaccaacacaatcgacccagagaggatcaatcagatccctctggtgaagggctttcgcgtttggggtggcacaagcgtgatcaccggacctggtttcactggtggggatatcctcagacgcaatacgtttggcgatttcgtgagccttcaagtcaacatcaattccccaatcacccagagatatcggctccgcttcagatacgcctcatccagagacgcaagggtcatcgtccttactggagcagccagcaccggagtcggaggccaagttagcgtcaacatgccgttgcagaaaacgatggaaatcggtgaaaacctcaccagcagaacctttcgctatacagatttcagcaaccctttctccttcagagccaatccggacataatcggcatatccgagcagcccttgttcggtgctgggtccatctcttctggcgagctgtacatcgacaagattgagatcattctcgcagatgcgactctggaggctgaatcggatcttgaaaggtga

SEQ ID NO: 2 - protein, 625 aa, synthetic construct
  REGION 1..625: note = translated from synthesized coding region
MDNNPNINECIPYNCLSNPEEVLLDGERISTGNSSIDISLSLVQFLVSNFVPGGGFLVGLIDFVWGIVQWDAFLVQIEQLINERIAEFARNAAIANLEGLGNNFNIYVEAFKEWEEDPKNPATRTRVIDRFRILDGLLERDIPSFRISGFEVPLLSVYAQAANLHLAILRDSVIFGERWGLTTINVNENYNRLIRHIDEYADHCANTYNRGLNNLPKSTYQDWITYNRLRRDLTLTVLDIAAFFPNYDNRRYPIQPVGQLTREVYTDPLINFNPQLQSVAQLPTFNVMENSAIRNPHLFDILNNLTIFTDWFSVGRNFYWGGHRVISSLIGGGNITSPIYGREANQEPPRSFTFNGPVFRTLSNPTLRLLQQPWPAPPFNLRGVEGVEFSTPTNSFTYRGRGTVDSLTELPPEDNSVPPREGYSHRLCHATFVQRSGTPFLTTGVVFSWTHRSATLTNTIDPERINQIPLVKGFRVWGGTSVITGPGFTGGDILRRNTFGDFVSLQVNINSPITQRYRLRFRYASSRDARVIVLTGAASTGVGGQVSVNMPLQKTMEIGENLTSRTFRYTDFSNPFSFRANPDIIGISEQPLFGAGSISSGELYIDKIEIILADATLEAESDLER

SEQ ID NO: 3 - DNA, 1878 bp, synthetic construct
  misc_feature 1..1878: note = synthesized coding region
  source 1..1878: mol_type = other DNA
atggataacaaccccaacattaacgagtgcatcccgtacaactgcctctcgaatccagaagaagtgctcttggatggcgagaggatttcgactggcaacagctccatcgacatttccctctccttggttcagttccttgtgtctaacttcgtccctggcgccggcttccttgttggccttatcgacttcgtctggggaattgtccagtgggatgcgtttctggtgcagatagagcagctgatcaacgagaggatcgctgagttcgcgagaaatgctgcaatcgccaaccttgaagggcttggcaacaacttcaacatctacgtggaggcgttcaaggagtgggaagaggaccctaagaatccagcgaccagaacgagggttatagatcggttccgcatcctcgatggccttttggagagggacatcccgagcttccgcatttcgggatttgaggttcctctgctctcagtctacgctcaagctgctaatctgcatctggccatcttgagggattcagtcatctttggcgaacgctggggtcttacgactatcaacgtgaacgagaactacaatcggttgattcggcacatagacgagtatgccgaccactgtgctaacacctacaataggggtctgaacaatctgccaaagtcaacgtatcaagactggataacctacaataggctcagacgggacctcactctcaccgtgctggacatagctgccttctttccgaactacgacaaccggagatatcctattcaacccgttggtcagctcactcgcgaggtctacaccgatcccctcatcaacttcaatccccagctgcaatcggtcgcacagctgcccaccttcaatgtgatggaaaactcagcgatccggaatccccatctgtttgacatacttaacaacctcactatcttcaccgattggttttcagttggacgcaacttctactggggagggcacagagtgatttcaagcctcattggaggagggaacattacatcgcctatctatggaagggaggccaaccaagagccaccaaggtctttcaccttcaacggtccggtgttcagaacacttagcaatcccacattgcgcttgctgcaacagccgtggccagcaccaccattcaatctgaggggagtggagggtgtggagttctcgacgcctacaaactcctttacgtacagaggcagagggacagtggactcactgacagaactcccacctgaggacaactctgttcctccgagggagggctactcgcaccggctttgccatgccaccttcgtccagaggtctggcacgccttttctgaccactggggttgtctttagctggactcaccgctcagcgacgctgaccaacacaatcgacccagagaggatcaatcagatccctctggtgaagggctttcgcgtttggggtggcacaagcgtgatcaccggacctggtttcactggtggggatatcctcagacgcaatacgtttggcgatttcgtgagccttcaagtcaacatcaattccccaatcacccagagatatcggctccgcttcagatacgcctcatccagagacgcaagggtcatcgtccttactggagcagccagcaccggagtcggaggccaagttagcgtcaacatgccgttgcagaaaacgatggaaatcggtgaaaacctcaccagcagaacctttcgctatacagatttcagcaaccctttctccttcagagccaatccggacataatcggcatatccgagcagcccttgttcggtgctgggtccatctcttctggcgagctgtacatcgacaagattgagatcattctcgcagatgcgactctggaggctgaatcggatcttgaaaggtga

SEQ ID NO: 4 - protein, 625 aa, synthetic construct
  REGION 1..625: note = translated from synthesized coding region
MDNNPNINECIPYNCLSNPEEVLLDGERISTGNSSIDISLSLVQFLVSNFVPGAGFLVGLIDFVWGIVQWDAFLVQIEQLINERIAEFARNAAIANLEGLGNNFNIYVEAFKEWEEDPKNPATRTRVIDRFRILDGLLERDIPSFRISGFEVPLLSVYAQAANLHLAILRDSVIFGERWGLTTINVNENYNRLIRHIDEYADHCANTYNRGLNNLPKSTYQDWITYNRLRRDLTLTVLDIAAFFPNYDNRRYPIQPVGQLTREVYTDPLINFNPQLQSVAQLPTFNVMENSAIRNPHLFDILNNLTIFTDWFSVGRNFYWGGHRVISSLIGGGNITSPIYGREANQEPPRSFTFNGPVFRTLSNPTLRLLQQPWPAPPFNLRGVEGVEFSTPTNSFTYRGRGTVDSLTELPPEDNSVPPREGYSHRLCHATFVQRSGTPFLTTGVVFSWTHRSATLTNTIDPERINQIPLVKGFRVWGGTSVITGPGFTGGDILRRNTFGDFVSLQVNINSPITQRYRLRFRYASSRDARVIVLTGAASTGVGGQVSVNMPLQKTMEIGENLTSRTFRYTDFSNPFSFRANPDIIGISEQPLFGAGSISSGELYIDKIEIILADATLEAESDLER

SEQ ID NO: 5 - DNA, 1878 bp, synthetic construct
  misc_feature 1..1878: note = synthesized coding region
  source 1..1878: mol_type = other DNA
atggataacaaccccaacattaacgagtgcatcccgtacaactgcctctcgaatccagaagaagtgctcttggatggcgagaggatttcgactggcaacagctccatcgacatttccctctccttggttcagttccttgtgtctaacttcgtccctggcggtggcttcgccgttggccttatcgacttcgtctggggaattgtccagtgggatgcgtttctggtgcagatagagcagctgatcaacgagaggatcgctgagttcgcgagaaatgctgcaatcgccaaccttgaagggcttggcaacaacttcaacatctacgtggaggcgttcaaggagtgggaagaggaccctaagaatccagcgaccagaacgagggttatagatcggttccgcatcctcgatggccttttggagagggacatcccgagcttccgcatttcgggatttgaggttcctctgctctcagtctacgctcaagctgctaatctgcatctggccatcttgagggattcagtcatctttggcgaacgctggggtcttacgactatcaacgtgaacgagaactacaatcggttgattcggcacatagacgagtatgccgaccactgtgctaacacctacaataggggtctgaacaatctgccaaagtcaacgtatcaagactggataacctacaataggctcagacgggacctcactctcaccgtgctggacatagctgccttctttccgaactacgacaaccggagatatcctattcaacccgttggtcagctcactcgcgaggtctacaccgatcccctcatcaacttcaatccccagctgcaatcggtcgcacagctgcccaccttcaatgtgatggaaaactcagcgatccggaatccccatctgtttgacatacttaacaacctcactatcttcaccgattggttttcagttggacgcaacttctactggggagggcacagagtgatttcaagcctcattggaggagggaacattacatcgcctatctatggaagggaggccaaccaagagccaccaaggtctttcaccttcaacggtccggtgttcagaacacttagcaatcccacattgcgcttgctgcaacagccgtggccagcaccaccattcaatctgaggggagtggagggtgtggagttctcgacgcctacaaactcctttacgtacagaggcagagggacagtggactcactgacagaactcccacctgaggacaactctgttcctccgagggagggctactcgcaccggctttgccatgccaccttcgtccagaggtctggcacgccttttctgaccactggggttgtctttagctggactcaccgctcagcgacgctgaccaacacaatcgacccagagaggatcaatcagatccctctggtgaagggctttcgcgtttggggtggcacaagcgtgatcaccggacctggtttcactggtggggatatcctcagacgcaatacgtttggcgatttcgtgagccttcaagtcaacatcaattccccaatcacccagagatatcggctccgcttcagatacgcctcatccagagacgcaagggtcatcgtccttactggagcagccagcaccggagtcggaggccaagttagcgtcaacatgccgttgcagaaaacgatggaaatcggtgaaaacctcaccagcagaacctttcgctatacagatttcagcaaccctttctccttcagagccaatccggacataatcggcatatccgagcagcccttgttcggtgctgggtccatctcttctggcgagctgtacatcgacaagattgagatcattctcgcagatgcgactctggaggctgaatcggatcttgaaaggtga

SEQ ID NO: 6 - protein, 625 aa, synthetic construct
  REGION 1..625: note = translated from synthesized coding region
MDNNPNINECIPYNCLSNPEEVLLDGERISTGNSSIDISLSLVQFLVSNFVPGGGFAVGLIDFVWGIVQWDAFLVQIEQLINERIAEFARNAAIANLEGLGNNFNIYVEAFKEWEEDPKNPATRTRVIDRFRILDGLLERDIPSFRISGFEVPLLSVYAQAANLHLAILRDSVIFGERWGLTTINVNENYNRLIRHIDEYADHCANTYNRGLNNLPKSTYQDWITYNRLRRDLTLTVLDIAAFFPNYDNRRYPIQPVGQLTREVYTDPLINFNPQLQSVAQLPTFNVMENSAIRNPHLFDILNNLTIFTDWFSVGRNFYWGGHRVISSLIGGGNITSPIYGREANQEPPRSFTFNGPVFRTLSNPTLRLLQQPWPAPPFNLRGVEGVEFSTPTNSFTYRGRGTVDSLTELPPEDNSVPPREGYSHRLCHATFVQRSGTPFLTTGVVFSWTHRSATLTNTIDPERINQIPLVKGFRVWGGTSVITGPGFTGGDILRRNTFGDFVSLQVNINSPITQRYRLRFRYASSRDARVIVLTGAASTGVGGQVSVNMPLQKTMEIGENLTSRTFRYTDFSNPFSFRANPDIIGISEQPLFGAGSISSGELYIDKIEIILADATLEAESDLER

SEQ ID NO: 7 - DNA, 1878 bp, synthetic construct
  misc_feature 1..1878: note = synthesized coding region
  source 1..1878: mol_type = other DNA
atggataacaaccccaacattaacgagtgcatcccgtacaactgcctctcgaatccagaagaagtgctcttggatggcgagaggatttcgactggcaacagctccatcgacatttccctctccttggttcagttccttgtgtctaacttcgtccctggcggtggcttcatggttggccttatcgacttcgtctggggaattgtccagtgggatgcgtttctggtgcagatagagcagctgatcaacgagaggatcgctgagttcgcgagaaatgctgcaatcgccaaccttgaagggcttggcaacaacttcaacatctacgtggaggcgttcaaggagtgggaagaggaccctaagaatccagcgaccagaacgagggttatagatcggttccgcatcctcgatggccttttggagagggacatcccgagcttccgcatttcgggatttgaggttcctctgctctcagtctacgctcaagctgctaatctgcatctggccatcttgagggattcagtcatctttggcgaacgctggggtcttacgactatcaacgtgaacgagaactacaatcggttgattcggcacatagacgagtatgccgaccactgtgctaacacctacaataggggtctgaacaatctgccaaagtcaacgtatcaagactggataacctacaataggctcagacgggacctcactctcaccgtgctggacatagctgccttctttccgaactacgacaaccggagatatcctattcaacccgttggtcagctcactcgcgaggtctacaccgatcccctcatcaacttcaatccccagctgcaatcggtcgcacagctgcccaccttcaatgtgatggaaaactcagcgatccggaatccccatctgtttgacatacttaacaacctcactatcttcaccgattggttttcagttggacgcaacttctactggggagggcacagagtgatttcaagcctcattggaggagggaacattacatcgcctatctatggaagggaggccaaccaagagccaccaaggtctttcaccttcaacggtccggtgttcagaacacttagcaatcccacattgcgcttgctgcaacagccgtggccagcaccaccattcaatctgaggggagtggagggtgtggagttctcgacgcctacaaactcctttacgtacagaggcagagggacagtggactcactgacagaactcccacctgaggacaactctgttcctccgagggagggctactcgcaccggctttgccatgccaccttcgtccagaggtctggcacgccttttctgaccactggggttgtctttagctggactcaccgctcagcgacgctgaccaacacaatcgacccagagaggatcaatcagatccctctggtgaagggctttcgcgtttggggtggcacaagcgtgatcaccggacctggtttcactggtggggatatcctcagacgcaatacgtttggcgatttcgtgagccttcaagtcaacatcaattccccaatcacccagagatatcggctccgcttcagatacgcctcatccagagacgcaagggtcatcgtccttactggagcagccagcaccggagtcggaggccaagttagcgtcaacatgccgttgcagaaaacgatggaaatcggtgaaaacctcaccagcagaacctttcgctatacagatttcagcaaccctttctccttcagagccaatccggacataatcggcatatccgagcagcccttgttcggtgctgggtccatctcttctggcgagctgtacatcgacaagattgagatcattctcgcagatgcgactctggaggctgaatcggatcttgaaaggtga

SEQ ID NO: 8 - protein, 625 aa, synthetic construct
  REGION 1..625: note = translated from synthesized coding region
MDNNPNINECIPYNCLSNPEEVLLDGERISTGNSSIDISLSLVQFLVSNFVPGGGFMVGLIDFVWGIVQWDAFLVQIEQLINERIAEFARNAAIANLEGLGNNFNIYVEAFKEWEEDPKNPATRTRVIDRFRILDGLLERDIPSFRISGFEVPLLSVYAQAANLHLAILRDSVIFGERWGLTTINVNENYNRLIRHIDEYADHCANTYNRGLNNLPKSTYQDWITYNRLRRDLTLTVLDIAAFFPNYDNRRYPIQPVGQLTREVYTDPLINFNPQLQSVAQLPTFNVMENSAIRNPHLFDILNNLTIFTDWFSVGRNFYWGGHRVISSLIGGGNITSPIYGREANQEPPRSFTFNGPVFRTLSNPTLRLLQQPWPAPPFNLRGVEGVEFSTPTNSFTYRGRGTVDSLTELPPEDNSVPPREGYSHRLCHATFVQRSGTPFLTTGVVFSWTHRSATLTNTIDPERINQIPLVKGFRVWGGTSVITGPGFTGGDILRRNTFGDFVSLQVNINSPITQRYRLRFRYASSRDARVIVLTGAASTGVGGQVSVNMPLQKTMEIGENLTSRTFRYTDFSNPFSFRANPDIIGISEQPLFGAGSISSGELYIDKIEIILADATLEAESDLER

SEQ ID NO: 9 - DNA, 1887 bp, Bacillus thuringiensis
  source 1..1887: mol_type = other DNA
atggataacaaccccaacattaacgagtgcatcccgtacaactgcctctcgaatccagaagaagtgctcttggatggcgagaggatttcgactggcaacagctccatcgacatttccctctccttggttcagttccttgtgtctaacttcgtccctggcggtggcttccttgttggccttatcgacttcgtctggggaattgtcggaccctcccagtgggatgcgtttctggtgcagatagagcagctgatcaacgagaggatcgctgagttcgcgagaaatgctgcaatcgccaaccttgaagggcttggcaacaacttcaacatctacgtggaggcgttcaaggagtgggaagaggaccctaagaatccagcgaccagaacgagggttatagatcggttccgcatcctcgatggccttttggagagggacatcccgagcttccgcatttcgggatttgaggttcctctgctctcagtctacgctcaagctgctaatctgcatctggccatcttgagggattcagtcatctttggcgaacgctggggtcttacgactatcaacgtgaacgagaactacaatcggttgattcggcacatagacgagtatgccgaccactgtgctaacacctacaataggggtctgaacaatctgccaaagtcaacgtatcaagactggataacctacaataggctcagacgggacctcactctcaccgtgctggacatagctgccttctttccgaactacgacaaccggagatatcctattcaacccgttggtcagctcactcgcgaggtctacaccgatcccctcatcaacttcaatccccagctgcaatcggtcgcacagctgcccaccttcaatgtgatggaaaactcagcgatccggaatccccatctgtttgacatacttaacaacctcactatcttcaccgattggttttcagttggacgcaacttctactggggagggcacagagtgatttcaagcctcattggaggagggaacattacatcgcctatctatggaagggaggccaaccaagagccaccaaggtctttcaccttcaacggtccggtgttcagaacacttagcaatcccacattgcgcttgctgcaacagccgtggccagcaccaccattcaatctgaggggagtggagggtgtggagttctcgacgcctacaaactcctttacgtacagaggcagagggacagtggactcactgacagaactcccacctgaggacaactctgttcctccgagggagggctactcgcaccggctttgccatgccaccttcgtccagaggtctggcacgccttttctgaccactggggttgtctttagctggactcaccgctcagcgacgctgaccaacacaatcgacccagagaggatcaatcagatccctctggtgaagggctttcgcgtttggggtggcacaagcgtgatcaccggacctggtttcactggtggggatatcctcagacgcaatacgtttggcgatttcgtgagccttcaagtcaacatcaattccccaatcacccagagatatcggctccgcttcagatacgcctcatccagagacgcaagggtcatcgtccttactggagcagccagcaccggagtcggaggccaagttagcgtcaacatgccgttgcagaaaacgatggaaatcggtgaaaacctcaccagcagaacctttcgctatacagatttcagcaaccctttctccttcagagccaatccggacataatcggcatatccgagcagcccttgttcggtgctgggtccatctcttctggcgagctgtacatcgacaagattgagatcattctcgcagatgcgactctggaggctgaatcggatcttgaaaggtga

SEQ ID NO: 10 - protein, 628 aa, Bacillus thuringiensis
MDNNPNINECIPYNCLSNPEEVLLDGERISTGNSSIDISLSLVQFLVSNFVPGGGFLVGLIDFVWGIVGPSQWDAFLVQIEQLINERIAEFARNAAIANLEGLGNNFNIYVEAFKEWEEDPKNPATRTRVIDRFRILDGLLERDIPSFRISGFEVPLLSVYAQAANLHLAILRDSVIFGERWGLTTINVNENYNRLIRHIDEYADHCANTYNRGLNNLPKSTYQDWITYNRLRRDLTLTVLDIAAFFPNYDNRRYPIQPVGQLTREVYTDPLINFNPQLQSVAQLPTFNVMENSAIRNPHLFDILNNLTIFTDWFSVGRNFYWGGHRVISSLIGGGNITSPIYGREANQEPPRSFTFNGPVFRTLSNPTLRLLQQPWPAPPFNLRGVEGVEFSTPTNSFTYRGRGTVDSLTELPPEDNSVPPREGYSHRLCHATFVQRSGTPFLTTGVVFSWTHRSATLTNTIDPERINQIPLVKGFRVWGGTSVITGPGFTGGDILRRNTFGDFVSLQVNINSPITQRYRLRFRYASSRDARVIVLTGAASTGVGGQVSVNMPLQKTMEIGENLTSRTFRYTDFSNPFSFRANPDIIGISEQPLFGAGSISSGELYIDKIEIILADATLEAESDLER

SEQ ID NO: 11 - DNA, 1887 bp, synthetic construct
  misc_feature 1..1887: note = synthesized coding region
  source 1..1887: mol_type = other DNA
atggataacaaccccaacattaacgagtgcatcccgtacaactgcctctcgaatccagaagaagtgctcttggatggcgagaggatttcgactggcaacagctccatcgacatttccctctccttggttcagttccttgtgtctaacttcgtccctggcgccggcttccttgttggccttatcgacttcgtctggggaattgtcggaccctcccagtgggatgcgtttctggtgcagatagagcagctgatcaacgagaggatcgctgagttcgcgagaaatgctgcaatcgccaaccttgaagggcttggcaacaacttcaacatctacgtggaggcgttcaaggagtgggaagaggaccctaagaatccagcgaccagaacgagggttatagatcggttccgcatcctcgatggccttttggagagggacatcccgagcttccgcatttcgggatttgaggttcctctgctctcagtctacgctcaagctgctaatctgcatctggccatcttgagggattcagtcatctttggcgaacgctggggtcttacgactatcaacgtgaacgagaactacaatcggttgattcggcacatagacgagtatgccgaccactgtgctaacacctacaataggggtctgaacaatctgccaaagtcaacgtatcaagactggataacctacaataggctcagacgggacctcactctcaccgtgctggacatagctgccttctttccgaactacgacaaccggagatatcctattcaacccgttggtcagctcactcgcgaggtctacaccgatcccctcatcaacttcaatccccagctgcaatcggtcgcacagctgcccaccttcaatgtgatggaaaactcagcgatccggaatccccatctgtttgacatacttaacaacctcactatcttcaccgattggttttcagttggacgcaacttctactggggagggcacagagtgatttcaagcctcattggaggagggaacattacatcgcctatctatggaagggaggccaaccaagagccaccaaggtctttcaccttcaacggtccggtgttcagaacacttagcaatcccacattgcgcttgctgcaacagccgtggccagcaccaccattcaatctgaggggagtggagggtgtggagttctcgacgcctacaaactcctttacgtacagaggcagagggacagtggactcactgacagaactcccacctgaggacaactctgttcctccgagggagggctactcgcaccggctttgccatgccaccttcgtccagaggtctggcacgccttttctgaccactggggttgtctttagctggactcaccgctcagcgacgctgaccaacacaatcgacccagagaggatcaatcagatccctctggtgaagggctttcgcgtttggggtggcacaagcgtgatcaccggacctggtttcactggtggggatatcctcagacgcaatacgtttggcgatttcgtgagccttcaagtcaacatcaattccccaatcacccagagatatcggctccgcttcagatacgcctcatccagagacgcaagggtcatcgtccttactggagcagccagcaccggagtcggaggccaagttagcgtcaacatgccgttgcagaaaacgatggaaatcggtgaaaacctcaccagcagaacctttcgctatacagatttcagcaaccctttctccttcagagccaatccggacataatcggcatatccgagcagcccttgttcggtgctgggtccatctcttctggcgagctgtacatcgacaagattgagatcattctcgcagatgcgactctggaggctgaatcggatcttgaaaggtga

SEQ ID NO: 12 - protein, 628 aa, synthetic construct
  REGION 1..628: note = translated from synthesized coding region
MDNNPNINECIPYNCLSNPEEVLLDGERISTGNSSIDISLSLVQFLVSNFVPGAGFLVGLIDFVWGIVGPSQWDAFLVQIEQLINERIAEFARNAAIANLEGLGNNFNIYVEAFKEWEEDPKNPATRTRVIDRFRILDGLLERDIPSFRISGFEVPLLSVYAQAANLHLAILRDSVIFGERWGLTTINVNENYNRLIRHIDEYADHCANTYNRGLNNLPKSTYQDWITYNRLRRDLTLTVLDIAAFFPNYDNRRYPIQPVGQLTREVYTDPLINFNPQLQSVAQLPTFNVMENSAIRNPHLFDILNNLTIFTDWFSVGRNFYWGGHRVISSLIGGGNITSPIYGREANQEPPRSFTFNGPVFRTLSNPTLRLLQQPWPAPPFNLRGVEGVEFSTPTNSFTYRGRGTVDSLTELPPEDNSVPPREGYSHRLCHATFVQRSGTPFLTTGVVFSWTHRSATLTNTIDPERINQIPLVKGFRVWGGTSVITGPGFTGGDILRRNTFGDFVSLQVNINSPITQRYRLRFRYASSRDARVIVLTGAASTGVGGQVSVNMPLQKTMEIGENLTSRTFRYTDFSNPFSFRANPDIIGISEQPLFGAGSISSGELYIDKIEIILADATLEAESDLER

SEQ ID NO: 13 - DNA, 1887 bp, synthetic construct
  misc_feature 1..1887: note = synthesized coding region
  source 1..1887: mol_type = other DNA
atggataacaaccccaacattaacgagtgcatcccgtacaactgcctctcgaatccagaagaagtgctcttggatggcgagaggatttcgactggcaacagctccatcgacatttccctctccttggttcagttccttgtgtctaacttcgtccctggcggtggcttcatggttggccttatcgacttcgtctggggaattgtcggaccctcccagtgggatgcgtttctggtgcagatagagcagctgatcaacgagaggatcgctgagttcgcgagaaatgctgcaatcgccaaccttgaagggcttggcaacaacttcaacatctacgtggaggcgttcaaggagtgggaagaggaccctaagaatccagcgaccagaacgagggttatagatcggttccgcatcctcgatggccttttggagagggacatcccgagcttccgcatttcgggatttgaggttcctctgctctcagtctacgctcaagctgctaatctgcatctggccatcttgagggattcagtcatctttggcgaacgctggggtcttacgactatcaacgtgaacgagaactacaatcggttgattcggcacatagacgagtatgccgaccactgtgctaacacctacaataggggtctgaacaatctgccaaagtcaacgtatcaagactggataacctacaataggctcagacgggacctcactctcaccgtgctggacatagctgccttctttccgaactacgacaaccggagatatcctattcaacccgttggtcagctcactcgcgaggtctacaccgatcccctcatcaacttcaatccccagctgcaatcggtcgcacagctgcccaccttcaatgtgatggaaaactcagcgatccggaatccccatctgtttgacatacttaacaacctcactatcttcaccgattggttttcagttggacgcaacttctactggggagggcacagagtgatttcaagcctcattggaggagggaacattacatcgcctatctatggaagggaggccaaccaagagccaccaaggtctttcaccttcaacggtccggtgttcagaacacttagcaatcccacattgcgcttgctgcaacagccgtggccagcaccaccattcaatctgaggggagtggagggtgtggagttctcgacgcctacaaactcctttacgtacagaggcagagggacagtggactcactgacagaactcccacctgaggacaactctgttcctccgagggagggctactcgcaccggctttgccatgccaccttcgtccagaggtctggcacgccttttctgaccactggggttgtctttagctggactcaccgctcagcgacgctgaccaacacaatcgacccagagaggatcaatcagatccctctggtgaagggctttcgcgtttggggtggcacaagcgtgatcaccggacctggtttcactggtggggatatcctcagacgcaatacgtttggcgatttcgtgagccttcaagtcaacatcaattccccaatcacccagagatatcggctccgcttcagatacgcctcatccagagacgcaagggtcatcgtccttactggagcagccagcaccggagtcggaggccaagttagcgtcaacatgccgttgcagaaaacgatggaaatcggtgaaaacctcaccagcagaacctttcgctatacagatttcagcaaccctttctccttcagagccaatccggacataatcggcatatccgagcagcccttgttcggtgctgggtccatctcttctggcgagctgtacatcgacaagattgagatcattctcgcagatgcgactctggaggctgaatcggatcttgaaaggtga

SEQ ID NO: 14 - protein, 628 aa, synthetic construct
  REGION 1..628: note = translated from synthesized coding region
MDNNPNINECIPYNCLSNPEEVLLDGERISTGNSSIDISLSLVQFLVSNFVPGGGFMVGLIDFVWGIVGPSQWDAFLVQIEQLINERIAEFARNAAIANLEGLGNNFNIYVEAFKEWEEDPKNPATRTRVIDRFRILDGLLERDIPSFRISGFEVPLLSVYAQAANLHLAILRDSVIFGERWGLTTINVNENYNRLIRHIDEYADHCANTYNRGLNNLPKSTYQDWITYNRLRRDLTLTVLDIAAFFPNYDNRRYPIQPVGQLTREVYTDPLINFNPQLQSVAQLPTFNVMENSAIRNPHLFDILNNLTIFTDWFSVGRNFYWGGHRVISSLIGGGNITSPIYGREANQEPPRSFTFNGPVFRTLSNPTLRLLQQPWPAPPFNLRGVEGVEFSTPTNSFTYRGRGTVDSLTELPPEDNSVPPREGYSHRLCHATFVQRSGTPFLTTGVVFSWTHRSATLTNTIDPERINQIPLVKGFRVWGGTSVITGPGFTGGDILRRNTFGDFVSLQVNINSPITQRYRLRFRYASSRDARVIVLTGAASTGVGGQVSVNMPLQKTMEIGENLTSRTFRYTDFSNPFSFRANPDIIGISEQPLFGAGSISSGELYIDKIEIILADATLEAESDLER

SEQ ID NO: 15 - DNA, 1887 bp, synthetic construct
  misc_feature 1..1887: note = synthesized coding region
  source 1..1887: mol_type = other DNA
atggataacaaccccaacattaacgagtgcatcccgtacaactgcctctcgaatccagaagaagtgctcttggatggcgagaggatttcgactggcaacagctccatcgacatttccctctccttggttcagttccttgtgtctaacttcgtccctggcggtggcttcgccgttggccttatcgacttcgtctggggaattgtcggaccctcccagtgggatgcgtttctggtgcagatagagcagctgatcaacgagaggatcgctgagttcgcgagaaatgctgcaatcgccaaccttgaagggcttggcaacaacttcaacatctacgtggaggcgttcaaggagtgggaagaggaccctaagaatccagcgaccagaacgagggttatagatcggttccgcatcctcgatggccttttggagagggacatcccgagcttccgcatttcgggatttgaggttcctctgctctcagtctacgctcaagctgctaatctgcatctggccatcttgagggattcagtcatctttggcgaacgctggggtcttacgactatcaacgtgaacgagaactacaatcggttgattcggcacatagacgagtatgccgaccactgtgctaacacctacaataggggtctgaacaatctgccaaagtcaacgtatcaagactggataacctacaataggctcagacgggacctcactctcaccgtgctggacatagctgccttctttccgaactacgacaaccggagatatcctattcaacccgttggtcagctcactcgcgaggtctacaccgatcccctcatcaacttcaatccccagctgcaatcggtcgcacagctgcccaccttcaatgtgatggaaaactcagcgatccggaatccccatctgtttgacatacttaacaacctcactatcttcaccgattggttttcagttggacgcaacttctactggggagggcacagagtgatttcaagcctcattggaggagggaacattacatcgcctatctatggaagggaggccaaccaagagccaccaaggtctttcaccttcaacggtccggtgttcagaacacttagcaatcccacattgcgcttgctgcaacagccgtggccagcaccaccattcaatctgaggggagtggagggtgtggagttctcgacgcctacaaactcctttacgtacagaggcagagggacagtggactcactgacagaactcccacctgaggacaactctgttcctccgagggagggctactcgcaccggctttgccatgccaccttcgtccagaggtctggcacgccttttctgaccactggggttgtctttagctggactcaccgctcagcgacgctgaccaacacaatcgacccagagaggatcaatcagatccctctggtgaagggctttcgcgtttggggtggcacaagcgtgatcaccggacctggtttcactggtggggatatcctcagacgcaatacgtttggcgatttcgtgagccttcaagtcaacatcaattccccaatcacccagagatatcggctccgcttcagatacgcctcatccagagacgcaagggtcatcgtccttactggagcagccagcaccggagtcggaggccaagttagcgtcaacatgccgttgcagaaaacgatggaaatcggtgaaaacctcaccagcagaacctttcgctatacagatttcagcaaccctttctccttcagagccaatccggacataatcggcatatccgagcagcccttgttcggtgctgggtccatctcttctggcgagctgtacatcgacaagattgagatcattctcgcagatgcgactctggaggctgaatcggatcttgaaaggtga

SEQ ID NO: 16 - protein, 628 aa, synthetic construct
  REGION 1..628: note = translated from synthesized coding region
MDNNPNINECIPYNCLSNPEEVLLDGERISTGNSSIDISLSLVQFLVSNFVPGGGFAVGLIDFVWGIVGPSQWDAFLVQIEQLINERIAEFARNAAIANLEGLGNNFNIYVEAFKEWEEDPKNPATRTRVIDRFRILDGLLERDIPSFRISGFEVPLLSVYAQAANLHLAILRDSVIFGERWGLTTINVNENYNRLIRHIDEYADHCANTYNRGLNNLPKSTYQDWITYNRLRRDLTLTVLDIAAFFPNYDNRRYPIQPVGQLTREVYTDPLINFNPQLQSVAQLPTFNVMENSAIRNPHLFDILNNLTIFTDWFSVGRNFYWGGHRVISSLIGGGNITSPIYGREANQEPPRSFTFNGPVFRTLSNPTLRLLQQPWPAPPFNLRGVEGVEFSTPTNSFTYRGRGTVDSLTELPPEDNSVPPREGYSHRLCHATFVQRSGTPFLTTGVVFSWTHRSATLTNTIDPERINQIPLVKGFRVWGGTSVITGPGFTGGDILRRNTFGDFVSLQVNINSPITQRYRLRFRYASSRDARVIVLTGAASTGVGGQVSVNMPLQKTMEIGENLTSRTFRYTDFSNPFSFRANPDIIGISEQPLFGAGSISSGELYIDKIEIILADATLEAESDLER

SEQ ID NO: 17 - DNA, 1887 bp, synthetic construct
  misc_feature 1..1887: note = synthesized coding region
  source 1..1887: mol_type = other DNA
atggataacaaccccaacattaacgagtgcatcccgtacaactgcctctcgaatccagaagaagtgctcttggatggcgagaggatttcgactggcaacagctccatcgacatttccctctccttggttcagttccttgtgtctaacttcgtccctggcggtggcttccttgttggccttatcgacttcgtctggggaattttcggaccctcccagtgggatgcgtttctggtgcagatagagcagctgatcaacgagaggatcgctgagttcgcgagaaatgctgcaatcgccaaccttgaagggcttggcaacaacttcaacatctacgtggaggcgttcaaggagtgggaagaggaccctaagaatccagcgaccagaacgagggttatagatcggttccgcatcctcgatggccttttggagagggacatcccgagcttccgcatttcgggatttgaggttcctctgctctcagtctacgctcaagctgctaatctgcatctggccatcttgagggattcagtcatctttggcgaacgctggggtcttacgactatcaacgtgaacgagaactacaatcggttgattcggcacatagacgagtatgccgaccactgtgctaacacctacaataggggtctgaacaatctgccaaagtcaacgtatcaagactggataacctacaataggctcagacgggacctcactctcaccgtgctggacatagctgccttctttccgaactacgacaaccggagatatcctattcaacccgttggtcagctcactcgcgaggtctacaccgatcccctcatcaacttcaatccccagctgcaatcggtcgcacagctgcccaccttcaatgtgatggaaaactcagcgatccggaatccccatctgtttgacatacttaacaacctcactatcttcaccgattggttttcagttggacgcaacttctactggggagggcacagagtgatttcaagcctcattggaggagggaacattacatcgcctatctatggaagggaggccaaccaagagccaccaaggtctttcaccttcaacggtccggtgttcagaacacttagcaatcccacattgcgcttgctgcaacagccgtggccagcaccaccattcaatctgaggggagtggagggtgtggagttctcgacgcctacaaactcctttacgtacagaggcagagggacagtggactcactgacagaactcccacctgaggacaactctgttcctccgagggagggctactcgcaccggctttgccatgccaccttcgtccagaggtctggcacgccttttctgaccactggggttgtctttagctggactcaccgctcagcgacgctgaccaacacaatcgacccagagaggatcaatcagatccctctggtgaagggctttcgcgtttggggtggcacaagcgtgatcaccggacctggtttcactggtggggatatcctcagacgcaatacgtttggcgatttcgtgagccttcaagtcaacatcaattccccaatcacccagagatatcggctccgcttcagatacgcctcatccagagacgcaagggtcatcgtccttactggagcagccagcaccggagtcggaggccaagttagcgtcaacatgccgttgcagaaaacgatggaaatcggtgaaaacctcaccagcagaacctttcgctatacagatttcagcaaccctttctccttcagagccaatccggacataatcggcatatccgagcagcccttgttcggtgctgggtccatctcttctggcgagctgtacatcgacaagattgagatcattctcgcagatgcgactctggaggctgaatcggatcttgaaaggtga

SEQ ID NO: 18 - protein, 628 aa, synthetic construct
  REGION 1..628: note = translated from synthesized coding region
MDNNPNINECIPYNCLSNPEEVLLDGERISTGNSSIDISLSLVQFLVSNFVPGGGFLVGLIDFVWGIFGPSQWDAFLVQIEQLINERIAEFARNAAIANLEGLGNNFNIYVEAFKEWEEDPKNPATRTRVIDRFRILDGLLERDIPSFRISGFEVPLLSVYAQAANLHLAILRDSVIFGERWGLTTINVNENYNRLIRHIDEYADHCANTYNRGLNNLPKSTYQDWITYNRLRRDLTLTVLDIAAFFPNYDNRRYPIQPVGQLTREVYTDPLINFNPQLQSVAQLPTFNVMENSAIRNPHLFDILNNLTIFTDWFSVGRNFYWGGHRVISSLIGGGNITSPIYGREANQEPPRSFTFNGPVFRTLSNPTLRLLQQPWPAPPFNLRGVEGVEFSTPTNSFTYRGRGTVDSLTELPPEDNSVPPREGYSHRLCHATFVQRSGTPFLTTGVVFSWTHRSATLTNTIDPERINQIPLVKGFRVWGGTSVITGPGFTGGDILRRNTFGDFVSLQVNINSPITQRYRLRFRYASSRDARVIVLTGAASTGVGGQVSVNMPLQKTMEIGENLTSRTFRYTDFSNPFSFRANPDIIGISEQPLFGAGSISSGELYIDKIEIILADATLEAESDLER

SEQ ID NO: 19 - DNA, 1887 bp, synthetic construct
  misc_feature 1..1887: note = synthesized coding region
  source 1..1887: mol_type = other DNA
atggataacaaccccaacattaacgagtgcatcccgtacaactgcctctcgaatccagaagaagtgctcttggatggcgagaggatttcgactggcaacagctccatcgacatttccctctccttggttcagttccttgtgtctaacttcgtccctggcggtggcttccttgttggccttatcgacttcgtctggggaattatcggaccctcccagtgggatgcgtttctggtgcagatagagcagctgatcaacgagaggatcgctgagttcgcgagaaatgctgcaatcgccaaccttgaagggcttggcaacaacttcaacatctacgtggaggcgttcaaggagtgggaagaggaccctaagaatccagcgaccagaacgagggttatagatcggttccgcatcctcgatggccttttggagagggacatcccgagcttccgcatttcgggatttgaggttcctctgctctcagtctacgctcaagctgctaatctgcatctggccatcttgagggattcagtcatctttggcgaacgctggggtcttacgactatcaacgtgaacgagaactacaatcggttgattcggcacatagacgagtatgccgaccactgtgctaacacctacaataggggtctgaacaatctgccaaagtcaacgtatcaagactggataacctacaataggctcagacgggacctcactctcaccgtgctggacatagctgccttctttccgaactacgacaaccggagatatcctattcaacccgttggtcagctcactcgcgaggtctacaccgatcccctcatcaacttcaatccccagctgcaatcggtcgcacagctgcccaccttcaatgtgatggaaaactcagcgatccggaatccccatctgtttgacatacttaacaacctcactatcttcaccgattggttttcagttggacgcaacttctactggggagggcacagagtgatttcaagcctcattggaggagggaacattacatcgcctatctatggaagggaggccaaccaagagccaccaaggtctttcaccttcaacggtccggtgttcagaacacttagcaatcccacattgcgcttgctgcaacagccgtggccagcaccaccattcaatctgaggggagtggagggtgtggagttctcgacgcctacaaactcctttacgtacagaggcagagggacagtggactcactgacagaactcccacctgaggacaactctgttcctccgagggagggctactcgcaccggctttgccatgccaccttcgtccagaggtctggcacgccttttctgaccactggggttgtctttagctggactcaccgctcagcgacgctgaccaacacaatcgacccagagaggatcaatcagatccctctggtgaagggctttcgcgtttggggtggcacaagcgtgatcaccggacctggtttcactggtggggatatcctcagacgcaatacgtttggcgatttcgtgagccttcaagtcaacatcaattccccaatcacccagagatatcggctccgcttcagatacgcctcatccagagacgcaagggtcatcgtccttactggagcagccagcaccggagtcggaggccaagttagcgtcaacatgccgttgcagaaaacgatggaaatcggtgaaaacctcaccagcagaacctttcgctatacagatttcagcaaccctttctccttcagagccaatccggacataatcggcatatccgagcagcccttgttcggtgctgggtccatctcttctggcgagctgtacatcgacaagattgagatcattctcgcagatgcgactctggaggctgaatcggatcttgaaaggtga

SEQ ID NO: 20 - protein, 628 aa, synthetic construct
  REGION 1..628: note = translated from synthesized coding region
MDNNPNINECIPYNCLSNPEEVLLDGERISTGNSSIDISLSLVQFLVSNFVPGGGFLVGLIDFVWGIIGPSQWDAFLVQIEQLINERIAEFARNAAIANLEGLGNNFNIYVEAFKEWEEDPKNPATRTRVIDRFRILDGLLERDIPSFRISGFEVPLLSVYAQAANLHLAILRDSVIFGERWGLTTINVNENYNRLIRHIDEYADHCANTYNRGLNNLPKSTYQDWITYNRLRRDLTLTVLDIAAFFPNYDNRRYPIQPVGQLTREVYTDPLINFNPQLQSVAQLPTFNVMENSAIRNPHLFDILNNLTIFTDWFSVGRNFYWGGHRVISSLIGGGNITSPIYGREANQEPPRSFTFNGPVFRTLSNPTLRLLQQPWPAPPFNLRGVEGVEFSTPTNSFTYRGRGTVDSLTELPPEDNSVPPREGYSHRLCHATFVQRSGTPFLTTGVVFSWTHRSATLTNTIDPERINQIPLVKGFRVWGGTSVITGPGFTGGDILRRNTFGDFVSLQVNINSPITQRYRLRFRYASSRDARVIVLTGAASTGVGGQVSVNMPLQKTMEIGENLTSRTFRYTDFSNPFSFRANPDIIGISEQPLFGAGSISSGELYIDKIEIILADATLEAESDLER

SEQ ID NO: 21 - DNA, 1887 bp, synthetic construct
  misc_feature 1..1887: note = synthesized coding region
  source 1..1887: mol_type = other DNA
atggataacaaccccaacattaacgagtgcatcccgtacaactgcctctcgaatccagaagaagtgctcttggatggcgagaggatttcgactggcaacagctccatcgacatttccctctccttggttcagttccttgtgtctaacttcgtccctggcggtggcttccttgttggccttatcgacttcgtctggggaattgtcggaccctcccaggcggatgcgtttctggtgcagatagagcagctgatcaacgagaggatcgctgagttcgcgagaaatgctgcaatcgccaaccttgaagggcttggcaacaacttcaacatctacgtggaggcgttcaaggagtgggaagaggaccctaagaatccagcgaccagaacgagggttatagatcggttccgcatcctcgatggccttttggagagggacatcccgagcttccgcatttcgggatttgaggttcctctgctctcagtctacgctcaagctgctaatctgcatctggccatcttgagggattcagtcatctttggcgaacgctggggtcttacgactatcaacgtgaacgagaactacaatcggttgattcggcacatagacgagtatgccgaccactgtgctaacacctacaataggggtctgaacaatctgccaaagtcaacgtatcaagactggataacctacaataggctcagacgggacctcactctcaccgtgctggacatagctgccttctttccgaactacgacaaccggagatatcctattcaacccgttggtcagctcactcgcgaggtctacaccgatcccctcatcaacttcaatccccagctgcaatcggtcgcacagctgcccaccttcaatgtgatggaaaactcagcgatccggaatccccatctgtttgacatacttaacaacctcactatcttcaccgattggttttcagttggacgcaacttctactggggagggcacagagtgatttcaagcctcattggaggagggaacattacatcgcctatctatggaagggaggccaaccaagagccaccaaggtctttcaccttcaacggtccggtgttcagaacacttagcaatcccacattgcgcttgctgcaacagccgtggccagcaccaccattcaatctgaggggagtggagggtgtggagttctcgacgcctacaaactcctttacgtacagaggcagagggacagtggactcactgacagaactcccacctgaggacaactctgttcctccgagggagggctactcgcaccggctttgccatgccaccttcgtccagaggtctggcacgccttttctgaccactggggttgtctttagctggactcaccgctcagcgacgctgaccaacacaatcgacccagagaggatcaatcagatccctctggtgaagggctttcgcgtttggggtggcacaagcgtgatcaccggacctggtttcactggtggggatatcctcagacgcaatacgtttggcgatttcgtgagccttcaagtcaacatcaattccccaatcacccagagatatcggctccgcttcagatacgcctcatccagagacgcaagggtcatcgtccttactggagcagccagcaccggagtcggaggccaagttagcgtcaacatgccgttgcagaaaacgatggaaatcggtgaaaacctcaccagcagaacctttcgctatacagatttcagcaaccctttctccttcagagccaatccggacataatcggcatatccgagcagcccttgttcggtgctgggtccatctcttctggcgagctgtacatcgacaagattgagatcattctcgcagatgcgactctggaggctgaatcggatcttgaaaggtga

SEQ ID NO: 22 - protein, 628 aa, synthetic construct
  REGION 1..628: note = translated from synthesized coding region
MDNNPNINECIPYNCLSNPEEVLLDGERISTGNSSIDISLSLVQFLVSNFVPGGGFLVGLIDFVWGIVGPSQADAFLVQIEQLINERIAEFARNAAIANLEGLGNNFNIYVEAFKEWEEDPKNPATRTRVIDRFRILDGLLERDIPSFRISGFEVPLLSVYAQAANLHLAILRDSVIFGERWGLTTINVNENYNRLIRHIDEYADHCANTYNRGLNNLPKSTYQDWITYNRLRRDLTLTVLDIAAFFPNYDNRRYPIQPVGQLTREVYTDPLINFNPQLQSVAQLPTFNVMENSAIRNPHLFDILNNLTIFTDWFSVGRNFYWGGHRVISSLIGGGNITSPIYGREANQEPPRSFTFNGPVFRTLSNPTLRLLQQPWPAPPFNLRGVEGVEFSTPTNSFTYRGRGTVDSLTELPPEDNSVPPREGYSHRLCHATFVQRSGTPFLTTGVVFSWTHRSATLTNTIDPERINQIPLVKGFRVWGGTSVITGPGFTGGDILRRNTFGDFVSLQVNINSPITQRYRLRFRYASSRDARVIVLTGAASTGVGGQVSVNMPLQKTMEIGENLTSRTFRYTDFSNPFSFRANPDIIGISEQPLFGAGSISSGELYIDKIEIILADATLEAESDLER

SEQ ID NO: 23 - DNA, 1887 bp, synthetic construct
  misc_feature 1..1887: note = synthesized coding region
  source 1..1887: mol_type = other DNA
atggataacaaccccaacattaacgagtgcatcccgtacaactgcctctcgaatccagaagaagtgctcttggatggcgagaggatttcgactggcaacagctccatcgacatttccctctccttggttcagttccttgtgtctaacttcgtccctggcggtggcttccttgttggccttatcgacttcgtctggggaattgtcggaccctcccagtgggatgcgtttctggtgcagatagagcagctgatcaacgagaggatcgctgagttcgcgagaaatgctgcaatcgccaaccttgaagggcttggcaacaacttcaacatctacgtggaggcgttcaaggagtgggaagaggaccctaagaatccagcgaccagaacgagggttatagatcggttccgcatcctcgatggccttttggagagggacatcccgagcttccgcatttcgggatttgaggttcctctgctctcagtctacgctcaagctgctaatctgcatctggccatcttgagggattcagtcatctttggcgaacgctggggtcttacgactatcaacgtgaacgagaactacaatcggttgattcggcacatagacgagtatgccgaccactgtgctaacacctacaataggggtctgaacaatctgccaaagtcaacgtatcaagactggataacctacaataggctcagacgggacctcactctcaccgtgctggacatagctgccttctttccgaactacgacaaccggagatatcctattcaacccgttggtcagctcactcgcgaggtctacaccgatcccctcatcaacttcaatccccagctgcaatcggtcgcacagctgcccaccttcaatgtgatggaaaactcagcgatccggaatccccatctgtttgacatacttaacaacctcactatcttcaccgattggttttcagttggacgcaacttctactggggagggcacagagtgatttcaagcctcattggaggagggaacattacatcgcctatctatggaagggaggccaaccaagagccaccaaggtctttcaccttcaacggtccggtgttcagaacacttagcaatcccacattgcgcttgctgcaacagccgtggccagcaccaccattcaatctgaggggagtggagggtgtggagttctcgacgcctacaaactcctttacgtacagaggcagagggacagtggactcactgacagaactcccacctgaggacaactctgttcctccgagggagggctactcgcaccggctttgccatgccaccttcgtccagaggtctggcacgccttttctgaccactggggttgtctttagctggactcaccgctcagcgacgctgaccaacacaatcgacccagagaggatcaatcagatccctctggtgaagggctttcgcgtttggggtggcacaagcgtgatcaccggacctggtttcactggtggggatatcctcagacgcaatacgtttggcgatttcgtgagccttcaagtcaacatcaattccccaatcacccagagatatcggctccgcttcagatacgcctcatccagagacgcaagggtcatcgtccttactggagcagccagcaccggagtcggaggccaagttagcgtcaacatgccgttgcagaaaacgatggaaatcggtgaaaacctcaccagcagaacctttcgctatacagatttcagcaaccctttctccttcagagccaatccggacataatcggcatatccgagcagcccttgatgggtgctgggtccatctcttctggcgagctgtacatcgacaagattgagatcattctcgcagatgcgactctggaggctgaatcggatcttgaaaggtga

SEQ ID NO: 24 - protein, 628 aa, synthetic construct
  REGION 1..628: note = translated from synthesized coding region
MDNNPNINECIPYNCLSNPEEVLLDGERISTGNSSIDISLSLVQFLVSNFVPGGGFLVGLIDFVWGIVGPSQWDAFLVQIEQLINERIAEFARNAAIANLEGLGNNFNIYVEAFKEWEEDPKNPATRTRVIDRFRILDGLLERDIPSFRISGFEVPLLSVYAQAANLHLAILRDSVIFGERWGLTTINVNENYNRLIRHIDEYADHCANTYNRGLNNLPKSTYQDWITYNRLRRDLTLTVLDIAAFFPNYDNRRYPIQPVGQLTREVYTDPLINFNPQLQSVAQLPTFNVMENSAIRNPHLFDILNNLTIFTDWFSVGRNFYWGGHRVISSLIGGGNITSPIYGREANQEPPRSFTFNGPVFRTLSNPTLRLLQQPWPAPPFNLRGVEGVEFSTPTNSFTYRGRGTVDSLTELPPEDNSVPPREGYSHRLCHATFVQRSGTPFLTTGVVFSWTHRSATLTNTIDPERINQIPLVKGFRVWGGTSVITGPGFTGGDILRRNTFGDFVSLQVNINSPITQRYRLRFRYASSRDARVIVLTGAASTGVGGQVSVNMPLQKTMEIGENLTSRTFRYTDFSNPFSFRANPDIIGISEQPLMGAGSISSGELYIDKIEIILADATLEAESDLER

SEQ ID NO: 25 - DNA, 1887 bp, synthetic construct
  misc_feature 1..1887: note = synthesized coding region
  source 1..1887: mol_type = other DNA
atggataacaaccccaacattaacgagtgcatcccgtacaactgcctctcgaatccagaagaagtgctcttggatggcgagaggatttcgactggcaacagctccatcgacatttccctctccttggttcagttccttgtgtctaacttcgtccctggcggtggcttccttgttggccttatcgacttcgtctggggaattgtcggaccctcccagtgggatgcgtttctggtgcagatagagcagctgatcaacgagaggatcgctgagttcgcgagaaatgctgcaatcgccaaccttgaagggcttggcaacaacttcaacatctacgtggaggcgttcaaggagtgggaagaggaccctaagaatccagcgaccagaacgagggttatagatcggttccgcatcctcgatggccttttggagagggacatcccgagcttccgcatttcgggatttgaggttcctctgctctcagtctacgctcaagctgctaatctgcatctggccatcttgagggattcagtcatctttggcgaacgctggggtcttacgactatcaacgtgaacgagaactacaatcggttgattcggcacatagacgagtatgccgaccactgtgctaacacctacaataggggtctgaacaatctgccaaagtcaacgtatcaagactggataacctacaataggctcagacgggacctcactctcaccgtgctggacatagctgccttctttccgaactacgacaaccggagatatcctattcaacccgttggtcagctcactcgcgaggtctacaccgatcccctcatcaacttcaatccccagctgcaatcggtcgcacagctgcccaccttcaatgtgatggaaaactcagcgatccggaatccccatctgtttgacatacttaacaacctcactatcttcaccgattggttttcagttggacgcaacttctactggggagggcacagagtgatttcaagcctcattggaggagggaacattacatcgcctatctatggaagggaggccaaccaagagccaccaaggtctttcaccttcaacggtccggtgttcagaacacttagcaatcccacattgcgcttgctgcaacagccgtggccagcaccaccattcaatctgaggggagtggagggtgtggagttctcgacgcctacaaactcctttacgtacagaggcagagggacagtggactcactgacagaactcccacctgaggacaactctgttcctccgagggagggctactcgcaccggctttgccatgccaccttcgtccagaggtctggcacgccttttctgaccactggggttgtctttagctggactcaccgctcagcgacgctgaccaacacaatcgacccagagaggatcaatcagatccctctggtgaagggctttcgcgtttggggtggcacaagcgtgatcaccggacctggtttcactggtggggatatcctcagacgcaatacgtttggcgatttcgtgagccttcaagtcaacatcaattccccaatcacccagagatatcggctccgcttcagatacgcctcatccagagacgcaagggtcatcgtccttactggagcagccagcaccggagtcggaggccaagttagcgtcaacatgccgttgcagaaaacgatggaaatcggtgaaaacctcaccagcagaacctttcgctatacagatttcagcaaccctttctccttcagagccaatccggacataatcggcatatccgagcagcccttggccggtgctgggtccatctcttctggcgagctgtacatcgacaagattgagatcattctcgcagatgcgactctggaggctgaatcggatcttgaaaggtga

SEQ ID NO: 26 - protein, 628 aa, synthetic construct
  REGION 1..628: note = translated from synthesized coding region
MDNNPNINECIPYNCLSNPEEVLLDGERISTGNSSIDISLSLVQFLVSNFVPGGGFLVGLIDFVWGIVGPSQWDAFLVQIEQLINERIAEFARNAAIANLEGLGNNFNIYVEAFKEWEEDPKNPATRTRVIDRFRILDGLLERDIPSFRISGFEVPLLSVYAQAANLHLAILRDSVIFGERWGLTTINVNENYNRLIRHIDEYADHCANTYNRGLNNLPKSTYQDWITYNRLRRDLTLTVLDIAAFFPNYDNRRYPIQPVGQLTREVYTDPLINFNPQLQSVAQLPTFNVMENSAIRNPHLFDILNNLTIFTDWFSVGRNFYWGGHRVISSLIGGGNITSPIYGREANQEPPRSFTFNGPVFRTLSNPTLRLLQQPWPAPPFNLRGVEGVEFSTPTNSFTYRGRGTVDSLTELPPEDNSVPPREGYSHRLCHATFVQRSGTPFLTTGVVFSWTHRSATLTNTIDPERINQIPLVKGFRVWGGTSVITGPGFTGGDILRRNTFGDFVSLQVNINSPITQRYRLRFRYASSRDARVIVLTGAASTGVGGQVSVNMPLQKTMEIGENLTSRTFRYTDFSNPFSFRANPDIIGISEQPLAGAGSISSGELYIDKIEIILADATLEAESDLER

SEQ ID NO: 27 - DNA, 1887 bp, synthetic construct
  misc_feature 1..1887: note = synthesized coding region
  source 1..1887: mol_type = other DNA
atggataacaaccccaacattaacgagtgcatcccgtacaactgcctctcgaatccagaagaagtgctcttggatggcgagaggatttcgactggcaacagctccatcgacatttccctctccttggttcagttccttgtgtctaacttcgtccctggcgccggcttccttgttggccttatcgacttcgtctggggaattgtcggaccctcccagatggatgcgtttctggtgcagatagagcagctgatcaacgagaggatcgctgagttcgcgagaaatgctgcaatcgccaaccttgaagggcttggcaacaacttcaacatctacgtggaggcgttcaaggagtgggaagaggaccctaagaatccagcgaccagaacgagggttatagatcggttccgcatcctcgatggccttttggagagggacatcccgagcttccgcatttcgggatttgaggttcctctgctctcagtctacgctcaagctgctaatctgcatctggccatcttgagggattcagtcatctttggcgaacgctggggtcttacgactatcaacgtgaacgagaactacaatcggttgattcggcacatagacgagtatgccgaccactgtgctaacacctacaataggggtctgaacaatctgccaaagtcaacgtatcaagactggataacctacaataggctcagacgggacctcactctcaccgtgctggacatagctgccttctttccgaactacgacaaccggagatatcctattcaacccgttggtcagctcactcgcgaggtctacaccgatcccctcatcaacttcaatccccagctgcaatcggtcgcacagctgcccaccttcaatgtgatggaaaactcagcgatccggaatccccatctgtttgacatacttaacaacctcactatcttcaccgattggttttcagttggacgcaacttctactggggagggcacagagtgatttcaagcctcattggaggagggaacattacatcgcctatctatggaagggaggccaaccaagagccaccaaggtctttcaccttcaacggtccggtgttcagaacacttagcaatcccacattgcgcttgctgcaacagccgtggccagcaccaccattcaatctgaggggagtggagggtgtggagttctcgacgcctacaaactcctttacgtacagaggcagagggacagtggactcactgacagaactcccacctgaggacaactctgttcctccgagggagggctactcgcaccggctttgccatgccaccttcgtccagaggtctggcacgccttttctgaccactggggttgtctttagctggactcaccgctcagcgacgctgaccaacacaatcgacccagagaggatcaatcagatccctctggtgaagggctttcgcgtttggggtggcacaagcgtgatcaccggacctggtttcactggtggggatatcctcagacgcaatacgtttggcgatttcgtgagccttcaagtcaacatcaattccccaatcacccagagatatcggctccgcttcagatacgcctcatccagagacgcaagggtcatcgtccttactggagcagccagcaccggagtcggaggccaagttagcgtcaacatgccgttgcagaaaacgatggaaatcggtgaaaacctcaccagcagaacctttcgctatacagatttcagcaaccctttctccttcagagccaatccggacataatcggcatatccgagcagcccttgttcggtgctgggtccatctcttctggcgagctgtacatcgacaagattgagatcattctcgcagatgcgactctggaggctgaatcggatcttgaaaggtga

SEQ ID NO: 28 - protein, 628 aa, synthetic construct
  REGION 1..628: note = translated from synthesized coding region
MDNNPNINECIPYNCLSNPEEVLLDGERISTGNSSIDISLSLVQFLVSNFVPGAGFLVGLIDFVWGIVGPSQMDAFLVQIEQLINERIAEFARNAAIANLEGLGNNFNIYVEAFKEWEEDPKNPATRTRVIDRFRILDGLLERDIPSFRISGFEVPLLSVYAQAANLHLAILRDSVIFGERWGLTTINVNENYNRLIRHIDEYADHCANTYNRGLNNLPKSTYQDWITYNRLRRDLTLTVLDIAAFFPNYDNRRYPIQPVGQLTREVYTDPLINFNPQLQSVAQLPTFNVMENSAIRNPHLFDILNNLTIFTDWFSVGRNFYWGGHRVISSLIGGGNITSPIYGREANQEPPRSFTFNGPVFRTLSNPTLRLLQQPWPAPPFNLRGVEGVEFSTPTNSFTYRGRGTVDSLTELPPEDNSVPPREGYSHRLCHATFVQRSGTPFLTTGVVFSWTHRSATLTNTIDPERINQIPLVKGFRVWGGTSVITGPGFTGGDILRRNTFGDFVSLQVNINSPITQRYRLRFRYASSRDARVIVLTGAASTGVGGQVSVNMPLQKTMEIGENLTSRTFRYTDFSNPFSFRANPDIIGISEQPLFGAGSISSGELYIDKIEIILADATLEAESDLER

SEQ ID NO: 29 - DNA, 1887 bp, synthetic construct
  misc_feature 1..1887: note = synthesized coding region
  source 1..1887: mol_type = other DNA
atggataacaaccccaacattaacgagtgcatcccctacaactgcctctcgaatccagaagaagtgctcttggatggcgagaggatttcgactggcaacagctccatagacatttccctctccttggttcagttccttgtgtctaacttcgtccctggcggtggcttccttgttggattaatcgacttcgtctggggaattgtcggaccctcccagtgggatgcgtttctggtgcagatagagcaattaattaatgaaaggatcgctgagttcgcgagaaatgctgcaatcgccaaccttgaagggcttggcaacaacttcaacatctacgtggaagcatttaaggagtgggaagaggaccctaagaatccagcgaccagaacgagggttatagatcggttccgcatcctcgatggccttttggagagggacatcccgagcttccgcatttcgggatttgaggttcctctgctctcagtctacgctcaagctgctaatctgcatctggccatcttgagggattcagtcatctttggcgaacgctggggtcttacgactatcaacgtgaatgaaaactacaatcggttgattcggcacatagacgagtatgccgaccactgtgctaacacctacaataggggattaaataatctgccaaagtcaacgtatcaagactggataacatataataggctcagacgggacctcactctcaccgtgctggacatagctgccttctttccgaactacgacaaccggagatatcctattcaacccgttggtcagctcactcgcgaggtctacaccgatccattaattaatttcaatccccagctgcaatcggtcgcacagctgcccaccttcaatgtgatggaaaactcagcgatccggaatccccatctctttgacatacttaataatctcactatcttcaccgattggttttcagttggacgcaacttctactggggagggcacagagtgatttcaagcctcattggaggagggaacattacatcgcctatctatggaagggaggccaaccaggagccaccaaggtctttcaccttcaacggtccggtgttcagaacacttagcaatcccacattgcgcttgctgcaacagccgtggccagcaccaccattcaatctgaggggagtggagggtgtggagttctcgacgcctacaaactcctttacgtacagaggcagagggacagtggactcactgacagaactcccacctgaggacaactctgttcctccgagggagggctactcgcaccggctttgccatgccaccttcgtccagaggtctggcacgccttttctgaccactggggttgtctttagctggactcaccgctcagcgacgctgaccaatacaatcgacccagagagaattaatcaaatccctctggtgaagggctttcgcgtttggggtggcacaagcgtgatcaccggacctggtttcactggtggggatatcctcagacgcaatacgtttggcgatttcgtgagccttcaagtcaacattaattccccaatcacccagagatatcggctccgcttcagatacgcctcatccagagacgcaagggtcatcgtccttactggagcagccagcaccggagtcggaggccaagttagcgtcaacatgccgttgcagaaaacgatggaaatcggtgaaaacctcaccagcagaacctttcgctatacagatttcagcaaccctttctccttcagagccaatccggatataatcggcatatccgagcagcccttgttcggtgctgggtccatctcttcaggcgagctgtacatcgataaaattgagatcattctcgcagatgcgactctcgaggctgaatcggatcttgaaaggtga

SEQ ID NO: 30 - protein, 628 aa, Bacillus thuringiensis; identical to SEQ ID NO: 10

SEQ ID NO: 31 - DNA, 1887 bp, synthetic construct
  misc_feature 1..1887: note = synthesized coding region
  source 1..1887: mol_type = other DNA
atggataacaacccgaacatcaacgagtgcatcccctacaactgcctgagcaaccccgaggaggtgctgctggacggcgagaggatctcaaccggcaacagcagcatcgacatcagcctgtccctggtgcagttcctggtgagcaacttcgtgccgggcggcggcttcctggtgggattaatcgacttcgtgtggggcatcgtcggcccgtcccagtgggacgccttcctggttcagatcgagcaattaattaatgaaaggatagcagagttcgcgaggaacgcggccatcgccaacctggagggcctgggcaacaacttcaacatctacgtggaagcatttaaggagtgggaggaggaccccaagaacccggccacgaggacgagggtgatcgaccgctttcgcatcctggacggcctgctggagagggacatcccgtccttcagaatcagcggcttcgaggtcccgctgctgtccgtgtacgcgcaagcggccaacctgcacctggcgatcctgagggactccgtgatattcggcgagaggtggggcctgaccaccatcaacgtgaatgaaaactacaaccggctgataaggcacatcgacgagtacgccgaccactgcgccaacacctacaataggggattaaataatctgcccaagagcacctaccaagactggatcacatataaccggctgaggagggacctgaccctgaccgtgctggacatcgccgcgttcttcccgaactacgacaataggcgctacccgatccagccggtgggccagctgacccgcgaggtgtacaccgacccattaattaatttcaacccgcagctccagtccgtggcccagctgccgaccttcaacgtgatggagaacagcgccatccggaacccgcacctgttcgacatcctgaataatctgaccatcttcaccgactggttctcagtgggccggaacttctactggggcggccatagggtgatctccagcctgatcggcggcggcaacatcacctccccgatctacgggagggaggcgaaccaggagccgccgaggtccttcaccttcaacggcccggtgtttaggaccctgtccaacccgaccctgaggctgctccagcagccgtggccggcgccgccgttcaacctgaggggcgtggagggcgtggagttcagcaccccgaccaacagcttcacctaccgggggaggggcaccgtggactcactgaccgagctgccgccggaggacaacagcgtgccgccgagggagggctacagccataggctgtgccacgccaccttcgtgcagaggagcggcaccccgttcttgacgaccggcgtggtgttctcctggacccaccggagcgcgaccctgaccaatacaatcgacccggagagaattaatcaaatcccgctggtgaagggcttccgggtgtggggcggcacctccgtgatcaccgggccgggctttaccggcggcgacatcctgaggaggaacacgttcggcgacttcgtgagcctccaagtgaacattaatagcccgatcacccagcgctaccggctgaggttccgctacgcgtcaagccgcgacgcgagggtgatcgtgctgaccggcgcggcctcaaccggcgtgggcggccaagtgtccgtgaacatgccgctgcaaaagacgatggagatcggcgagaacctgacctcaaggaccttccgctacaccgacttcagcaacccgttcagctttagggccaacccggatataatcggcatcagcgagcagccgctgttcggcgccggctccatctcaagcggcgagctgtacatcgataaaatcgagatcatcctggcggacgcgaccttggaggccgagtccgacctggagaggtga

SEQ ID NO: 32 - protein, 628 aa, Bacillus thuringiensis; identical to SEQ ID NO: 10

SEQ ID NO: 33 - DNA, 1887 bp, synthetic construct
  misc_feature 1..1887: note = synthesized coding region
  source 1..1887: mol_type = other DNA
atggacaacaatcccaatatcaatgagtgcatcccatacaactgcctttcaaatcccgaggaagtcttactggatggggaaaggattagcactggcaattccagcattgacatttccttgagtctcgttcaattccttgtgagcaactttgtgcctggcggagggttcttggttggtctcatagactttgtttggggaattgtaggaccatcacagtgggatgccttcttagtccagattgaacagcttatcaatgagagaatagctgagttcgcaagaaatgctgcaatcgctaacttggagggactcggcaacaacttcaacatctacgtggaggctttcaaagaatgggaagaagatcctaagaatccagctactaggactagagtcatcgataggtttaggattcttgatgggctgttggagcgtgacattccttcctttaggattagtggcttcgaggttcctctcctttctgtctatgctcaagcagccaatctccatctcgccattctccgtgattcagtaatcttcggagaaagatggggtttgacaacgatcaatgtgaatgagaactacaacagattgatcagacacattgatgagtatgctgatcattgtgctaacacatacaacagagggcttaacaacctcccgaaatcaacatatcaagattggataacctacaatagattgaggagggatctcacactgactgtactcgacattgctgctttctttcccaactacgataaccgtagatatcccattcaacccgtcggacaactcactcgtgaagtgtacacagatccccttatcaacttcaacccacagttacaaagtgttgcacagctgccaaccttcaatgtgatggaaaactctgccattaggaatccacatctgtttgacatacttaacaacttgactatctttacggattggtttagtgtggggaggaacttctattggggaggtcacagagtgattagctctcttatcggaggtgggaacattacttcaccaatctatggacgtgaagcaaatcaagaaccaccacgttcattcaccttcaatggacccgttttcagaacactgtccaatccgactctgagactgttgcagcaaccttggccagcacctcctttcaacttgaggggtgtagagggagtcgagttttccacacctactaacagtttcacctataggggtcgtggaactgttgattctcttactgaacttcctccagaggacaattctgttcctccgagggaaggctattcacaccgtttgtgtcatgcaacttttgttcagaggtctggcacacctttcttaacaactggcgtcgttttcagctggacacataggagtgcaaccctcacgaatactatcgatccagagagaatcaatcagattcctcttgttaagggctttagggtgtggggtgggacctcagtgataactggtcccggtttcaccggaggagacatacttagaaggaacacctttggggatttcgtttctcttcaagtaaacatcaattctcctatcacccagagatacagacttaggtttagatatgctagtagccgtgacgccagagtcatagtgttgactggtgctgcatcaactggtgttggtgggcaagtttcagtgaacatgccacttcaaaagacaatggagataggtgagaacttgacgtccagaaccttcagatacactgacttttctaatcctttctcttttcgtgctaatccagacatcataggaatcagtgaacaaccgttgtttggagctggttccatttctagtggagaattgtacattgacaagattgagatcatacttgccgatgccacgctcgaggctgaatctgatctcgaaaggtga

SEQ ID NO: 34 - protein, 628 aa, Bacillus thuringiensis; identical to SEQ ID NO: 10

SEQ ID NO: 35 - DNA, 3495 bp, synthetic construct
  misc_feature 1..3495: note = synthesized coding region
  source 1..3495: mol_type = other DNA
atggacaacaatcccaatatcaatgagtgcatcccatacaactgcctttcaaatcccgaggaagtcttactggatggggaaaggattagcactggcaattccagcattgacatttccttgagtctcgttcaattccttgtgagcaactttgtgcctggcggagggttcttggttggtctcatagactttgtttggggaattgtaggaccatcacagtgggatgccttcttagtccagattgaacagcttatcaatgagagaatagctgagttcgcaagaaatgctgcaatcgctaacttggagggactcggcaacaacttcaacatctacgtggaggctttcaaagaatgggaagaagatcctaagaatccagctactaggactagagtcatcgataggtttaggattcttgatgggctgttggagcgtgacattccttcctttaggattagtggcttcgaggttcctctcctttctgtctatgctcaagcagccaatctccatctcgccattctccgtgattcagtaatcttcggagaaagatggggtttgacaacgatcaatgtgaatgagaactacaacagattgatcagacacattgatgagtatgctgatcattgtgctaacacatacaacagagggcttaacaacctcccgaaatcaacatatcaagattggataacctacaatagattgaggagggatctcacactgactgtactcgacattgctgctttctttcccaactacgataaccgtagatatcccattcaacccgtcggacaactcactcgtgaagtgtacacagatccccttatcaacttcaacccacagttacaaagtgttgcacagctgccaaccttcaatgtgatggaaaactctgccattaggaatccacatctgtttgacatacttaacaacttgactatctttacggattggtttagtgtggggaggaacttctattggggaggtcacagagtgattagctctcttatcggaggtgggaacattacttcaccaatctatggacgtgaagcaaatcaagaaccaccacgttcattcaccttcaatggacccgttttcagaacactgtccaatccgactctgagactgttgcagcaaccttggccagcacctcctttcaacttgaggggtgtagagggagtcgagttttccacacctactaacagtttcacctataggggtcgtggaactgttgattctcttactgaacttcctccagaggacaattctgttcctccgagggaaggctattcacaccgtttgtgtcatgcaacttttgttcagaggtctggcacacctttcttaacaactggcgtcgttttcagctggacacataggagtgcaaccctcacgaatactatcgatccagagagaatcaatcagattcctcttgttaagggctttagggtgtggggtgggacctcagtgataactggtcccggtttcaccggaggagacatacttagaaggaacacctttggggatttcgtttctcttcaagtaaacatcaattctcctatcacccagagatacagacttaggtttagatatgctagtagccgtgacgccagagtcatagtgttgactggtgctgcatcaactggtgttggtgggcaagtttcagtgaacatgccacttcaaaagacaatggagataggtgagaacttgacgtccagaaccttcagatacactgacttttctaatcctttctcttttcgtgctaatccagacatcataggaatcagtgaacaaccgttgtttggagctggttccatttctagtggagaattgtacattgacaagattgagatcatacttgccgatgccacgctcgaggctgaatctgatctcgaaagggcacagaaagctgtaaacgcattgtttacaagttctaatcaaatcggactcaaaaccgatgttacggactatcacatagatagggtttctaatcttgtggaatgtctttcagatgagttttgtttagatgagaagaaagaactttcagaaaaggtcaagcacgccaaaagactgtccgatgaaaggaatctccttcaagacccaaactttcgtggaatcaataggcagctcgacagaggttggagagggagcacagatatcaccattcaaggaggagatgacgttttcaaagagaactatgtcaccttgttaggcacctttgatgagtgctatccaacttatctgtatcagaagattgatgaatccaagctgaaggcttacacaagatatcagctcagaggatacatcgaggactcccaagatttggagatatacttgattcgttacaatgcaaaacatgagaccgtgaatgttcctggtactggaagtctctggccactgtctgctccgtcacctattgggaaatgtgcccatcactcccaccatttctcattggacatagacgttggctgcacagatttgaatgaagatttgggtgtttgggtcatcttcaagatcaaaactcaagacggacacgctcgtttaggaaacttagagtttcttgaagagaagcccttggttggggaggcacttgccagagtaaagagagctgaaaagaagtggagagataagagggagaaacttgagtgggagactaacattgtgtacaaggaagccaaagaaagcgtggatgctcttttcgtgaactctcagtatgataggttacaagcagacaccaacatagcaatgatacatgcagctgacaaaagagtccattctattcgtgaggcttacttgccagaacttagtgtgattcccggtgtcaacgctgccattttcgaggaattggaaggaagaatctttacggctttcagcctctatgacgctaggaatgttatcaagaatggtgatttcaacaatggcctctcatgttggaatgtgaaaggtcatgttgatgtagaggagcaaaacaatcaccgtagcgtgctggttgtcccagaatgggaagccgaagtaagccaagaagttagagtttgccctggaagaggctacattctgcgtgtcaccgcttacaaagaaggatatggcgaagggtgcgtgactattcatgagattgagaacaatactgacgaacttaagttttcaaactgcgtcgaggaggaagtgtatcctaacaacacagtgacttgtaatgactatacagcaacgcaagaggaatacgaggggacatacaccagtcgtaatcgtggttatgatggtgcttatgaaagcaattcatccgttccagctgactatgccagtgcctacgaagagaaggcttacacggatggcagaagagataacccatgtgagtccaacagaggttatggtgattacactcctcttccagctggttacgtgactaaagagttagagtactttccggagactgataaggtttggattgaaatcggagagacagaagggacattcatagtagattcagttgagcttcttctcatggaagaatga

SEQ ID NO: 36 - protein, 1164 aa, synthetic construct
  REGION 1..1164: note = translated from synthesized coding region
MDNNPNINECIPYNCLSNPEEVLLDGERISTGNSSIDISLSLVQFLVSNFVPGGGFLVGLIDFVWGIVGPSQWDAFLVQIEQLINERIAEFARNAAIANLEGLGNNFNIYVEAFKEWEEDPKNPATRTRVIDRFRILDGLLERDIPSFRISGFEVPLLSVYAQAANLHLAILRDSVIFGERWGLTTINVNENYNRLIRHIDEYADHCANTYNRGLNNLPKSTYQDWITYNRLRRDLTLTVLDIAAFFPNYDNRRYPIQPVGQLTREVYTDPLINFNPQLQSVAQLPTFNVMENSAIRNPHLFDILNNLTIFTDWFSVGRNFYWGGHRVISSLIGGGNITSPIYGREANQEPPRSFTFNGPVFRTLSNPTLRLLQQPWPAPPFNLRGVEGVEFSTPTNSFTYRGRGTVDSLTELPPEDNSVPPREGYSHRLCHATFVQRSGTPFLTTGVVFSWTHRSATLTNTIDPERINQIPLVKGFRVWGGTSVITGPGFTGGDILRRNTFGDFVSLQVNINSPITQRYRLRFRYASSRDARVIVLTGAASTGVGGQVSVNMPLQKTMEIGENLTSRTFRYTDFSNPFSFRANPDIIGISEQPLFGAGSISSGELYIDKIEIILADATLEAESDLERAQKAVNALFTSSNQIGLKTDVTDYHIDRVSNLVECLSDEFCLDEKKELSEKVKHAKRLSDERNLLQDPNFRGINRQLDRGWRGSTDITIQGGDDVFKENYVTLLGTFDECYPTYLYQKIDESKLKAYTRYQLRGYIEDSQDLEIYLIRYNAKHETVNVPGTGSLWPLSAPSPIGKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYDRLQADTNIAMIHAADKRVHSIREAYLPELSVIPGVNAAIFEELEGRIFTAFSLYDARNVIKNGDFNNGLSCWNVKGHVDVEEQNNHRSVLVVPEWEAEVSQEVRVCPGRGYILRVTAYKEGYGEGCVTIHEIENNTDELKFSNCVEEEVYPNNTVTCNDYTATQEEYEGTYTSRNRGYDGAYESNSSVPADYASAYEEKAYTDGRRDNPCESNRGYGDYTPLPAGYVTKELEYFPETDKVWIEIGETEGTFIVDSVELLLMEE

SEQ ID NO: 37 - DNA, 3495 bp, synthetic construct
  misc_feature 1..3495: note = synthesized coding region
  source 1..3495: mol_type = other DNA
atggataacaatccgaacatcaatgaatgcatcccgtacaactgcctgagcaacccggaagaagtgctgttggatggagaacggatatcaactggtaattcatcaattgatatttctctgtcacttgttcagtttctggtatctaacttcgtcccaggcggaggattcctggttggattaatagattttgtatggggaatagttggcccttctcaatgggatgcatttctagtacaaattgaacaattaattaatgaaagaatagctgaatttgctaggaatgctgctattgctaatttagaaggattaggaaacaatttcaatatatatgtggaagcatttaaagaatgggaagaagatcctaagaatccagcaaccaggaccagagtaattgatcgctttcgtatacttgatgggctacttgaaagggacattccttcgtttcgaatttctggatttgaagtaccccttttatccgtttatgctcaagcggccaatctgcatctagctatattaagagattctgtaatttttggagaaagatggggattgacaacgataaatgtcaatgaaaactataatagactaattaggcatattgatgaatatgctgatcactgtgcaaatacgtataatcggggattaaataatttaccgaaatctacgtatcaagattggataacatataatcgattgcggagagacttaacattgactgtattagatatcgccgctttctttccaaactatgacaataggagatatccaattcagccagttggtcaactaacaagggaagtttatacggacccattaattaattttaatccacagttacagtctgtagctcaattacctacttttaacgttatggagaacagcgcaattagaaatcctcatttatttgatatattgaataatcttacaatctttacggattggtttagtgttggacgcaatttttattggggaggacatcgagtaatatctagccttataggaggtggtaacataacatctcctatatatggaagagaggcgaaccaggagcctccaagatcctttacttttaatggaccggtatttaggactttatcaaatcctactttacgattattacagcaaccttggccagcgccaccatttaatttacgtggtgttgaaggagtagaattttctacacctacaaatagctttacgtatcgaggaagaggtacggttgattctttaactgaattgccgcctgaggataatagtgtgccacctcgcgaaggatatagtcatcgtttatgtcatgcaacttttgttcaaagatctggaacaccttttttaacaactggtgtagtattttcttggacgcatcgtagtgcaactcttacaaatacaattgatccagagagaattaatcaaatacctttagtgaaaggatttagagtttgggggggcacctctgtcattacaggaccaggatttacaggaggggatatccttcgaagaaatacctttggtgattttgtatctctacaagtcaatattaattcaccaattacccaaagataccgtttaagatttcgttacgcttccagtagggatgcacgagttatagtattaacaggagcggcatccacaggagtgggaggccaagttagtgtaaatatgcctcttcagaaaactatggaaataggggagaacttaacatctagaacatttagatataccgattttagtaatcctttttcatttagagctaatccagatataattgggataagtgaacaacctctatttggtgcaggttctattagtagcggtgaactttatatagataaaattgaaattattctagcagatgcaacatttgaagcagaatctgatttagaaagagcacaaaaggcggtgaatgccctgtttacttcttccaatcaaatcgggttaaaaaccgatgtgacggactatcatatcgatcgagtatccaatttagttgagtgtttatctgatgaattttgtctggatgaaaaaaaagaattgtccgagaaagtcaaacatgcgaagcgacttagtgatgagcggaatttacttcaagatccaaactttagagggatcaatagacaactagaccgtggctggagaggaagtacggatattaccatccaaggaggcgatgacgtatccaaagagaattacgttacgctattgggtacctttgatgagtgctacttaacgtatttatatcaaaaaatagatgagtcgaaattaaaagcctatacccgttaccaattaagagggtatatcgaagatagtcaagacttagaaatctatttaattcgctacaatgccaaacacgaaacagtaaatgtgccaggtacgggttccttatggccgctttcagccccaagtccaatcggaaaatgtgcccatcattcccatcatttctccttggacattgatgttggatgtacagacttaaatgaggacttaggtgtatgggtgatattcaagattaagacgcaagatggccatgcaagactaggaaatctagaatttctcgaagagaaaccattagtaggagaagcactagctcgtgtgaaaagagcggagaaaaaatggagagacaaacgtgaaaaattggaatgggaaacaaatattgtttataaagaggcaaaagaatctgtagatgctttatttgtaaactctcaatatgatagattacaagcggataccaacatcgcgatgattcatgcggcagataaacgcgttcatagcattcgagaagcttatctgcctgagctgtctgtgattccgggtgtcaatgcggctatttttgaagaattagaagggcgtattttcactgcattctccctatatgatgcgagaaatgtcattaaaaatggtgattttaataatggcttatcctgctggaacgtgaaagggcatgtagatgtagaagaacaaaacaaccaccgttcggtccttgttgttccggaatgggaagcagaagtgtcacaagaagttcgtgtctgtccgggtcgtggctatatccttcgtgtcacagcgtacaaggagggatatggagaaggttgcgtaaccattcatgagatcgagaacaatacagacgaactgaagtttagcaactgtgtagaagaggaagtatatccaaacaacacggtaacgtgtaatgattatactgcgactcaagaagaatatgagggtacgtacacttctcgtaatcgaggatatgacggagcctatgaaagcaattcttctgtaccagctgattatgcatcagcctatgaagaaaaagcatatacagatggacgaagagacaatccttgtgaatctaacagaggatatggggattacacaccactaccagctggctatgtgacaaaagaattagagtacttcccagaaaccgataaggtatggattgagatcggagaaacggaaggaacattcatcgtggacagcgtggaattacttcttatggaggaataa

SEQ ID NO: 38 - protein, 1164 aa, synthetic construct
  REGION 1..1164: note = translated from synthesized coding region
MDNNPNINECIPYNCLSNPEEVLLDGERISTGNSSIDISLSLVQFLVSNFVPGGGFLVGLIDFVWGIVGPSQWDAFLVQIEQLINERIAEFARNAAIANLEGLGNNFNIYVEAFKEWEEDPKNPATRTRVIDRFRILDGLLERDIPSFRISGFEVPLLSVYAQAANLHLAILRDSVIFGERWGLTTINVNENYNRLIRHIDEYADHCANTYNRGLNNLPKSTYQDWITYNRLRRDLTLTVLDIAAFFPNYDNRRYPIQPVGQLTREVYTDPLINFNPQLQSVAQLPTFNVMENSAIRNPHLFDILNNLTIFTDWFSVGRNFYWGGHRVISSLIGGGNITSPIYGREANQEPPRSFTFNGPVFRTLSNPTLRLLQQPWPAPPFNLRGVEGVEFSTPTNSFTYRGRGTVDSLTELPPEDNSVPPREGYSHRLCHATFVQRSGTPFLTTGVVFSWTHRSATLTNTIDPERINQIPLVKGFRVWGGTSVITGPGFTGGDILRRNTFGDFVSLQVNINSPITQRYRLRFRYASSRDARVIVLTGAASTGVGGQVSVNMPLQKTMEIGENLTSRTFRYTDFSNPFSFRANPDIIGISEQPLFGAGSISSGELYIDKIEIILADATFEAESDLERAQKAVNALFTSSNQIGLKTDVTDYHIDRVSNLVECLSDEFCLDEKKELSEKVKHAKRLSDERNLLQDPNFRGINRQLDRGWRGSTDITIQGGDDVSKENYVTLLGTFDECYLTYLYQKIDESKLKAYTRYQLRGYIEDSQDLEIYLIRYNAKHETVNVPGTGSLWPLSAPSPIGKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYDRLQADTNIAMIHAADKRVHSIREAYLPELSVIPGVNAAIFEELEGRIFTAFSLYDARNVIKNGDFNNGLSCWNVKGHVDVEEQNNHRSVLVVPEWEAEVSQEVRVCPGRGYILRVTAYKEGYGEGCVTIHEIENNTDELKFSNCVEEEVYPNNTVTCNDYTATQEEYEGTYTSRNRGYDGAYESNSSVPADYASAYEEKAYTDGRRDNPCESNRGYGDYTPLPAGYVTKELEYFPETDKVWIEIGETEGTFIVDSVELLLMEE

SEQ ID NO: 39 - DNA, 2109 bp, synthetic construct
  misc_feature 1..2109: note = synthesized coding region
  source 1..2109: mol_type = other DNA
atggcacagagcagtaggatctgccacggtgtgcagaacccgtgcgtgatcatctcgaacctgagcaagtccaaccagaacaagtcaccgttctccgtgtccctcaagacccaccagcacccgagagcctacccgatcagcagctcctggggactgaagaagagtggcatgaccctgatcggctccgagctgagaccgctgaaggtgatgtccagcgtgtcagcggataacaacccgaacatcaacgagtgcatcccctacaactgcctgagcaaccccgaggaggtgctgctggacggcgagaggatctcaaccggcaacagcagcatcgacatcagcctgtccctggtgcagttcctggtgagcaacttcgtgccgggcggcggcttcctggtgggattaatcgacttcgtgtggggcatcgtcggcccgtcccagtgggacgccttcctggttcagatcgagcaattaattaatgaaaggatagcagagttcgcgaggaacgcggccatcgccaacctggagggcctgggcaacaacttcaacatctacgtggaagcatttaaggagtgggaggaggaccccaagaacccggccacgaggacgagggtgatcgaccgctttcgcatcctggacggcctgctggagagggacatcccgtccttcagaatcagcggcttcgaggtcccgctgctgtccgtgtacgcgcaagcggccaacctgcacctggcgatcctgagggactccgtgatattcggcgagaggtggggcctgaccaccatcaacgtgaatgaaaactacaaccggctgataaggcacatcgacgagtacgccgaccactgcgccaacacctacaataggggattaaataatctgcccaagagcacctaccaagactggatcacatataaccggctgaggagggacctgaccctgaccgtgctggacatcgccgcgttcttcccgaactacgacaataggcgctacccgatccagccggtgggccagctgacccgcgaggtgtacaccgacccattaattaatttcaacccgcagctccagtccgtggcccagctgccgaccttcaacgtgatggagaacagcgccatccggaacccgcacctgttcgacatcctgaataatctgaccatcttcaccgactggttctcagtgggccggaacttctactggggcggccatagggtgatctccagcctgatcggcggcggcaacatcacctccccgatctacgggagggaggcgaaccaggagccgccgaggtccttcaccttcaacggcccggtgtttaggaccctgtccaacccgaccctgaggctgctccagcagccgtggccggcgccgccgttcaacctgaggggcgtggagggcgtggagttcagcaccccgaccaacagcttcacctaccgggggaggggcaccgtggactcactgaccgagctgccgccggaggacaacagcgtgccgccgagggagggctacagccataggctgtgccacgccaccttcgtgcagaggagcggcaccccgttcttgacgaccggcgtggtgttctcctggacccaccggagcgcgaccctgaccaatacaatcgacccggagagaattaatcaaatcccgctggtgaagggcttccgggtgtggggcggcacctccgtgatcaccgggccgggctttaccggcggcgacatcctgaggaggaacacgttcggcgacttcgtgagcctccaagtgaacattaatagcccgatcacccagcgctaccggctgaggttccgctacgcgtcaagccgcgacgcgagggtgatcgtgctgaccggcgcggcctcaaccggcgtgggcggccaagtgtccgtgaacatgccgctgcaaaagacgatggagatcggcgagaacctgacctcaaggaccttccgctacaccgacttcagcaacccgttcagctttagggccaacccggatataatcggcatcagcgagcagccgctgttcggcgccggctccatctcaagcggcgagctgtacatcgataaaatcgagatcatcctggcggacgcgaccttggaggccgagtccgacctggagaggtga

SEQ ID NO: 40 - protein, 702 aa, synthetic construct
  REGION 1..702: note = translated from synthesized coding region
MAQSSRICHGVQNPCVIISNLSKSNQNKSPFSVSLKTHQHPRAYPISSSWGLKKSGMTLIGSELRPLKVMSSVSADNNPNINECIPYNCLSNPEEVLLDGERISTGNSSIDISLSLVQFLVSNFVPGGGFLVGLIDFVWGIVGPSQWDAFLVQIEQLINERIAEFARNAAIANLEGLGNNFNIYVEAFKEWEEDPKNPATRTRVIDRFRILDGLLERDIPSFRISGFEVPLLSVYAQAANLHLAILRDSVIFGERWGLTTINVNENYNRLIRHIDEYADHCANTYNRGLNNLPKSTYQDWITYNRLRRDLTLTVLDIAAFFPNYDNRRYPIQPVGQLTREVYTDPLINFNPQLQSVAQLPTFNVMENSAIRNPHLFDILNNLTIFTDWFSVGRNFYWGGHRVISSLIGGGNITSPIYGREANQEPPRSFTFNGPVFRTLSNPTLRLLQQPWPAPPFNLRGVEGVEFSTPTNSFTYRGRGTVDSLTELPPEDNSVPPREGYSHRLCHATFVQRSGTPFLTTGVVFSWTHRSATLTNTIDPERINQIPLVKGFRVWGGTSVITGPGFTGGDILRRNTFGDFVSLQVNINSPITQRYRLRFRYASSRDARVIVLTGAASTGVGGQVSVNMPLQKTMEIGENLTSRTFRYTDFSNPFSFRANPDIIGISEQPLFGAGSISSGELYIDKIEIILADATLEAESDLER

(19) United States
(12) Patent Application Publication
(10) Pub. No.: US 2026/0078153 A1
(43) Pub. Date: Mar. 19, 2026

(54) MODIFIED Cry1Ca TOXINS USEFUL FOR CONTROL OF INSECT PESTS

(71) Applicant: CORTEVA AGRISCIENCE LLC, INDIANAPOLIS, IN (US)

(72) Inventors: JOEL J. SHEETS, SAN LUIS OBISPO, CA (US); JANNA MAI ARMSTRONG, SEATTLE, WA (US); TRISTAN E. CORAM, ZIONSVILLE, IN (US); AUDREY JANE ETTER, INDIANAPOLIS, IN (US); TODD P. GLANCY, FAIRMOUNT, IN (US); TIMOTHY D. HEY, ZIONSVILLE, IN (US); JAMES E. KING, CARMEL, IN (US); RYAN M. LEE, BROWNSBURG, IN (US); JIANQUAN LI, CARY, NC (US); GAOFENG LIN, ZIONSVILLE, IN (US); KRISHNA M. MADDURI, CARMEL, IN (US); THOMAS MEADE, ZIONSVILLE, IN (US); KENNETH NARVA, ZIONSVILLE, IN (US); SEK YEE TAN, LINCOLN, CA (US)

(73) Assignee: CORTEVA AGRISCIENCE LLC, INDIANAPOLIS, IN (US)

(21) Appl. No.: 19/292,159

(22) Filed: Aug. 6, 2025

Related U.S. Application Data
(63) Continuation of application No. 17/933,203, filed on Sep. 19, 2022, now abandoned.
Application No. 17/933,203 is a continuation of application No. 16/780,501, filed on Feb. 3, 2020, now abandoned.
Application No. 16/780,501 is a continuation of application No. 15/541,303, now Pat. No. 10,570,182.
(60) Provisional application No. 62/097,833, filed on Dec. 30, 2014.

(51) Int. Cl. C07K 14/325; A01N 37/46; A01N 63/50; C12N 15/82

(52) U.S. Cl. CPC C07K 14/325; A01N 37/46; A01N 63/50; C12N 15/8286; Y02A 40/146

(57) ABSTRACT

The subject invention concerns Bacillus thuringiensis modified Cry1Ca insecticidal toxins and the polynucleotide sequences which encode these toxins. Uses in transgenic plants are described as are methods for protecting crops from insect pest damage.

### CROSS-REFERENCE TO RELATED APPLICATIONS

This application is a continuation application of U.S. patent application Ser. No. 17/933,203, filed Sep. 19, 2022, which is a continuation application of U.S. patent application Ser. No. 16/780,501, filed Feb. 3, 2020, which is continuation application of U.S. patent application Ser. No. 15/541,303, filed Jun. 30, 2017, now U.S. Pat. No. 10,570,182, which is the U.S. National Phase application of International Patent Application number PCT/US15/066182, filed Dec. 16, 2015, which claims the benefit of U.S. Provisional patent application No. 62/097,833, filed Dec. 30, 2014, the disclosures of each of which are incorporated herein by reference in their entireties.

### REFERENCE TO A SEQUENCE LISTING

The official copy of the sequence listing is submitted electronically via USPTO Patent Center as an XML formatted sequence listing with a file named 110914-US-CON3 created on Aug. 5, 2025 and having a size of 104,259 bytes and is filed concurrently with the specification. The sequence listing comprised in this XML formatted document is part of the specification and is herein incorporated by reference in its entirety.

### FIELD OF THE DISCLOSURE

The subject invention concerns modification of a Bacillus thuringiensis pesticidal toxin, the polynucleotide sequences which encode these toxins and transgenic plants that produce these toxins.

### BACKGROUND OF DISCLOSURE

Insects and other pests cost farmers billions of dollars annually in crop losses and expense to keep these pests under control. In addition to losses in field crops, insect pests are also a burden to vegetable and fruit growers, to producers of ornamental flowers, and to home gardeners. The losses caused by insect pests in agricultural production environments include decrease in crop yield, reduced crop quality, and increased harvesting costs.

Insect pests are mainly controlled by intensive applications of chemical pesticides, which are active through inhibition of insect growth, prevention of insect feeding or reproduction, or cause death. Good insect control can thus be reached, but these chemicals can sometimes affect other beneficial insects. Another problem resulting from the wide use of chemical pesticides is the appearance of resistant insect populations. This has been partially alleviated by various resistance management practices, but there is an increasing need for alternative pest control agents. Biological pest control agents, such as Bacillus thuringiensis (B.t.) strains expressing pesticidal toxins like delta-endotoxins, have also been applied to crop plants with satisfactory results, offering an alternative or compliment to chemical pesticides. The genes coding for some of these delta-endotoxins have been isolated and their expression in heterologous hosts have been shown to provide another tool for the control of economically important insect pests. In particular, the expression of insecticidal toxins, such as Bacillus thuringiensis delta-endotoxins, in transgenic plants have provided efficient protection against selected insect pests, and transgenic plants expressing such toxins have been commercialized, allowing farmers to reduce applications of chemical insect control agents.

Lepidopterans are an important group of agricultural, horticultural, and household pests which cause a large amount of damage each year. This insect order encompasses foliar- and root-feeding larvae and adults. Lepidopteran insect pests include, but are not limited to: Achoroia grisella, Acleris gloverana, Acleris variana, Adoxophyes orana, Agrotis ipsilon (black cutworm “BCW”), Alabama argillacea, Alsophila pometaria, Amyelois transitella, Anagasta kuehniella, Anarsia lineatella, Anisota senatoria, Antheraea pernyi, Anticarsia gemmatalis (velvetbean caterpillar “VBC”), Archips sp., Argyrotaenia sp., Athetis mindara, Bombyx mori, Bucculatrix thurberiella, Cadra cautella, Choristoneura sp., Cochylls hospes, Colias eurytheme, Corcyra cephalonica, Cydia latiferreanus, Cydia pomonella, Datana integerrima, Dendrolimus sibericus, Desmia feneralis, Diaphania hyalinata, Diaphania nitidalis, Diatraea grandiosella (southwestern corn borer “SWCB”), Diatraea saccharalis, Ennomos subsignaria, Eoreuma loftini, Esphestia elutella, Erannis tilaria, Estigmene acrea, Eulia salubricola, Eupocoellia ambiguella, Eupoecilia ambiguella, Euproctis chrysorrhoea, Euxoa messoria, Galleria mellonella, Grapholita molesta, Harrisina americana, Helicoverpa subflexa, Helicoverpa zea (corn earworm “CEW”), Heliothis virescens (tobacco budworm “TBW”), Hemileuca oliviae, Homoeosoma electellum, Hyphantia cunea, Keiferia lycopersicella, Lambdina fiscellaria fiscellaria, Lambdinafiscellaria lugubrosa, Leucoma salicis, Lobesia botrana, Loxostege sticticalis, Lymantria dispar, Macalla thyrisalis, Malacosoma sp., Mamestra brassicae, Mamestra configurata, Manduca quinquemaculata, Manduca sexta, Maruca testulalis, Melanchra picta, Operophtera brumata, Orgyia sp., Ostrinia nubilalis (European corn borer “ECB”), Paleacrita vernata, Papiapema nebris (common stalk borer), Papilio cresphontes, Pectinophora gossypiella, Phryganidia californica, Phyllonorycter blancardella, Pieris napi, Pieris rapae, Plathypena scabra, Platynota flouendana, Platynota stultana, Platyptilia carduidactyla, Plodia interpunctella, Plutella xylostella (diamondback moth “DBM”), Pontia protodice, Pseudaletia unipuncta, Pseudoplusia includens (soybean looper “SBL”), Sabulodes aegrotata, Schizura concinna, Sitotroga cerealella, Spilonta ocellana, Spodoptera eridania (southern armyworm “SAW”), Spodoptera frugiperda (fall armyworm “FAW”), Spodoptera exigua (beet armyworm “BAW”), Thaurnstopoea pityocampa, Ensola bisselliella, Trichoplusia ni (cabbage looper “CL”), Udea rubigalis, Xylomyges curiails, and Yponomeuta padella. Any genus listed above (and others), generally, can also be targeted as a part of the subject invention. Any additional insects in any of these genera (as targets) are also included within the scope of this invention.

Bacillus thuringiensis (B.t.) is a soil-borne, Gram-positive, spore forming bacterium that produces insecticidal crystal proteins known as delta endotoxins or Cry proteins (reviewed in Schnepf et al., 1998). Novel Crystal (Cry) proteins with new insecticidal properties continue to be discovered at an increasing rate, and over 440 Cry genes have been reported. Currently, there are over 450 unique Cry and Cytotoxin (Cyt) proteins classified among 57 primary homology ranks. Cry proteins are named based on the degree of sequence identity, with primary, secondary and tertiary boundaries occurring at approximately 45%, 78% and 95% identity, respectively; close alleles are assigned new quaternary designations (Crickmore et al., 1998). An expansive list of delta endotoxins is maintained and regularly updated at http://www.lifesci.sussex.ac.uk/home/Neil_Crickmore/Bt/intro.html. There are currently over 73 main groups of “Cry” toxins (Cry1-Cry73), with additional Cyt toxins and Vegetative Insecticidal Protein (VIP) toxins and the like. Many of each numeric group have capital-letter subgroups, and the capital letter subgroups have lower-cased letter sub-subgroups. (Cry1 has A-L, and Cry1A has a-i, for example).

B.t. proteins have been used to create the insect-resistant transgenic plants that have been successfully registered or deregulated and commercialized to date. These include Cry1Ab, Cry1Ac, Cry1F, Vip3A, Cry34Ab1/Cry35Ab1, and Cry3Bb in corn, Cry1Ac, Vip3A and Cry2Ab in cotton, and Cry3A in potato. B.t. toxins represent over 90% of the bioinsecticide market and essentially the entire source of genes for transgenic crops that have been developed to provide resistance to insect feeding.

Cry proteins are oral intoxicants that function by acting on midgut cells of susceptible insects. The active forms of many Cry proteins comprise three distinct protein domains. The most well studied B.t. proteins are members of the three-domain Cry delta-endotoxins. These proteins range in size from approximately 70 kDa to 130 kDa. Primary protein sequence analysis reveals five highly conserved sequence blocks and a high degree of sequence variability between conserved blocks three and five (Schnepf et al., 1998).

Three dimensional crystal structures have been determined for Cry1Aa1, Cry2Aa1, Cry3Aa1, Cry3Bb 1, Cry4Aa, Cry4Ba and Cry8Ea1 as examples. These structures are remarkably similar and are comprised of three distinct domains with the following features (reviewed in de Maagd et al., 2003). Domain I is a bundle of seven alpha helices where helix five is surrounded by six amphipathic helices. This domain has been implicated in midgut membrane insertion and pore formation. It shares homology with other pore forming proteins including hemolysins and colicins. Domain II is comprised of three anti-parallel beta sheets packed together in a beta prism. This domain shares homology with certain carbohydrate-binding proteins including vitelline and jacaline. The loops of this domain play important roles in binding insect midgut receptors. In Cry1A proteins, surface exposed loops at the apices of domain II beta sheets are involved in binding to lepidopteran cadherin receptors. Domain III is a beta sandwich structure that interacts with a second class of receptors, examples of which are aminopeptidase and alkaline phosphatase in the case of Cry1A proteins (Piggot and Ellar, 2007). Structurally this domain is related to carbohydrate-binding domains of proteins such as glucanases, galactose oxidase, sialidase and others. This domain binds certain classes of receptor proteins and perhaps participates in insertion of an oligomeric toxin pre-pore. Conserved B.t. sequence blocks 2 and 3 map near the N-terminus and C-terminus of domain 2, respectively. Hence, these conserved sequence blocks 2 and 3 are approximate boundary regions between the three functional domains. These regions of conserved DNA and protein homology have been exploited for engineering recombinant B.t. toxins (U.S. Pat. No. 6,090,931, WO 91/01087, WO95/06730, WO 1998022595).

One proposed model for Cry protein mode of action is based on pore formation in the midgut membranes of susceptible insects (Knowles and Ellar, 1987). In the current version of this model (Bravo et al., 2007), binding to both cadherin and aminopeptidase receptors on Lepidopteran midgut membranes are required for Cry protein toxicity. According to the pore formation model, Cry protein intoxication involves several steps: 1) Proteolytic processing of soluble Cry protoxin to an activated core toxin; 2) Cry protein binding to cadherin receptors on the insect midgut; 3) further proteolytic cleavage at the core toxin N-terminus to remove an α-helical region; 4) Cry protein oligomerization to form a pre-pore; 5) pre-pore binding to second site membrane receptors (aminopeptidases and alkaline phosphatases); 6) pre-pore insertion into the membrane and 7) osmotic cell lysis leading to midgut disruption and insect death.

The widespread adoption of insect-resistant transgenic plant technology gives rise to a concern that pest populations will develop resistance to the insecticidal proteins produced by these plants. Several strategies have been suggested for preserving the utility of B.t.-based insect resistance traits which include deploying proteins at a high dose in combination with a refuge, and alternation with, or co-deployment of, different toxins (McGaughey et al. (1998), “B.t. Resistance Management,” Nature Biotechnol. 16:144-146).

The development of insect resistance to B.t. Cry proteins can result through several mechanisms (Heckel et al., 2007, Piggot and Ellar, 2007). Multiple receptor protein classes for Cry proteins have been identified within insects, and multiple examples exist within each receptor class. Resistance to a particular Cry protein may develop, for example, by means of a mutation within the toxin-binding portion of a cadherin domain of a receptor protein. A further means of resistance may be mediated through a protoxin-processing protease. Thus, resistance to Cry1A toxins in species of Lepidoptera has a complex genetic basis, with at least four distinct, major resistance genes. Lepidopteran insects resistant to Cry proteins have developed in the field for Plutella xylostella (Tabashnik, 1994), Trichoplusia ni (Janmaat and Myers 2003, 2005), Helicoverpa zea (Tabashnik et al., 2008), and Spodoptera frugiperda (Storer, et al., 2010). Development of new high potency Cry proteins will provide additional tools for management of Lepidopteran insect pests.

This invention provides B.t. insecticidal proteins that are effective in controlling insects that are resistant to Cry1Ac and Cry1F. These protein toxins may be used advantageously to protect agronomic crops from insect feeding damage. The ability to express these insect toxins in such a manner that sufficient quantity of the functionally active protein is present in a crop of interest is also a subject of this invention.

### BRIEF SUMMARY OF THE INVENTION

A modified Cry1Ca toxin comprising residues 2 to 68 of SEQ ID NO:2 wherein amino acid residue 54 is chosen from the group consisting of Gly and Ala, amino acid residue 57 is chosen from the group consisting of Leu and Met, and amino acid residue 68 is chosen from the group consisting of Val, Phe, and Ile. A modified Cry1Ca toxin comprising residues 2 to 628 of SEQ ID NO:210 wherein amino acid residue 54 is chosen from the group consisting of Gly and Ala, amino acid residue 57 is chosen from the group consisting of Leu and Met, amino acid residue 68 is chosen from the group consisting of Val, Phe, and Ile, amino acid residue 73 is chosen from the group consisting of Trp, Ala and Met, amino acid residue 596 is chosen from the group consisting of Phe, Met and Ala, and amino acid residue 620 is chosen from the group consisting of Leu and Phe. The modified Cry1Ca toxins of the foregoing further comprising a carboxy terminal extension consisting of amino acid residues 629 to 1164 of SEQ ID NO:36. The modified Cry1Ca toxins of the foregoing further comprising a carboxy terminal extension consisting of amino acid residues 629 to 1164 of SEQ ID NO:36. The modified Cry1Ca toxins of the foregoing further comprising an amino terminal extension consisting of amino acid residues 1 to 74 of SEQ ID NO:40. The modified Cry1Ca toxins of the foregoing further comprising an amino terminal extension consisting of amino acid residues 1 to 74 of SEQ ID NO:40. The modified Cry1Ca toxins of the foregoing further comprising an amino terminal extension consisting of amino acid residues 1 to 74 of SEQ ID NO:40. The modified Cry1Ca toxins of the foregoing further comprising an amino terminal extension consisting of amino acid residues 1 to 74 of SEQ ID NO:40.

DNA encoding modified Cry1Ca toxins, transgenic plants producing modified Cry1Ca toxins and methods of controlling insect pests using the modified Cry1Ca toxins are included in the invention.

The subject invention concerns novel materials and methods for controlling arthropod pests that are detrimental to plants and to agriculture. In a preferred embodiment, the subject invention provides materials and methods for the control of Lepidopteran pests.

Specific B.t. Cry proteins (endotoxins, toxins) useful according to the invention include toxins which can be obtained from the B.t. isolate designated as MR-1206. The subject invention also includes the use of mutants of the exemplified B.t. isolate and toxins which have improved Lepidopteran-active properties, that resist protease processing, or express at high levels when the genes are transformed into a heterologous expression system. Procedures for making mutants are well known in the microbiological art. Ultraviolet light and chemical mutagens such as nitrosoguanidine are used extensively toward this end.

The subject protein toxins can be “applied” or provided to contact the target insects in a variety of ways. For example, transgenic plants (wherein the protein is produced by and present in the plant) can be used and are well-known in the art. Expression of the toxin genes can also be achieved selectively in specific tissues of the plants, such as the roots, leaves, etc. This can be accomplished via the use of tissue-specific promoters, for example. Spray-on applications are another example and are also known in the art. The subject proteins can be appropriately formulated for the desired end use, and then sprayed (or otherwise applied) onto the plant and/or around the plant and/or to the vicinity of the plant to be protected, before an infestation is discovered, after target insects are discovered, both before and after, and the like. The subject protein can also be appropriately formulated and applied to the seeds as a seed treatment that allows the protein to be in contact with the root area of the plant to protect it from root feeding insects. Bait granules, for example, can also be used and are known in the art.

The subject proteins can be used to protect practically any type of plant from damage by a Lepidopteran insect. Examples of such plants include maize, sunflower, soybean, cotton, canola, rice, sorghum, wheat, barley, vegetables, ornamentals, peppers (including hot peppers), sugar beets, fruit, and turf grass, to name but a few. Especially preferred plants are maize, soybean and cotton. A most preferred plant is maize. Another most preferred plant is soybean. Another most preferred plant is cotton.

In one embodiment of the subject invention, the polynucleotide sequences of the subject invention encode toxins of approximately 68-71 kDa. These toxins are used to control Lepidopteran pests, especially fall armyworms, diamondback moths, southwestern corn borer, southern armyworm, corn earworm, and European corn borer. In a preferred embodiment, the subject invention concerns plants cells transformed with at least one polynucleotide sequence of the subject invention such that the transformed plant cells produce and contain pesticidal toxins of the invention in tissues consumed by the target pests.

Alternatively, the B.t. isolate of the subject invention, or recombinant microbes expressing genes encoding the pesticidal toxin proteins described herein, can be used to control insect pests. In this regard, the invention includes the treatment of substantially intact B.t. cells, and/or recombinant cells containing the toxins of the invention, treated to prolong the pesticidal activity when the substantially intact cells are applied to the environment of a target pest. The treated cell acts as a protective coating for the pesticidal toxin. The toxin becomes active upon ingestion by a target insect.

One aspect of the invention pertains to isolated nucleic acid molecules comprising nucleotide sequences encoding pesticidal proteins and polypeptides or biologically active portions thereof, as well as nucleic acid molecules sufficient for use as hybridization probes to identify nucleic acids encoding the claimed toxins. As used herein, the term “nucleic acid molecule” is intended to include DNA molecules (e.g. cDNA or genomic DNA) and RNA molecules (e.g. mRNA) and analogs of the DNA or RNA generated using nucleotide analogs. The nucleic acid molecule can be single-stranded or double-stranded, but preferably is double-stranded DNA.

Nucleotide sequences encoding the proteins of the present invention include the sequences set forth in SEQ ID NOs:1, 3, 5, 7, 9, 11, 13, 15, 17, 19, 21, 23, 25, 27, 29, 31, 33, 35, 37, 39, and complements thereof. By “complement” is intended a nucleotide sequence that is sufficiently complementary to a given nucleotide sequence such that it can hybridize to the given nucleotide sequence to thereby form a stable duplex (double-stranded) molecule. The corresponding amino acid sequences for the pesticidally active modified Cry1Ca toxins encoded by these nucleotide sequences are set forth in SEQ ID NOs:2, 4, 6, 8, 10, 12, 14, 16, 18, 20, 22, 24, 26, 28, 30, 32, 34, 36, 38, and 40.

Nucleic acid molecules that are fragments of the claimed toxin-encoding nucleotide sequences are also encompassed by the present invention. By “fragment” is intended a portion of the nucleotide sequence encoding a fragment of a claimed modified Cry1Ca toxin. A fragment of a nucleotide sequence may encode a biologically active portion of a claimed toxin protein, or it may be a fragment that can be used as a hybridization probe or PCR primer using methods disclosed below.

### BRIEF DESCRIPTION OF THE SEQUENCES

SEQ ID NO:1 DNA sequence encoding DIG-468
SEQ ID NO:2 is the DIG-468 protein sequence
SEQ ID NO:3 DNA sequence encoding DIG-483
SEQ ID NO:4 is the DIG-483 protein sequence
SEQ ID NO:5 DNA sequence encoding DIG-485
SEQ ID NO:6 is the DIG-485 protein sequence
SEQ ID NO:7 DNA sequence encoding DIG-487
SEQ ID NO:8 is the DIG-487 protein sequence
SEQ ID NO:9 DNA sequence encoding DIG-462
SEQ ID NO:10 is the DIG-462 protein sequence
SEQ ID NO:11 DNA sequence encoding DIG-463
SEQ ID NO:12 is the DIG-463 protein sequence
SEQ ID NO:13 DNA sequence encoding DIG-464
SEQ ID NO:14 is the DIG-464 protein sequence
SEQ ID NO:15 DNA sequence encoding DIG-465
SEQ ID NO:16 is the DIG-465 protein sequence
SEQ ID NO:17 DNA sequence encoding DIG-466
SEQ ID NO:18 is the DIG-466 protein sequence
SEQ ID NO:19 DNA sequence encoding DIG-467
SEQ ID NO:20 is the DIG-467 protein sequence
SEQ ID NO:21 DNA sequence encoding DIG-469
SEQ ID NO:22 is the DIG-469 protein sequence
SEQ ID NO:23 DNA sequence encoding DIG-473
SEQ ID NO:24 is the DIG-473 protein sequence
SEQ ID NO:25 DNA sequence encoding DIG-474
SEQ ID NO:26 is the DIG-474 protein sequence
SEQ ID NO:27 DNA sequence encoding DIG-482
SEQ ID NO:28 is the DIG-482 protein sequence
SEQ ID NO:29 DNA sequence encoding modified Cry1Ca codon optimized for maize (IRDIG544.11)
SEQ ID NO:30 is the modified Cry1Ca protein protein toxin sequence (IRDIG544.11)
SEQ ID NO:31 DNA sequence encoding a modified Cry1Ca, IRDIG544.12, with high GC codon optimization
SEQ ID NO:32 protein toxin sequence for IRDIG544.12
SEQ ID NO:33 a dicot optimized DNA sequence encoding modified Cry1Ca, IRDIG544.9
SEQ ID NO:34 protein sequence of modified Cry1Ca, IRDIG544.9
SEQ ID NO:35 a dicot optimized DNA sequence encoding modified Cry1Ca, IRDIG544.8
SEQ ID NO:36 IRDIG544.8 protein codon optimized for dicots
SEQ ID NO:37 DNA sequence encoding a modified Cry1Ca toxin fused to the Cry1Ab protoxin segment
SEQ ID NO:38 is the protein toxin sequence produced from the DNA of SEQ ID NO:37
SEQ ID NO:39 High GC codon optimized DNA sequence encoding a modified Cry1Ca, IRDIG544.12, fused to TraP12
SEQ ID NO:40 Is the modified Cry1Ca toxin, IRDIG544.12, fused toTraP12

### BRIEF DESCRIPTION OF THE FIGURES

FIG. 1 shows expression levels of DIG-465 by construct 115752 and DIG-473 by construct 115753 in T₁ maize leaves sampled by leaf punches.

FIG. 2 is a plot of the amount of leaf damage in maize caused by FAW or Cry1Fa resistant FAW versus the level of expression of DIG-465.

FIG. 3 is a plot of the amount of leaf damage in maize caused by FAW or Cry1Fa resistant FAW versus the level of expression of DIG-473.

### DETAILED DESCRIPTION OF THE INVENTION

By the use of the term “genetic material” herein, it is meant to include all genes, nucleic acid, DNA and RNA. These sequences have been altered in such a manner to increase the stability of the expressed protein toxin when the gene is transformed in a plant, specifically maize and dicots. The protein toxins discussed herein are typically referred to as “insecticides” or “insecticidal”. By insecticides and insecticidal it is meant herein that the protein toxins have a “functional activity” as further defined herein and are used as insect control agents.

By “functional activity” it is meant herein that the protein toxins function as insect control agents in that the proteins are orally active, or have a toxic effect, or are able to disrupt or deter feeding, which may or may not cause death of the insect. When an insect comes into contact with an effective amount of toxin delivered via transgenic plant expression, formulated protein composition(s), sprayable protein composition(s), a bait matrix or other delivery system, the results are typically death of the insect, or the insects do not feed upon the source which makes the toxins available to the insects.

By the use of the term “oligonucleotides” it is meant a macromolecule consisting of a short chain of nucleotides of either RNA or DNA. Such length could be at least one nucleotide, but typically are in the range of about 10 to about 12 nucleotides. The determination of the length of the oligonucleotide is well within the skill of an artisan and should not be a limitation herein. Therefore, oligonucleotides may be less than 10 or greater than 12. The subject invention concerns not only the polynucleotide sequences which encode these classes of toxins, but also the use of these polynucleotide sequences to produce recombinant hosts which express the toxins.

By the use of the term “toxic” or “toxicity” as used herein it is meant that the toxins produced by Bacillus thuringiensis have “functional activity” as defined herein.

By use of the term “modified Cry1Ca toxin(s)” it is meant to include all of the protein sequences of the Sequence Listing and all the variants thereof described herein.

By the use of the term “genetic material” herein, it is meant to include all genes, nucleic acid, DNA and RNA.

For designations of nucleotide residues of polynucleotides, DNA, RNA, oligonucleotides, and primers, and for designations of amino acid residues of proteins, standard IUPAC abbreviations are employed throughout this document. Nucleic acid sequences are presented in the standard 5′ to 3′ direction, and protein sequences are presented in the standard amino (N) terminal to carboxy (C) terminal direction.

The toxins and genes of the subject invention can be further defined by their amino acid and nucleotide sequences, and the sequence of unique fragments comprised by the full-length DNA and amino sequences. The sequences of the molecules within each novel class can be defined herein in terms of homology to certain exemplified sequences as well as in terms of the ability to hybridize with, or be amplified by, certain exemplified probes and primers. The classes of toxins provided herein can also be identified based on their immunoreactivity with certain antibodies.

Toxin structure. The toxin of the subject invention can also be characterized in terms of the structure and domain composition. The correlation of protein sequence variability with differences in bioactivity spectrum led to early hypotheses that the “hypervariable” regions between blocks three and five are responsible for differences in insect specificity among B.t. delta-endotoxins.

When the gene that encodes native full length Cry1Ca protein was inserted and expressed in maize cells, at least 5 detectable proteolytic degradation products were observed. Those five polypeptides were determined to have the following amino acid lengths: 1-1164, 1-628, 29-628, 74-628, and 74-596. Of the five Cry1Ca degradation products detected, two of the fragments were found to be inactive against the key driver insect pests. In most cases, these two inactive fragments represented a major portion of the Cry1Ca-related proteins detected in maize cells. Expressing the native, full length gene for Cry1Ca in maize resulted in plants having insufficient functional activity against key insect pests such as S. frugiperda.

When the gene that expresses truncated native Cry1Ca protein (aa 1-628) was inserted and expressed in maize cells, less proteolytic processing occurred. The majority remained unprocessed and functionally active. Thus expressing the truncated Cry1Ca gene in maize cells resulted in plants having sufficient functional activity against key insect pests due to reduced proteolysis in maize cells.

Altering the primary amino acid sequence of Cry1Ca allows for continual biological activity against key insect pests, and results in less proteolytic processing of the protein, as measured in vitro using chymotrypsin as the protease enzyme. Less proteolytic processing of altered Cry1Ca protein results in higher amounts of functionally active protein accumulating in plants and results in greater activity against the target insect pests.

Protease sensitive variants. Insect gut proteases typically function in aiding the insect in obtaining needed amino acids from dietary protein. The best understood insect digestive proteases are serine proteases, which appear to be the most common type (Englemann and Geraerts, 1980), particularly in Lepidopteran species. Coleopteran insects have guts that are more neutral to acidic than are Lepidopteran guts. The majority of Coleopteran larvae and adults, for example Colorado potato beetle, have slightly acidic midguts, and cysteine proteases provide the major proteolytic activity (Wolfson and Murdock, 1990). More precisely, Thie and Houseman (1990) identified and characterized the cysteine proteases, cathepsin B-like and cathepsin H-like, and the aspartyl protease, cathepsin D-like, in Colorado potato beetle. Gillikin et al., (1992) characterized the proteolytic activity in the guts of western corn rootworm larvae and found primarily cysteine proteases. U.S. Pat. No. 7,230,167 disclosed that the serine protease, cathepsin G, exists in western corn rootworm. The diversity and different activity levels of the insect gut proteases may influence an insect's sensitivity to a particular B.t. toxin.

In one embodiment, the toxins have specific changes in their amino acid sequences that significantly reduce the level of protease processing of the expressed protein by proteases found naturally in maize plants. The changes in amino acids results in higher levels of functional activity of the protein when expressed in maize. Protease cleavage sites may be introduced at desired locations by chemical gene synthesis or splice overlap PCR (Horton et al., 1989). Serine protease recognition sequences, for example, can optionally be inserted at specific sites in the Cry protein structure to affect protein processing at desired deletion points within the midgut of certain insect pests. Lepidopteran midgut serine proteases such as trypsin or trypsin-like enzymes, chymotrypsin, elastase, etc. (Christeller et al., 1992) can be exploited for activation of Cry proteins by engineering protease recognition sequences at desired processing sites. Likewise, Coleopteran serine proteases such as trypsin, chymotrypsin and cathepsin G-like protease may similarly be exploited by engineering recognition sequences at desired processing sites. Further, Coleopteran cysteine proteases such as cathepsins (B-like, L-like, O-like, and K-like proteases) (Koiwa et al., 2000 and Bown et al., 2004), metalloproteases such as ADAM10 (Ochoa-Campuzano et al., 2007), and aspartic acid proteases such as cathepsins D-like and E-like, pepsin, plasmepsin, and chymosin may be exploited by engineering recognition sequences at desired processing sites.

The scope of this invention includes variant Cry1Ca insecticidal proteins that are produced by manipulating the encoding sequence for the subject insecticidal proteins by introduction or elimination of protease processing sites at appropriate positions to allow, or eliminate, proteolytic cleavage of a larger variant protein by insect, plant, or microorganism proteases. The end result of such manipulation is the generation of toxin molecules having the same or better activity as the intact (full length) native toxin protein.

Unlike the high sequence specificity associated with Type II restriction endonucleases in the recognition and cleavage of their DNA substrates, proteolytic enzymes are more nonspecific in the amino sequence comprising the cleavage recognition site. Some generalities have been discovered regarding the amino acid structures comprising some protease cleavage sites, in particular, cathepsin G as compared to cathepsins B, K, L, and S (Bown et al., 2004). In the nomenclature of protease cleavage sites in the illustrations below, the amino acid residues upstream (i.e. towards the N-terminus) from the cleavage site are numbered P1, P2, P3, P4, P5, etc, with residue P1 being immediately adjacent to the cleavage site, and residue P5 being the fifth most distal from the cleavage site in the N-terminal direction. Amino acid residues downstream (i.e. towards the C-terminus) from the cleavage site are numbered P1′, P2′, P3′, P4′, P5′ etc., with residue P1′ being immediately adjacent to the cleavage site, and residue P5′ being the fifth most distal from the cleavage site in the C-terminal direction. Cathepsin G is known to exhibit preferential cleavage after P1 residues glutamine, lysine, tryptophan, or phenylalanine, where residues P2, P3, P4, P5, etc., and P1′, P2′, P3′, P4′ P5′, etc. can be any of the 20 amino acids normally found in natural proteins. Somewhat enhanced cleavage site sequence specificity is demonstrated by cathepsins B, K, L, and S, wherein the side chain of the P2 amino acid fits into a substrate binding site S2 of the cathepsin. The S2 site of these cathepsins preferentially interacts with P2 amino acids having large hydrophobic side chains (e.g. as found in valine, leucine, isoleucine, phenylalanine, tryptophan, and tyrosine), and disfavors interaction with P2 residues having charged side chains (except that cathepsins B and L accept the large hydrophilic charged side chain of arginine in the P2 position). Some specificity is seen in the identity of the amino acid in the P3 position. For example, cathepsin L cleaves preferentially after arginine in the P1 position, when phenylalanine or arginine occupy the P2 position. The P3 amino acid can be either an aromatic type (e.g. phenylalanine, tryptophan, histidine, or tyrosine) or a hydrophobic type (e.g. alanine, valine, leucine, isoleucine, phenylalanine, tryptophan, or tyrosine). Positions P4, P5, etc. and P1′, P2′, P3′, P4′, P5′, etc. can be any of the 20 amino acids normally found in natural proteins.

Proteolytic cleavage is further dependent on the availability of the subject cleavage sequence to the respective protease; sequestration of the potential cleavage site within the three-dimensional structure of the protein may render the protein resistant to cleavage by the particular protease. It is thought that the diversity and different activity levels of the insect gut proteases may influence an insect's sensitivity to a particular B.t. toxin. One skilled in the arts of biochemistry and molecular biology can examine the biochemical characteristics (including, but not limited to, determination of the sequences of the amino acids comprising the N-terminus and C-terminus of the polypeptide) of insecticidal protein fragments generated by protease cleavage/activation of larger proteins by the gut proteases of susceptible insects. One may also characterize the protease regime of the guts of nonsusceptible insects or host plants, and engineer, at appropriate places within the coding sequence for the B.t. insecticidal protein, sequences amenable to cleavage by the gut proteases of nonsusceptible insects or prospective host plants in which the B.t. insecticidal protein will be produced transgenically. Such analyses and manipulations of the subject B.t. insecticidal protein are understood to be within the scope of this invention.

In another embodiment, the toxins have specific changes in their amino acid sequences that significantly enhance the level of protein expression, when expressed in a variety of different expression systems, including plant and bacteria. The result of the increased expression of the protein is increased functional activity in the expression system. This is advantageous in providing a high dose of the toxin to the insect which can prevent the occurrence of resistance in the insects to the toxins due to survival of small populations of insects receiving a sub lethal dose of the protein toxin.

Genes and toxins. The protein molecules of the embodiments herein comprise amino acid sequences that are homologous to known pesticidal proteins, particularly B.t. Cry proteins, more particularly Cry1Ca protein (Genbank Accession No. AAA22343). The predicted amino acid sequences encoded by a nucleotide sequences of the embodiments are disclosed as SEQ ID NOs:2, 4, 6, 8, 10, 12, 14, 16, 18, 20, 22, 24, 26, 28, 30, 32, 34, 36, 38, and 40.

The sequence of toxins of the subject invention are provided as SEQ ID NOs:2, 4, 6, 8, 10, 12, 14, 16, 18, 20, 22, 24, 26, 28, 30, 32, 34, 36, 38, and 40. In a preferred embodiment, the toxins of the subject invention have at least one of the following characteristics:
(a) said toxin is encoded by a nucleotide sequence which hybridizes under stringent conditions with a nucleotide sequence selected from the group consisting of SEQ ID NOs:1, 3, 5, 7, 9, 11, 13, 15, 17, 19, 21, 23, 25, 27, 29, 31, 33, 35, 37, 39, or their complementary sequences.
(b) said toxin is immunoreactive with an antibody raised against an approximately 68-71 kDa pesticidal toxin, or a fragment thereof, from a Bacillus thuringiensis isolate.
(c) said toxin is encoded by a nucleotide sequence wherein a portion of said nucleotide sequence can be amplified by PCR using a primer pair to produce a fragment of about 25-40 bp,
(d) said toxin comprises a pesticidal portion of the amino acid sequences shown in SEQ ID NOs:2, 4, 6, 8, 10, 12, 14, 16, 18, 20, 22, 24, 26, 28, 30, 32, 34, 36, 38, and 40,
(e) said toxin comprises an amino acid sequence which has at least about (90%) homology with a pesticidal portion of an amino acid sequence selected from the group consisting of SEQ ID NOs:2, 4, 6, 8, 10, 12, 14, 16, 18, 20, 22, 24, 26, 28, 30, 32, 34, 36, 38, and 40,
(f) said toxin is encoded by a nucleotide sequence which hybridizes under stringent conditions with an insecticidal portion of a nucleotide sequence selected from the group consisting of DNA which encodes SEQ ID NOs:2, 4, 6, 8, 10, 12, 14, 16, 18, 20, 22, 24, 26, 28, 30, 32, 34, 36, 38, and 40,
(g) said toxin is immunoreactive with an antibody to an approximately 68 kDa or 130 kDa pesticidal toxin, or a fragment thereof, from a Bacillus thuringiensis isolate, MR-1206.
(h) said toxin comprises an amino acid sequence which has at least about (90%) homology with an amino acid sequence selected from the group consisting of SEQ ID NOs:2, 4, 6, 8, 10, 12, 14, 16, 18, 20, 22, 24, 26, 28, 30, 32, 34, 36, 38, and 40 and pesticidal portions of SEQ ID NOs:2, 4, 6, 8, 10, 12, 14, 16, 18, 20, 22, 24, 26, 28, 30, 32, 34, 36, 38, and 40.

The specific genes exemplified herein, variations of these genes, and fragments of these genes may also be obtained, for example, by synthetic construction by methods currently practiced by any of several commercial suppliers (see for example, U.S. Pat. No. 7,482,119). These genes, or portions or variants thereof, may also be constructed synthetically, for example, by use of a gene synthesizer and the methods of, for example, U.S. Pat. No. 5,380,831. Alternatively, variations of synthetic or naturally occurring genes may be readily constructed using standard molecular biological techniques for making point mutations. Fragments of these genes can also be made using commercially available exonucleases or endonucleases according to standard procedures. For example, enzymes such as Bal31 or site-directed mutagenesis can be used to systematically cut off nucleotides from the ends of these genes. Also, gene fragments which encode active toxin fragments may be obtained using a variety of restriction enzymes.

Nucleic acid molecules that are fragments of a claimed toxin-encoding nucleotide sequence comprise at least about 15, 20, 30, 40, 50, 60, 75, 100, 200, 300, 350, 400, 450, 500, 550, 600, 650, 700, 750, 800, 850, 900, 950, 1000, 1100, 1200, 1300, 1400, 1500, 1600, 1700, 1800, 1900, 2000, 2100, 2200, 2300, 2400, 2500, 3000, 3500 nucleotides, or up to the number of nucleotides present in a full-length claimed insecticidal toxin-encoding nucleotide sequence disclosed herein (for example, 1,878 nucleotides for SEQ ID NO:1; 3,495 nucleotides for SEQ ID NO:37), depending upon the intended use. A fragment of a nucleotide sequence that encodes a biologically active portion of a claimed protein of the invention will encode at least about 15, 25, 30, 40, 50, 75, 100, 125, 150, 175, 200, 250, 300, 350, 400, 450, 500, 550, 600, 650, 700, 800, 900, 1000, 1100 or 1200 contiguous amino acids, or up to the total number of amino acids present in a full-length insecticidal protein of the invention (for example, 625 amino acids for SEQ ID NO:2 or 1,164 amino acids for SEQ ID NO:38).

Recombinant hosts. The toxin-encoding genes of the subject invention can be introduced into a wide variety of microbial or plant hosts. Expression of the toxin gene results, directly or indirectly, in the intracellular production and maintenance of the pesticidal protein. With suitable microbial hosts, e.g. Pseudomonas, the microbes can be applied to the environment of the pest, where they will proliferate and can be ingested. The result is control of the pest. Alternatively, the microbe hosting the toxin gene can be treated under conditions that prolong the activity of the toxin and stabilize the cell. The treated cell, which retains the toxic activity, then can be applied to the environment of the target pest.

Where the toxin gene is introduced via a suitable vector into a microbial host, and said host is applied to the environment in a living state, it is essential that certain host microbes be used. Microorganism hosts are selected which are known to occupy the “phytosphere” (phylloplane, phyllosphere, rhizosphere, and/or rhizoplane) of one or more crops of interest. These microorganisms are selected so as to be capable of successfully competing in the particular environment (crop and other insect habitats) with the wild-type indigenous microorganisms, provide for stable maintenance and expression of the gene expressing the polypeptide pesticide, and, desirably, provide for improved protection of the pesticide from environmental degradation and inactivation.

B.t. spores or recombinant host cells also can be treated prior to being applied or formulated for application to plants. For example, isolated B.t. spores and/or toxin crystals can be chemically treated to prolong insecticidal activity and thereby include a treated polypeptide of the invention (U.S. Pat. No. 4,695,462 and Gaertner et al., 1993).

A large number of microorganisms are known to inhabit the phylloplane (the surface of the plant leaves) and/or the rhizosphere (the soil surrounding plant roots) of a wide variety of important crops. These microorganisms include bacteria, algae, and fungi. Of particular interest are microorganisms, such as bacteria, e.g. genera Pseudomonas, Erwinia, Serratia, Klebsiella, Xanthomonas, Streptomyces, Rhizobium, Sinorhizobium, Rhodopseudomonas, Methylophilius, Agrobacterium, Acetobacter, Lactobacillus, Arthrobacter, Azotobacter, Leuconostoc, and Alcaligenes; and fungi, particularly yeast, e.g. genera Saccharomyces, Cryptococcus, Kluyveromyces, Sporobolomyces, Rhodotorula, and Aureobasidium. Of particular interest are such phytosphere bacterial species as Pseudomonas syringae, Pseudomonas fluorescens, Serratia marcescens, Acetobacter xylinum, Agrobacterium tumefaciens, Agrobacterium radiobacter, Rhodopseudomonas spheroides, Xanthomonas campestris, Sinorhizobium meliloti (formerly Rhizobium meliloti), Alcaligenes eutrophus, and Azotobacter vinelandii; and phytosphere yeast species such as Rhodotorula rubra, R. glutinis, R. marina, R. aurantiaca, Cryptococcus albidus, C. diffluens, C. laurentii, Saccharomyces rosei, S. pretoriensis, S. cerevisiae, Sporobolomyces roseus, S. odorus, Kluyveromyces veronae, and Aureobasidium pollulans. Of particular interest are the pigmented microorganisms.

A preferred embodiment of the subject invention is the transformation of plants with genes encoding the subject insecticidal protein or its variants. The transformed plants are resistant to attack by an insect target pest by virtue of the presence of controlling amounts of the subject insecticidal protein or its variants in the cells of the transformed plant. By incorporating genetic material that encodes the insecticidal properties of the B.t. insecticidal toxins into the genome of a plant eaten by a particular insect pest, the adult or larvae would die after consuming the food plant. Numerous members of the monocotyledonous and dicotyledonous classifications have been transformed. Transgenic agronomic crops as well as fruits and vegetables are of commercial interest. Such crops include but are not limited to maize, rice, soybeans, canola, sunflower, alfalfa, sorghum, wheat, cotton, peanuts, tomatoes, potatoes, and the like. Several techniques exist for introducing foreign genetic material into monocot or dicot plant cells, and for obtaining fertile plants that stably maintain and express the introduced gene. Such techniques include acceleration of genetic material coated onto microparticles directly into cells (U.S. Pat. Nos. 4,945,050 and 5,141,131). Plants may be transformed using Agrobacterium technology, see U.S. Pat. Nos. 5,177,010, 5,104,310, European Patent Application 0131624B1, European Patent Application 120516, European Patent Application 159418B, European Patent Application 176112, U.S. Pat. Nos. 5,149,645, 5,469,976, 5,464,763, 4,940,838, 4,693,976, European Patent Application 116718, European Patent Application 290799, European Patent Application 320500, European Patent Application 604662, European Patent Application 627752, European Patent Application 0267159, European Patent Application 0292435, U.S. Pat. Nos. 5,231,019, 5,463,174, 4,762,785, 5,004,863, and 5,159,135. Other transformation technology includes WHISKERS™ technology, see U.S. Pat. Nos. 5,302,523 and 5,464,765. Electroporation technology has also been used to transform plants, see WO 87/06614, U.S. Pat. Nos. 5,472,869, 5,384,253, WO9209696, and WO9321335. All of these transformation patents and publications are incorporated by reference. In addition to numerous technologies for transforming plants, the type of tissue which is contacted with the foreign genes may vary as well. Such tissue would include but would not be limited to embryogenic tissue, callus tissue type I and II, hypocotyl, meristem, and the like. Almost all plant tissues may be transformed during dedifferentiation using appropriate techniques within the skill of an artisan.

Genes encoding modified Cry1Ca insecticidal toxins and variants can be inserted into plant cells using a variety of techniques which are well known in the art as disclosed above. For example, a large number of cloning vectors comprising a marker that permits selection of the transformed microbial cells and a replication system functional in E. coli are available for preparation and modification of foreign genes for insertion into higher plants. Such manipulations may include, for example, the insertion of mutations, truncations, additions, deletions, or substitutions as desired for the intended use. The vectors comprise, for example, pBR322, pUC series, M13mp series, pACYC184, etc. Accordingly, the sequence encoding the Cry protein or variants can be inserted into the vector at a suitable restriction site. The resulting plasmid is used for transformation of E. coli, the cells of which are cultivated in a suitable nutrient medium, then harvested and lysed so that workable quantities of the plasmid are recovered. Sequence analysis, restriction fragment analysis, electrophoresis, and other biochemical-molecular biological methods are generally carried out as methods of analysis. After each manipulation, the DNA sequence used can be cleaved and joined to the next DNA sequence. Each manipulated DNA sequence can be cloned in the same or other plasmids.

Depending on the plant transformation method, ancillary DNA sequences may be necessary. If, for example, a Ti or Ri plasmid is used for the transformation of the plant cell, then at least a T-DNA right border repeat, but often both the right border repeat and the left border repeat of the Ti or Ri plasmid will be joined as the flanking region of the genes desired to be inserted into the plant cell. The use of T-DNA-containing vectors for the transformation of plant cells has been intensively researched and sufficiently described in EP 120516; Lee and Gelvin (2008), Fraley et al., (1986), and An et al., (1985), and is well established in the field.

Once the inserted DNA has been integrated into the plant genome, it is relatively stable throughout subsequent generations. The vector used to transform the plant cell normally contains a selectable marker gene encoding a protein that confers on the transformed plant cells tolerance to a herbicide or an antibiotic, such as bialaphos, kanamycin, G418, bleomycin, or hygromycin, inter alia. The individually employed selectable marker gene should accordingly permit the selection of transformed cells while the growth of cells that do not contain the inserted DNA is suppressed by the selective compound.

A large number of techniques are available for inserting DNA into a host plant cell. Those techniques include transformation with T-DNA delivered by Agrobacterium tumefaciens or Agrobacterium rhizogenes as the transformation agent. Additionally, fusion of plant protoplasts with liposomes containing the DNA to be delivered, direct injection of the DNA, biolistics transformation (microparticle bombardment), or electroporation, as well as other possible methods, may be employed. One skilled in the field of plant transformation will understand that multiple methodologies are available for the production of transformed plants, and they may be modified and specialized to accommodate biological differences between various host plant species.

If Agrobacterium strains are used for the transformation, the DNA to be inserted will be cloned into special plasmids, namely either into an intermediate (shuttle) vector or into a binary vector. The intermediate vectors can be integrated into the Ti or Ri plasmid or derivatives thereof by homologous recombination owing to sequences that are homologous between the Ti or Ri plasmid and the intermediate plasmid. The Ti or Ri plasmid also comprises the vir region comprising vir genes necessary for the transfer of the T-DNA. Intermediate vectors cannot replicate in Agrobacteria. The intermediate vector can be transferred into Agrobacterium tumefaciens by means of a helper plasmid (via bacterial conjugation), by electroporation, by direct DNA, chemically mediated transformation, or by other methodologies. Binary vectors can replicate autonomously in both E. coli and Agrobacterium cells. They comprise sequences, framed by the right and left T-DNA border repeat regions, that may include a selectable marker gene functional for the selection of transformed plant cells, a cloning linker, cloning polylinker, or other sequence which can function as an introduction site for genes destined for plant cell transformation. They can be transformed directly into Agrobacterium cells (Holsters et al., (1978)) by electroporation, or by direct DNA, chemically mediated transformation, or introduced by bacterial conjugation, or by other methodologies. The Agrobacterium used as host cell is to comprise a plasmid carrying a vir region. The vir region is necessary for the transfer of the T-DNA into the plant cell. Additional T-DNA regions additive to the one containing the gene encoding the B.t. insecticidal toxin protein or its variants may be present in the Agrobacterium host cell. The bacterium cells so transformed are used for the transformation of plant cells. Plant explants (for example, pieces of leaf, segments of stalk, roots, but also protoplasts or suspension-cultivated cells) can advantageously be cultivated with Agrobacterium tumefaciens or Agrobacterium rhizogenes for the transfer of the DNA into the plant cell. Whole plants may then be regenerated from the infected plant material following placement in suitable growth conditions and culture medium, which may contain antibiotics or herbicides for selection of the transformed plant cells. The plants so obtained can then be tested for the presence of the inserted DNA.

The transformed cells grow inside the plants in the usual manner. They can form germ cells and transmit the transformed trait(s) to progeny plants. Such plants can be grown in the normal manner and crossed with plants that have the same transformed hereditary factors or other hereditary factors. The resulting hybrid individuals have the corresponding phenotypic properties, for example, the ability to control the feeding of plant pest insects.

No special demands are made in the construction of the plasmids in the case of those used for injection and electroporation. It is possible to use ordinary plasmids, such as, for example, pUC derivatives appropriately modified to contain all the genes desired to be transferred to plant cells.

The activity of recombinant polynucleotides inserted into plant cells can be dependent upon the influence of endogenous plant DNA adjacent to the insert. Thus, another option is to take advantage of events that are known to be excellent locations in a plant genome for insertions. See e.g. WO 2005/103266 A1, relating to Cry1F and Cry1Ac cotton events; the subject B.t. insecticidal toxin gene can be substituted in those genomic loci in place of the Cry1F or Cry1Ac inserts. Targeted homologous recombination, for example, can be used according to the subject invention. This type of technology is the subject of, for example, WO 03/080809 and the corresponding published U.S. application (USPA 20030232410), relating to the use of zinc fingers for targeted recombination. The use of recombinases (cre-lox and flp-frt for example) is also known in the art.

In a preferred embodiment of the subject invention, plants will be transformed with genes wherein the codon usage of the protein coding region has been optimized for plants. See, for example, U.S. Pat. No. 5,380,831, which is hereby incorporated by reference. Also, advantageously, plants encoding a truncated toxin will be used. The truncated toxin typically will encode about 55% to about 80% of the full length toxin. Methods for creating synthetic B.t. genes for use in plants are known in the art (Stewart 2007).

Another variable is the choice of a selectable marker. The preference for a particular marker is at the discretion of the artisan, but any of the following selectable markers may be used along with any other gene not listed herein which could function as a selectable marker. Such selectable markers include but are not limited to aminoglycoside phosphotransferase gene of transposon Tn5 (Aph II) which encodes resistance to the antibiotics kanamycin, neomycin and G418, as well as those genes which code for tolerance to glyphosate; hygromycin; methotrexate; phosphinothricin (bialaphos); imidazolinones, sulfonylureas and triazolopyrimidine herbicides, such as chlorosulfuron; bromoxynil, dalapon and the like. Examples of such genes are provided in Merlo, (2002), which is incorporated herein by reference.

In addition to a selectable marker, it may be desirable to use a reporter gene. In some instances a reporter gene may be used without a selectable marker. Reporter genes are genes which typically do not provide a growth advantage to the recipient organism or tissue. The reporter gene typically encodes for a protein which provides for some phenotypic change or enzymatic property. A preferred reporter gene is the glucuronidase (GUS) gene. Other examples of reporter genes are provided in Merlo (2002).

Regardless of transformation technique, the gene is preferably incorporated into a gene transfer vector adapted to express the B.t insecticidal toxin genes and variants in the plant cell by including in the vector a plant promoter. In addition to plant promoters, promoters from a variety of sources can be used efficiently in plant cells to express foreign genes. For example, promoters of bacterial origin, such as the octopine synthase promoter, the nopaline synthase promoter, the mannopine synthase promoter; promoters of viral origin, such as the 35S and 19S promoters of cauliflower mosaic virus, and the like may be used. Plant promoters include, but are not limited to ribulose-1,6-bisphosphate (RUBP) carboxylase small subunit (ssu), beta-conglycinin promoter, phaseolin promoter, ADH (alcohol dehydrogenase) promoter, heat-shock promoters, ADF (actin depolymerization factor) promoter, and tissue specific promoters. Promoters may also contain certain enhancer sequence elements that may improve the transcription efficiency. Typical enhancers include but are not limited to ADH1-intron 1 and ADH1-intron 6. Constitutive promoters may be used. Constitutive promoters direct continuous gene expression in nearly all cells types and at nearly all times (e.g., actin, ubiquitin, CaMV 35S). Tissue specific promoters are responsible for gene expression in specific cell or tissue types, such as the leaves or seeds (e.g., zein, oleosin, napin, ACP (Acyl Carrier Protein)), and these promoters may also be used. Promoters may also be used that are active during a certain stage of the plants' development as well as active in specific plant tissues and organs. Examples of such promoters include but are not limited to promoters that are root specific, pollen-specific, embryo specific, corn silk specific, cotton fiber specific, seed endosperm specific, phloem specific, and the like.

Under certain circumstances it may be desirable to use an inducible promoter. An inducible promoter is responsible for expression of genes in response to a specific signal, such as: physical stimulus (e.g. heat shock genes); light (e.g. RUBP carboxylase); hormone (e.g. glucocorticoid); antibiotic (e.g. tetracycline); metabolites; and stress (e.g. drought). Other desirable transcription and translation elements that function in plants may be used, such as 5′ untranslated leader sequences, RNA transcription termination sequences and poly-adenylate addition signal sequences. Numerous plant-specific gene transfer vectors are known in the art.

The subject invention includes plant cells that are not totipotent (non-totipotent), plant cells that are not propagative material (for example, leaf cells in some embodiments; seed cells are excluded from some embodiments) and are incapable of differentiating into whole plants. The subject invention includes plant cells that have uses other than for regenerating into a whole plant. For example, said plant cells can be used to produce a protein (such as a DIG-465 protein of the subject invention). Thus, plant cells of the subject invention include those that have uses other than totipotency (that is, some cells of subject invention are not regenerable into a whole plant). However, some embodiments do include seed cells and plant cells that can be regenerated into a whole plant.

A further method for identifying the toxins and genes of the subject invention is through the use of oligonucleotide probes. These probes are detectable nucleotide sequences. These sequences may be rendered detectable by virtue of an appropriate radioactive label or may be made inherently fluorescent as described in U.S. Pat. No. 6,268,132. As is well known in the art, if the probe molecule and nucleic acid sample hybridize by forming strong base-pairing bonds between the two molecules, it can be reasonably assumed that the probe and sample have substantial sequence homology. Preferably, hybridization is conducted under stringent conditions by techniques well-known in the art, as described, for example, in Keller and Manak (1993). Detection of the probe provides a means for determining in a known manner whether hybridization has occurred. Such a probe analysis provides a rapid method for identifying toxin-encoding genes of the subject invention. The nucleotide segments which are used as probes according to the invention can be synthesized using a DNA synthesizer and standard procedures. These nucleotide sequences can also be used as PCR primers to amplify genes of the subject invention.

As used herein the terms “stringent conditions” or “stringent hybridization conditions” are intended to refer to conditions under which a probe will hybridize (anneal) to its target sequence to a detectably greater degree than to other sequences (e.g. at least 2-fold over background). Stringent conditions are sequence-dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, a probe is less than about 1000 nucleotides in length, preferably less than 500 nucleotides in length.

Typically, stringent conditions will be those in which the salt concentration is less than about 1.5 M Na ion, typically about 0.01 to 1.0 M Na ion concentration (or other salts) at pH 7.0 to pH 8.3 and the temperature is at least about 30° C. for short probes (e.g. 10 to 50 nucleotides) and at least about 60° C. for long probes (e.g. greater than 50 nucleotides). Stringent conditions may also be achieved with the addition of destabilizing agents such as formamide. Exemplary low stringency conditions include hybridization with a buffer solution of 30% to 35% formamide, 1 M NaCl, 1% SDS (sodium dodecyl sulfate) at 37° C. and a wash in 1× to 2×SSC (20×SSC=3.0 M NaCl/0.3 M trisodium citrate) at 50° C. to 55° C. Exemplary moderate stringency conditions include hybridization in 40% to 45% formamide, 1.0 M NaCl, 1% SDS at 37° C. and a wash in 0.5× to 1×SSC at 55° C. to 60° C. Exemplary high stringency conditions include hybridization in 50% formamide, 1 M NaCl, 1% SDS at 37° C. and awash in 0.1×SSC at 60° C. to 65° C. Optionally, wash buffers may comprise about 0.1% to about 1% SDS. Duration of hybridization is generally less than about 24 hours, usually about 4 to about 12 hours.

Specificity is typically the function of post-hybridization washes, the critical factors being the ionic strength and temperature of the final wash solution. For DNA/DNA hybrids, the thermal melting point (Tm) is the temperature (under defined ionic strength and pH) at which 50% of a complementary target sequence hybridizes to a perfectly matched probe. Tm is reduced by about 1° C. for each 1% of mismatching; thus, Tm, hybridization conditions, and/or wash conditions can be adjusted to facilitate annealing of sequences of the desired identity. For example, if sequences with >90% identity are sought, the Tm can be decreased 10° C. Generally, stringent conditions are selected to be about 5° C. lower than the Tm for the specific sequence and its complement at a defined ionic strength and pH. However, highly stringent conditions can utilize a hybridization and/or wash at 1° C., 2° C., 3° C., or 4° C. lower than the Tm; moderately stringent conditions can utilize a hybridization and/or wash at 6° C., 7° C., 8° C., 9° C., or 10° C. lower than the Tm, and low stringency conditions can utilize a hybridization and/or wash at 11° C., 12° C., 13° C., 14° C., 15° C., or 20° C. lower than the Tm.

Tm (in ° C.) may be experimentally determined or may be approximated by calculation. For DNA-DNA hybrids, the Tm can be approximated from the equation of Meinkoth and Wahl (1984):

$$Tm ( °C . ) = 81.5 °C . + 16.6 ( log M ) + 0.41 ( \% G C ) - 0.61 ( \% formamide ) - 500 / L ;$$

where M is the molarity of monovalent cations, % GC is the percentage of guanosine and cytosine nucleotides in the DNA, % formamide is the percentage of formamide in the hybridization solution, and L is the length of the hybrid in base pairs

Alternatively, the Tm is described by the following formula (Beltz et al., 1983).

$$Tm ( °C . ) = 81.5 °C . + 16.6 ( log [ Na + ] ) + 0.41 ( \% G C ) - 0.61 ( \% formamide ) - 600 / L$$

where [Na+] is the molarity of sodium ions, % GC is the percentage of guanosine and cytosine nucleotides in the DNA, % formamide is the percentage of formamide in the hybridization solution, and L is the length of the hybrid in base pairs

Using the equations, hybridization and wash compositions, and desired Tm, those of ordinary skill will understand that variations in the stringency of hybridization and/or wash solutions are inherently described. If the desired degree of mismatching results in a Tm of less than 45° C. (aqueous solution) or 32° C. (formamide solution), it is preferred to increase the SSC concentration so that a higher temperature can be used. An extensive guide to the hybridization of nucleic acids is found in Tijssen (1993) and Ausubel et al., (1995). Also see Sambrook et al., (1989).

Hybridization of immobilized DNA on Southern blots with radioactively labeled gene-specific probes may be performed by standard methods (Sambrook et al., supra). Radioactive isotopes used for labeling polynucleotide probes may include 32P, 33P, 14C, or 3H. Incorporation of radioactive isotopes into polynucleotide probe molecules may be done by any of several methods well known to those skilled in the field of molecular biology. (See, e.g. Sambrook et al., supra.) In general, hybridization and subsequent washes may be carried out under stringent conditions that allow for detection of target sequences with homology to the claimed toxin encoding genes. For double-stranded DNA gene probes, hybridization may be carried out overnight at 20-25° C. below the Tm of the DNA hybrid in 6×SSPE, 5×Denhardt's Solution, 0.1% SDS, 0.1 mg/mL denatured DNA [20×SSPE is 3M NaCl, 0.2 M NaHPO4, and 0.02M EDTA (ethylenediamine tetra-acetic acid sodium salt); 100×Denhardt's Solution is 20 gm/L Polyvinylpyrollidone, 20 gm/L Ficoll type 400 and 20 gm/L Bovine Serum Albumin (fraction V)].

Washes may typically be carried out as follows:
(1) Twice at room temperature for 15 minutes in 1×SSPE, 0.1% SDS (low stringency wash).
(2) Once at Tm −20° C. for 15 minutes in 0.2×SSPE, 0.1% SDS (moderate stringency wash).

For oligonucleotide probes, hybridization may be carried out overnight at 10-20° C. below the Tm of the hybrid in 6×SSPE, 5×Denhardt's solution, 0.1% SDS, 0.1 mg/mL denatured DNA. Tm for oligonucleotide probes may be determined by the following formula (Suggs et al., 1981).

$$Tm ( °C . ) = 2 ( number of T / A base pairs ) + 4 ( number of G / C base pairs )$$

Washes may typically be carried out as follows:
(1) Twice at room temperature for 15 minutes 1×SSPE, 0.1% SDS (low stringency wash).
(2) Once at the hybridization temperature for 15 minutes in 1×SSPE, 0.1% SDS (moderate stringency wash).

A practitioner skilled in the art will realize that probe molecules for hybridization and hybrid molecules formed between probe and target molecules may be rendered detectable by means other than radioactive labeling.

Variant toxins. The genes and toxins useful according to the subject invention include not only the truncated sequences disclosed but also full length sequences, fragments of these sequences, variants, mutants, and fusion proteins which retain the characteristic pesticidal activity of the toxins specifically exemplified herein. As used herein, the terms “variants” or “variations” of genes refer to nucleotide sequences which encode the same toxins or which encode equivalent toxins having pesticidal activity. Further as used herein, the term “equivalent toxins” refers to toxins having the same or essentially the same biological activity against the target pests as the claimed toxins. Thus, the variant or variations of the claimed toxins will have at least about 30%, preferably at least about 50%, more preferably at least about 70%, even more preferably at least about 80% of the activity of the claimed toxins. Methods for measuring pesticidal activity are well known in the art and are exemplified herein. By “variants” it is intended herein to include proteins or polypeptides having an amino acid sequence that is at least about 60%, 65%, preferably about 70%, 75%, more preferably about 80%, 85%, most preferably about 90%, 91%, 92%, 93%, 94%, 95%, 96%, 97%, 98% or 99% identical to the amino acid sequence of SEQ ID NOs:2, 4, 6, 8, 10, 12, 14, 16, 18, 20, 22, 24, 26, 28, 30, 32, 34, 36, 38, and 40. Variants also include polypeptides encoded by a nucleic acid molecule that hybridizes to the nucleic acid molecule of SEQ ID NOs:1, 3, 5, 7, 9, 11, 13, 15, 17, 19, 21, 23, 25, 27, 29, 31, 33, 35, 37, or 39, or a complement thereof, under stringent conditions. Such variants generally will retain the claimed activity. Variants include polypeptides that differ in amino acid sequence due to mutagenesis. Variant proteins encompassed by the present invention are insecticidally active.

Variant proteins can also be designed that differ at the primary amino acid sequence level and which retain the same or similar overall essential three-dimensional structure, surface charge distribution, and the like. See e.g. U.S. Pat. No. 7,058,515; Larson et al., (2002); Crameri et al., (1997); Stemmer, W. P. C. (1994a); Stemmer, W. P. C. (1994b) “Stemmer, W. P. C. (1995); Crameri et al., (1996a); and Crameri et al., (1996b).

Certain toxins of the subject invention have been specifically exemplified herein. Since these toxins are merely exemplary of the toxins of the subject invention, it should be readily apparent that the subject invention comprises variant or equivalent toxins (and nucleotide sequences coding for equivalent toxins) having the same or similar pesticidal activity of the exemplified toxin. Equivalent toxins will have amino acid homology with an exemplified toxin. The amino acid identity will typically be greater than 60%, preferably be greater than 75%, more preferably greater than 80%, more preferably greater than 90%, and can be greater than 95%. The amino acid homology will be highest in critical regions of the toxin which account for biological activity or are involved in the determination of three-dimensional configuration which ultimately is responsible for the biological activity. In this regard, certain amino acid substitutions are acceptable and can be expected if these substitutions are in regions which are not critical to activity or are conservative amino acid substitutions which do not affect the three-dimensional configuration of the molecule. For example, amino acids may be placed in the following classes: non-polar, uncharged polar, basic, and acidic. Conservative substitutions whereby an amino acid of one class is replaced with another amino acid of the same type fall within the scope of the subject invention so long as the substitution does not materially alter the biological activity of the compound. Table 1 provides a listing of examples of amino acids belonging to each class.

TABLE 1
Classes of Amino Acids and Examples.

| Class of Amino Acid | Examples of Amino Acids |
|---|---|
| Nonpolar Side Chains | Ala, Val, Leu, Ile, Pro, Met, Phe, Trp |
| Uncharged Polar Side Chains | Gly, Ser, Thr, Cys, Tyr, Asn, Gln |
| Acidic Side Chains | Asp, Glu |
| Basic Side Chains | Lys, Arg, His |
| Beta-branched Side Chains | Thr, Val, Ile |
| Aromatic Side Chains | Tyr, Phe, Trp, His |

In some instances, non-conservative substitutions can also be made. The critical factor is that these substitutions must not significantly detract from the biological activity of the toxin.

Preferred insecticidal toxins proteins of the present invention are encoded by a nucleotide sequence sufficiently identical to the nucleotide sequences of SEQ ID NOs:1, 3, 5, 7, 9, 11, 13, 15, 17, 19, 21, 23, 25, 27, 29, 31, 33, 35, 37 or 39. By “sufficiently identical” is intended an amino acid or nucleotide sequence that has at least about 60% or 65% sequence identity, preferably about 70% or 75% sequence identity, more preferably about 80% or 85% sequence identity, most preferably about 90%, 91%, 92%, 93%, 94%, 95%, 96%, 97%, 98% or 99% sequence identity compared to a reference sequence as analyzed by one of the alignment programs described herein, employing standard parameters. One of skill in the art will recognize that these values can be appropriately adjusted to determine corresponding identity of proteins encoded by two nucleotide sequences by taking into account codon degeneracy, amino acid similarity, reading frame positioning, and the like.

To determine the percent identity of two amino acid sequences or of two nucleic acid sequences, the sequences are aligned for optimal comparison purposes. The percent identity between the two sequences is a function of the number of identical positions shared by the sequences (i.e. percent identity=number of identical positions/total number of positions (e.g. overlapping positions)×100). In one embodiment, the two sequences are the same length. The percent identity between two sequences can be determined using techniques similar to those described below, with or without allowing gaps. In calculating percent identity, typically exact matches are counted.

The determination of percent identity between two sequences can be accomplished using a mathematical algorithm. A nonlimiting example of a mathematical algorithm utilized for the comparison of two sequences is the algorithm of Karlin and Altschul (1990), modified as in Karlin and Altschul (1993). Such an algorithm is incorporated into the BLASTN and BLASTX programs of Altschul et al., (1990). BLAST searches may be conveniently used to identify sequences homologous (similar) to a query sequence in nucleic or protein databases. BLAST nucleotide searches can be performed with the BLASTN program, score=100, word length=12, to identify nucleotide sequences having homology to claimed nucleic acid molecules of the invention. BLAST protein searches can be performed with the BLASTX program, score=50, word length=3, to identify amino acid sequences having homology to claimed insecticidal protein molecules of the invention.

To obtain gapped alignments for comparison purposes, Gapped BLAST can be utilized as described in Altschul et al., (1997). Alternatively, PSI-Blast can be used to perform an iterated search that detects distant relationships between molecules Altschul et al., (1997). When utilizing BLAST, Gapped BLAST, and PSI-Blast programs, the default parameters of the respective programs (e.g. BLASTX and BLASTN) can be used. See www.ncbi.nlm.nih.gov. Alignment may also be performed manually by inspection.

A non-limiting example of a mathematical algorithm utilized for the comparison of sequences is the ClustalW algorithm (Thompson et al., (1994). ClustalW compares sequences and aligns the entirety of the amino acid or DNA sequence, and thus can provide data about the sequence conservation of the entire amino acid sequence or nucleotide sequence. The ClustalW algorithm is used in several commercially available DNA/amino acid analysis software packages, such as the ALIGNX module of the Vector NTI Program Suite (Invitrogen, Inc., Carlsbad, CA). When aligning amino acid sequences with ALIGNX, one may conveniently use the default settings with a Gap open penalty of 10, a Gap extend penalty of 0.1 and the blosum63mt2 comparison matrix. After aligning two protein sequences with ALIGNX, the percent amino acid similarity (consensus) or identity between the two sequences can be assessed. When aligning two DNA sequences with ALIGNX, one may conveniently use the default settings with a Gap open penalty of 15, a Gap extend penalty of 6.6 and the swgapdnamt comparison matrix. After aligning two DNA sequences with ALIGNX, the percent identity between the two sequences can be assessed.

A second non-limiting example of a software program useful for analysis of ClustalW alignments is GeneDoc™ (developed by Karl Nicholas, http://iubio.bio.indiana.edu/soft/molbio/ibmpc/genedoc-readme.html). GeneDoc™ allows assessment of amino acid (or DNA) similarity and identity between multiple proteins.

Another non-limiting example of a mathematical algorithm utilized for the comparison of sequences is the algorithm of Myers and Miller (1988). Such an algorithm is incorporated into the wSTRETCHER program, which is part of the wEMBOSS sequence alignment software package (available at http://emboss.sourceforge.net/). STRETCHER calculates an optimal global alignment of two sequences using a modification of the classic dynamic programming algorithm which uses linear space. The output is a standard alignment file. The substitution matrix, gap insertion penalty and gap extension penalties used to calculate the alignment may be specified. When utilizing the STRETCHER program for comparing nucleotide sequences, a Gap open penalty of 16 and a Gap extend penalty of 4 can be used. The scoring matrix file for comparing DNA sequences is EDNAFULL. When used for comparing amino acid sequences, a Gap open penalty of 12 and a Gap extend penalty of 2 can be used. The scoring matrix file for comparing protein sequences is EBLOSUM62.

A further non-limiting example of a mathematical algorithm utilized for the comparison of sequences is the algorithm of Needleman and Wunsch (1970), which is incorporated in the sequence alignment software packages GAP Version 10 and wNEEDLE (http://emboss.sourceforge.net/). GAP version 10 may be used to determine sequence identity or similarity using the following parameters: for a nucleotide sequence, % identity and % similarity are found using GAP Weight of 50 and Length Weight of 3, and the nwsgapdna. cmp scoring matrix. For amino acid sequence comparison, % identity or % similarity fare determined using GAP weight of 8 and length weight of 2, and the BLOSUM62 scoring program. wNEEDLE reads two input sequences, finds the optimum alignment (including gaps) along their entire length, and writes their optimal global sequence alignment to file. The algorithm uses a dynamic programming method to ensure the alignment is optimum, by exploring all possible alignments and choosing the best. A scoring matrix is read that contains values for every possible residue or nucleotide match. wNEEDLE finds the alignment with the maximum possible score where the score of an alignment is equal to the sum of the matches taken from the scoring matrix, minus penalties arising from opening and extending gaps in the aligned sequences. The substitution matrix and gap opening and extension penalties are user-specified. When amino acid sequences are compared, a default Gap open penalty of 10, a Gap extend penalty of 0.5, and the EBLOSUM62 comparison matrix are used. When DNA sequences are compared using wNEEDLE, a Gap open penalty of 10, a Gap extend penalty of 0.5, and the EDNAFULL comparison matrix are used.

Equivalent programs may also be used. By “equivalent program” is intended any sequence comparison program that, for any two sequences in question, generates an alignment having identical nucleotide residue matches and an identical percent sequence identity when compared to the corresponding alignment generated by ALIGNX, wNEEDLE, or wSTRETCHER. The % identity is the percentage of identical matches between the two sequences over the reported aligned region (including any gaps in the length) and the % similarity is the percentage of matches between the two sequences over the reported aligned region (including any gaps in the length).

Toxin fragments and equivalents. Fragments and equivalents which retain the pesticidal activity of the exemplified toxins would be within the scope of the subject invention. Also, because of the redundancy of the genetic code, a variety of different DNA sequences can encode the amino acid sequences disclosed herein. It is well within the skill of a person trained in the art to create these alternative DNA sequences encoding the same, or essentially the same, toxins. These variant DNA sequences are within the scope of the subject invention. As used herein, reference to “essentially the same” sequence refers to sequences which have amino acid substitutions, deletions, additions, or insertions which do not materially affect pesticidal activity. Fragments retaining pesticidal activity are also included in this definition.

Alterations can be made at the amino or carboxy terminus of the insecticidal proteins and variants of the invention that result in polypeptides that retain biological activity. Fragments or biologically active portions include polypeptide fragments comprising amino acid sequences sufficiently identical to the amino acid sequence set forth in SEQ ID NOs:2, 4, 6, 8, 10, 12, 14, 16, 18, 20, 22, 24, 26, 28, 30, 32, 34, 36, 38, and 40. A biologically active portion of a delta endotoxin protein can be a polypeptide that is, for example, 10, 25, 50, 100, or more amino acids in length. Such biologically active portions can be prepared by recombinant protein engineering techniques well known in the art and evaluated for insecticidal activity. Methods for measuring pesticidal activity are well known in the art. As used herein a fragment encompasses at least 8 contiguous amino acids of SEQ ID NOs:2, 4, 6, 8, 10, 12, 14, 16, 18, 20, 22, 24, 26, 28, 30, 32, 34, 36, 38, and 40. The invention encompasses other fragments, however, such as any fragment in the protein greater than about 10, 20, 30, 50, 100, 150, 200, 250, 300, 350, 400, 450, 500, 550, 600, 650, 700, 750, 800, 850, 900, 950, 1000, 1050, 1100, 1150, or 1200 amino acids, up to the full length of the insecticidal proteins or variant proteins of SEQ ID NOs:2, 4, 6, 8, 10, 12, 14, 16, 18, 20, 22, 24, 26, 28, 30, 32, 34, 36, 38, and 40.

Fragments with improved biological activity, pest spectrum, or the ability to control resistant insect populations are also provided in the present invention. Modifications can be made to Cry proteins to produce fragments with improved pore formation and thereby pesticidal activity. In the case of three-domain Cry proteins, domain 1 is comprised of seven α-helices involved in pore formation in the midgut of susceptible insects. Modified DIG proteins with improved activity can be designed to have N-terminal deletions in regions with putative secondary structure homology to α-helix 1 and α-helix 2 of domain 1.

Proteases may be used to directly obtain active fragments of these toxins. A fragment of a claimed insecticidal toxin will comprise at least about 15, 25, 30, 50, 75, 100, 125, 150, 175, 200, 250, 300, 350, 400, 450, 500, 550, 600, 650, 700, 800, 900, 1000, 1100 or 1200 contiguous amino acids, or up to the total number of amino acids present in a full-length insecticidal toxin of the invention (for example, 625 amino acids for SEQ ID NO:2, or 625 amino acids for SEQ ID NO:4).

Core toxin and Protoxin chimeras. A majority of Bacillus thuringiensis delta-endotoxin crystal protein molecules are composed of two functional segments. The protease-resistant core toxin is the first segment and corresponds to about the first half of the protein molecule. The approximately C-terminal half of the molecule is the second segment. For purposes of this application, this second segment will be referred to herein as the “protoxin segment.” The protoxin segment is believed to participate in toxin crystal formation (Arvidson et al., (1989)). The full 130 kDa toxin molecule is rapidly processed to the resistant core segment by protease in the insect gut. The protoxin segment may thus convey a partial insect specificity for the toxin by limiting the accessibility of the core to the insect by reducing the protease processing of the toxin molecule (Haider et al., (1986)) or by reducing toxin solubility (Aronson et al., (1991)).

Chimeric proteins advantageously joined within the toxin domains of Cry1Fa and Cry1Ab have been reported (U.S. Pat. No. 5,527,883). Other success in the area has been reported in the literature. For example, the construction of hybrid delta-endotoxins is reported in the following related art. Intl. Pat. Appl. Publ. No. WO 95/30753 discloses the construction of hybrid B. thuringiensis delta-endotoxins for production in Pseudomonas fluorescens in which the non-toxic protoxin fragment of Cry1F has been replaced by the non-toxic protoxin fragment from the Cry1Ac/Cry1Ab that is disclosed in U.S. Pat. No. 5,128,130. That patent also discloses the construction of hybrid B. thuringiensis delta-endotoxins for production in P. fluorescens in which a portion of the non-toxic protoxin segment of Cry1Ac is replaced with the corresponding non-toxic protoxin fragment of Cry1Ab. U.S. Pat. No. 5,055,294 discloses the construction of a specific hybrid delta-endotoxin between Cry1Ac (amino acid residues 1-466) and Cry1Ab (amino acid residues 466-1155) for production in P. fluorescens. Although the aforementioned patent discloses the construction of a hybrid toxin within the active toxin segment, no specifics are presented in regard to the hybrid toxin's insecticidal activity. International Patent Application Publication No. WO 95/30752 discloses the construction of hybrid B. thuringiensis delta-endotoxins for production in P. fluorescens in which the non-toxic protoxin segment of Cry1C is replaced by the non-toxic protoxin segment from Cry1Ab. The afore-mentioned application further discloses that the activity against Spodoptera exigua for the hybrid delta-endotoxin is improved over that of the parent active toxin, Cry1C. International Patent Application Publication No. WO 95/06730 discloses the construction of a hybrid B. thuringiensis delta-endotoxin consisting of domains 1 and 2 of Cry1E coupled to domain 3 and the non-toxic protoxin segment of Cry1C. Insect bioassays performed against Manduca sexta (sensitive to Cry1C and Cry1E), Spodoptera exigua (sensitive to Cry1C), and Mamestra brassicae (sensitive to Cry1C) show that the hybrid Cry1E/Cry1C hybrid toxin is active against M. sexta, S. exigua, and M. brassicae. The bioassay results were expressed as EC₅₀ values (toxin concentration giving a 50% growth reduction) rather than LC₅₀ values (toxin concentration giving 50% mortality). Although the delta-endotoxins used for bioassay were produced in B. thuringiensis, only artificially-generated active segments of the delta-endotoxins were used, not the naturally-produced crystals typically produced by B. thuringiensis that are present in commercial B. thuringiensis formulations. Bioassay results indicated that the LC₅₀ values for the hybrid Cry1E/Cry1C crystal against S. frugiperda were 1.5 to 1.7 fold lower (i.e. were more active) than for native Cry1C. This art also discloses the construction of a hybrid B. thuringiensis delta-endotoxin between Cry1Ab (domains 1 and 2) and Cry1C (domain 3 and the non-toxic protoxin segment), although no data are given regarding the hybrid toxin's activity or usefulness.

Lee et al., (1995) report the construction of hybrid B. thuringiensis delta-endotoxins between Cry1Ac and Cry1Aa within the active toxin segment. Artificially generated active segments of the hybrid toxins were used to examine protein interactions in susceptible insect brush border membranes vesicles (BBMV). The bioactivity of the hybrid toxins was not reported. Honee et al., (1991) report the construction of hybrid delta-endotoxins between Cry1C (domain 1) and Cry1Ab (domains 2 and 3) and the reciprocal hybrid between Cry1Ab (domain 1) and Cry1C (domains 2 and 3). These hybrids failed to show any significant increase in activity against susceptible insects. Furthermore, the Cry1C (domain 1)/Cry1Ab (domains 2 and 3) hybrid toxin was found to be hypersensitive to protease degradation. A report by Schnepf et al., (1990) discloses the construction of Cry1Ac hybrid toxin in which a small portion of domain 2 was replaced by the corresponding region of Cry1Aa, although no significant increase in activity against susceptible insect larvae was observed.

The chimeric toxins of the subject invention comprise a full core N-terminal toxin portion of a B.t. toxin and, at some point past the end of the toxin portion, the protein has a transition to a heterologous protoxin sequence. The transition to the heterologous protoxin segment can occur at approximately the native toxin/protoxin junction or a portion of the native protoxin (extending past the toxin portion) can be retained with the transition to the heterologous protoxin occurring downstream. For example, a chimeric toxin of the subject invention may have the full toxin portion of a modified Cry1Ca toxin such as amino acids 1-628 of DIG-473 or DIG-465 and a heterologous protoxin segment (amino acids 629 to the C-terminus). In a preferred embodiment, the heterologous protoxin segment portion is taken from Cry1Ab.

A person skilled in this art will appreciate that B.t. toxins, even within a certain class, will vary to some extent in length and in the precise location of the transition from the core toxin portion to protoxin portion. The transition from core toxin portion to protoxin portion will typically occur at between about 50% to about 60% of the full length toxin. The chimeric toxin of the subject invention includes the full expanse of this core N-terminal toxin portion, such is the full 628 amino acid length of IRDIG544.12 insecticidal toxin protein. SEQ ID NO:15 discloses the 1887 nucleotide sequence of the DIG-465-encoding DNA, of which the 5′-terminal 1887 nucleotides comprise the coding region for the core toxin segment of Cry1Ca with a mutation L57A (leucine at amino acid position 57 substituted for alanine), one embodiment of the subject invention. SEQ ID NO:16 discloses the 628 amino acid sequence of the full-length DIG-465 polypeptide, of which the N-terminal core portion of Cry1Ca with the above mentioned amino acid substitutions. SEQ ID NO:23 discloses the 1887 nucleotide sequence of DIG-473-encoding DNA, which comprises the coding region for the core toxin segment of Cry1Ca with a mutation F596M (phenylalanine at amino acid position 596, substituted for methionine), another subject of the invention. SEQ ID NO:24 discloses the 628 amino acid sequence of the full-length DIG-473 polypeptide, which comprises the portion of Cry1Ca with the above mentioned amino acid substitutions.

With regard to the protoxin portion, the full expanse of the native Cry1Ab protoxin portion extends from the end of the toxin portion of the Cry1Ab full length protein to the C-terminus of the molecule. Attention is drawn to the last about 100 to 150 amino acids of this protoxin, which are most critical to include in the chimeric toxin of the subject invention.

Because Cry proteins have selective insecticidal activity, most are active on a limited range of target pests. There is, therefore, a need to further improve the biological activity attributes of Cry proteins. Cry proteins with unique binding characteristics and modes of action are useful in strategies to expand the range of insect pests controlled or counter the development of B.t. resistance.

Domain III modifications. As described herein, the B.t. insecticidal toxins of the subject invention are 3-domain type toxins, comprising Domain I, Domain II, and Domain III. Domain III binds certain classes of receptor proteins and perhaps participates in insertion of an oligomeric toxin pre-pore. Specific hybrid toxins that comprised domain III substitutions were shown to have superior toxicity against Spodoptera exigua (de Maagd et al., 1996) and guidance exists on the design of the Cry toxin domain swaps (Knight et al., 2004).

Domain I modifications. Numerous studies using biochemical and molecular approaches have provided information on the determinants of Cry protein binding and insertion into insect midgut membranes (reviewed in Piggot and Ellar, 2007). Domain I from Cry1A and Cry3A proteins has been studied for the ability to insert and form pores in membranes. α-helices 4 and 5 of domain I play key roles in membrane insertion and pore formation (Walters et al., 1993, Gazit et al., 1998; Nunez-Valdez et al., 2001), with the other helices proposed to contact the membrane surface like the ribs of an umbrella (Gazit et al., 1998).

Alpha-helix 3 appears in some instances to be required for oligomeric pre-pore formation and toxicity. Some α-helix 3 mutants are able to bind receptors but do not form oligomers and are non-toxic to Manduca sexta (reviewed in Jimenez-Juarez et al., 2008). However, proteolytically activated forms of Cry3Aa1 lack α-helices 1, 2 and 3 (Carroll et al., 1997).

Alpha-helix 1 is removed following receptor binding. Gomez et al., (2002) found that Cry1Ab oligomers formed upon BBMV receptor binding lacked the α-helix 1 portion of domain I. Also, Soberon et al., (2007) have shown that N-terminal deletion mutants of Cry1Ab and Cry1Ac which lack approximately 60 amino acids encompassing α-helix 1 on the three dimensional Cry structure are capable of assembling monomers of molecular weight about 60 kDa into pre-pores in the absence of cadherin binding. These N-terminal deletion mutants were reported to be active on Cry-resistant insect larvae. Furthermore, Diaz-Mendoza et al., (2007) described Cry1Ab fragments of 43 kDa and 46 kDa that retained activity on Mediterranean corn borer (Sesamia nonagrioides). These fragments were demonstrated to include amino acid residues 116 to 423; however the precise amino acid sequences were not elucidated and the mechanism of activity of these proteolytic fragments is unknown. The results of Gomez et al., (2002), Soberon et al., 2007 and Diaz-Mendoza et al., (2007) contrast with those of Hofte et al., (1986), who reported that deletion of 36 amino acids from the N-terminus of Cry1Ab resulted in loss of insecticidal activity.

Anti-toxin antibodies. Equivalent toxins and/or genes encoding these equivalent toxins can be derived from Bacillus thuringiensis isolates and/or DNA libraries using the teachings provided herein. There are a number of methods for obtaining the pesticidal toxins of the instant invention. For example, antibodies immunoreactive to the pesticidal toxins disclosed and claimed herein can be used to identify and isolate other toxins from a mixture of proteins. Specifically, antibodies may be raised to the portions of the toxins which are most constant and most distinct from other B.t. toxins. These antibodies can then be used to specifically identify equivalent toxins with the characteristic activity by, for example, immunoprecipitation, enzyme linked immunosorbent assay (ELISA), or immunoblotting (western blotting). Antibodies to the toxins disclosed herein, or to equivalent toxins, or fragments of these toxins, can readily be prepared using standard procedures in this art. The genes which encode these toxins can then be obtained from the microorganisms that produce the toxins.

Once the B.t. insecticidal toxin has been isolated, antibodies specific for the toxin may be raised by conventional methods that are well known in the art. Repeated injections into a host of choice over a period of weeks or months will elicit an immune response and result in significant anti-B.t. toxin serum titers. Preferred hosts are mammalian species and more highly preferred species are rabbits, goats, sheep, and mice. Blood drawn from such immunized animals may be processed by established methods to obtain antiserum (polyclonal antibodies) reactive with the B.t. insecticidal toxin. The antiserum may then be affinity purified by adsorption to the toxin according to techniques known in the art. Affinity purified antiserum may be further purified by isolating the immunoglobulin fraction within the antiserum using procedures known in the art. The resulting material will be a heterogeneous population of immunoglobulins reactive with the B.t. insecticidal toxin.

Anti-B.t. toxin antibodies may also be generated by preparing a semi-synthetic immunogen consisting of a synthetic peptide fragment of the B.t. insecticidal toxin conjugated to an immunogenic carrier. Numerous schemes and instruments useful for making peptide fragments are well known in the art. Many suitable immunogenic carriers such as bovine serum albumin (BSA) or keyhole limpet hemocyanin are also well known in the art, as are techniques for coupling the immunogen and carrier proteins. Once the semi-synthetic immunogen has been constructed, the procedure for making antibodies specific for the B.t. insecticidal toxin fragment is identical to those used for making antibodies reactive with natural B.t. toxin.

Anti-B.t. toxin monoclonal antibodies (MAbs) are readily prepared using purified B.t. insecticidal toxin. Methods for producing MAbs have been practiced for over 15 years and are well known to those of ordinary skill in the art. Repeated intraperitoneal or subcutaneous injections of purified B.t. insecticidal toxin in adjuvant will elicit an immune response in most animals. Hyperimmunized B-lymphocytes are removed from the animal and fused with a suitable fusion partner cell line capable of being cultured indefinitely. Preferred animals whose B-lymphocytes may be hyperimmunized and used in the production of MAbs are mammals. More preferred animals are rats and mice and most preferred is the BALB/c mouse strain.

Numerous mammalian cell lines are suitable fusion partners for the production of hybridomas. Many such lines are available from the American Type Culture Collection (ATCC, Manassas, VA) and commercial suppliers. Preferred fusion partner cell lines are derived from mouse myelomas and the HL-1® Friendly myeloma-653 cell line (Ventrex, Portland, ME) is most preferred. Once fused, the resulting hybridomas are cultured in a selective growth medium for one to two weeks. Two well known selection systems are available for eliminating unfused myeloma cells, or fusions between myeloma cells, from the mixed hybridoma culture. The choice of selection system depends on the strain of mouse immunized and myeloma fusion partner used. The AAT selection system, described by Taggart and Samloff (1983), may be used; however, the HAT (hypoxanthine, aminopterin, thymidine) selection system, described by Littlefield (1964), is preferred because of its compatibility with the preferred mouse strain and fusion partner mentioned above. Spent growth medium is then screened for immunospecific MAb secretion. Enzyme linked immunosorbent assay (ELISA) procedures are best suited for this purpose; though, radioimmunoassays adapted for large volume screening are also acceptable. Multiple screens designed to consecutively pare down the considerable number of irrelevant or less desired cultures may be performed. Cultures that secrete MAbs reactive with the B.t. insecticidal toxin may be screened for cross-reactivity with known B.t. insecticidal toxins. MAbs that preferentially bind to the preferred B.t. insecticidal toxin may be isotyped using commercially available assays. Preferred MAbs are of the IgG class, and more highly preferred MAbs are of the IgG1 and IgG2a subisotypes.

Hybridoma cultures that secrete the preferred MAbs may be sub-cloned several times to establish monoclonality and stability. Well known methods for sub-cloning eukaryotic, non-adherent cell cultures include limiting dilution, soft agarose and fluorescence activated cell sorting techniques. After each subcloning, the resultant cultures preferably are re-assayed for antibody secretion and isotype to ensure that a stable preferred MAb-secreting culture has been established.

The anti-B.t. toxin antibodies are useful in various methods of detecting the claimed B.t. insecticidal toxin of the instant invention, and variants or fragments thereof. It is well known that antibodies labeled with a reporting group can be used to identify the presence of antigens in a variety of milieus. Antibodies labeled with radioisotopes have been used for decades in radioimmunoassays to identify, with great precision and sensitivity, the presence of antigens in a variety of biological fluids. More recently, enzyme labeled antibodies have been used as a substitute for radiolabeled antibodies in the ELISA assay. Further, antibodies immunoreactive to the B.t. insecticidal toxin of the present invention can be bound to an immobilizing substance such as a polystyrene well or particle and used in immunoassays to determine whether the B.t. toxin is present in a test sample.

In one preferred embodiment, insecticidal proteins or a variant is delivered orally through a transgenic plant comprising a nucleic acid sequence that expresses a toxin of the present invention. The present invention provides a method of producing an insect-resistant transgenic plant, comprising introducing a nucleic acid molecule of the invention into the plant wherein the toxin is expressible in the transgenic plant in an effective amount to control an insect. In a non-limiting example, a basic cloning strategy may be to subclone full length or modified Cry coding sequences (CDS) into a plant expression plasmid at NcoI and SacI restriction sites. The resulting plant expression cassettes containing the appropriate Cry coding region under the control of plant expression elements, (e.g., plant expressible promoters, 3′ terminal transcription termination and polyadenylate addition determinants, and the like) are subcloned into a binary vector plasmid, utilizing, for example, Gateway® technology or standard restriction enzyme fragment cloning procedures. LR Clonase™ (Invitrogen) for example, may be used to recombine the full length and modified gene plant expression cassettes into a binary plant transformation plasmid if the Gateway® technology is utilized. It is convenient to employ a binary plant transformation vector that harbors a bacterial gene that confers resistance to the antibiotic spectinomycin when the plasmid is present in E. coli and Agrobacterium cells. It is also convenient to employ a binary vector plasmid that contains a plant-expressible selectable marker gene that is functional in the desired host plants. Examples of plant-expressible selectable marker genes include but are not limited to aminoglycoside phosphotransferase gene of transposon Tn5 (Aph II) which encodes resistance to the antibiotics kanamycin, neomycin and G418, as well as those genes which code for tolerance to glyphosate; hygromycin; methotrexate; phosphinothricin (bialaphos), imidazolinones, sulfonylureas and triazolopyrimidine herbicides, such as chlorosulfuron, bromoxynil, dalapon and the like.

Alternatively, the plasmid structure of the binary plant transformation vector containing the DIG-465, DIG-473, DIG-468, DIG-483, DIG-462, DIG-463, DIG-464, DIG-466, DIG-467, DIG-469, DIG-474, DIG-482, DIG-485, DIG-487, IRDIG544.8, IRDIG544.9, IRDIG544.11, or IRDIG544.12 gene insert is performed by restriction digest fingerprint mapping of plasmid DNA prepared from candidate Agrobacterium isolates by standard molecular biology methods well known to those skilled in the art of Agrobacterium manipulation.

Those skilled in the art of obtaining transformed plants via Agrobacterium-mediated transformation methods will understand that other Agrobacterium strains besides Z707S may be used, and the choice of strain may depend upon the identity of the host plant species to be transformed.

Following are examples which illustrate procedures for practicing the invention. These examples should not be construed as limiting. All percentages are by weight and all solvent mixture proportions are by volume unless otherwise noted. All patents, patent applications, provisional applications, and publications referred to or cited herein are incorporated by reference in their entirety to the extent they are not inconsistent with the explicit teachings of this specification. Unless specifically indicated or implied, the terms “a”, “an”, and “the” signify “at least one” as used herein.

### Example 1

Design of a Plant-Optimized Version of the Coding Sequence for B.t. Insecticidal Proteins.

A DNA sequence having a plant codon bias was designed and synthesized to produce the insecticidal proteins in transgenic monocot and dicot plants. A codon usage table for maize (Zea mays L.) was calculated from 706 protein coding sequences (CDS) obtained from sequences deposited in GenBank. Codon usage tables for tobacco (Nicotiana tabacum, 1268 CDS), canola (Brassica napus, 530 CDS), cotton (Gossypium hirsutum, 197 CDS), and soybean (Glycine max; ca. 1000 CDS) were downloaded from data at the website http://www.kazusa.or.jp/codon/. A biased codon set that comprises highly used codons common to both maize and dicot datasets, in appropriate weighted average relative amounts, was calculated after omitting any redundant codon used less than about 10% of total codon uses for that amino acid in either plant type. To derive a plant optimized sequence encoding the insecticidal protein, codon substitutions to the insecticidal protein DNA sequences were made such that the resulting DNA sequence had the overall codon composition of the plant-optimized codon bias table. Further refinements of the sequence were made to eliminate undesirable restriction enzyme recognition sites, potential plant intron splice sites, long runs of A/T or C/G residues, and other motifs that might interfere with RNA stability, transcription, or translation of the coding region in plant cells. Other changes were made to introduce desired restriction enzyme recognition sites, and to eliminate long internal Open Reading Frames (frames other than +1). These changes were all made within the constraints of retaining the plant-biased codon composition. To complete the design, a sequence encoding translational Stop codons in all 6 open reading frames was added to the 3′ end of the coding regions, and appropriate restriction recognition sites were added to the 5′ and 3′ ends of the sequences. Synthesis of the designed sequence was performed by a commercial vendor (DNA2.0, Menlo Park, CA). Additional guidance regarding the production of synthetic genes can be found in, for example, WO 97/13402 and U.S. Pat. No. 5,380,831.

Plant-optimized DNA sequences encoding DIG proteins of the subject invention (SEQ ID NOs:2, 4, 6, 8, 10, 12, 14, 16, 18, 20, 22, 24, 26, 28, 30, 32, 34, 36, 38, and 40) are disclosed as SEQ ID NOs:1, 3, 5, 7, 9, 11, 13, 15, 17, 19, 21, 23, 25, 27, 29, 31, 33, 35, 37, and 39. DNA molecules comprising sequences disclosed in SEQ ID NOs: 1, 3, 5, 7, 9, 11, 13, 15, 17, 19, 21, 23, 25, 27, 29, 31, 33, 35, 37, and 39 were synthetically assembled by a commercial entity (DNA2.0).

### Example 2

### Construction of Expression Plasmids Encoding Insecticidal Toxins and Expression in Bacterial Hosts.

Standard cloning methods were used in the construction of Pseudomonas fluorescens (Pf) expression plasmids engineered to produce DIG-465 (SEQ ID NO:16), DIG-473 (SEQ ID NO:24), DIG-468 (SEQ ID NO:2), DIG-483 (SEQ ID NO:4), DIG-462 (SEQ ID NO:10), DIG-463 (SEQ ID NO:12), DIG-464 (SEQ ID NO:14), DIG-466 (SEQ ID NO:18), DIG-467 (SEQ ID NO:20), DIG-469 (SEQ ID NO:22), DIG-474 (SEQ ID NO:26), DIG-482 (SEQ ID NO:28), DIG-485 (SEQ ID NO:6), and DIG-487 (SEQ ID NO:8) proteins encoded by plant-optimized coding regions. Restriction endonucleases were obtained from New England BioLabs (NEB; Ipswich, MA) and T4 DNA Ligase (Invitrogen Corporation, Carlsbad, CA) was used for DNA ligation.

The basic cloning strategy entailed subcloning the DIG-465, DIG-473, DIG-468, DIG-483, DIG-462, DIG-463, DIG-464, DIG-466, DIG-467, DIG-469, DIG-474, DIG-482, DIG-485, or DIG-487 toxin coding sequence (CDS) into pDOW1169 at restriction sites such as SpeI and XhoI, whereby it was placed under the expression control of the Ptac promoter and the rrnBT1T2 terminator from plasmid pKK223-3 (PL Pharmacia, Milwaukee, WI). pDOW1169 is a low copy plasmid with the RSF1010 origin of replication, a pyrF gene, and a ribosome binding site preceding the restriction enzyme recognition sites into which DNA fragments containing protein coding regions may be introduced, (US Patent Application US20080193974). The expression plasmid was transformed by electroporation into DC454 (a near wild-type P. fluorescens strain having mutations ΔpyrF and lsc::lacIQI), or its derivatives, recovered in SOC-Soy hydrolysate medium, and plated on selective medium (M9 glucose agar lacking uracil, Sambrook et al., supra). Details of the microbiological manipulations are available in Squires, C. H. et al., (2004), US Patent Application 20060008877, US Patent Application 20080193974, and US Patent Application 20080058262, incorporated herein by reference. Strains were validated by restriction digestion of miniprep plasmid DNA.

Growth and Expression Analysis in Shake Flasks. Production of DIG-465, DIG-473, DIG-468, DIG-483, DIG-462, DIG-463, DIG-464, DIG-466, DIG-467, DIG-469, DIG-474, DIG-482, DIG-485, or DIG-487 toxin for characterization and insect bioassay was accomplished by shake-flask-grown P. fluorescens strains harboring expression constructs. Seed cultures grown in M9 medium supplemented with 1% glucose and trace elements were used to inoculate 50 mL of defined minimal medium with 5% glycerol (Teknova Cat. #3D7426, Hollister, CA). Expression of the insecticidal protein toxin gene via the Ptac promoter was induced by addition of isopropyl-β-D-1-thiogalactopyranoside (IPTG) after an initial incubation of 24 hours at 30° C. with shaking. Cultures were sampled at the time of induction and at various times post-induction. Cell density was measured by optical density at 600 nm (OD₆₀₀). Other culture media suitable for growth of Pseudomonas fluorescens may also be utilized, for example, as described in Huang et al., 2007 and US Patent Application 20060008877.

Cell Fractionation and SDS-PAGE Analysis of Shake Flask Samples. At each sampling time, 0.5 mL aliquots were centrifuged at 14000×g for five minutes. The cell pellets were frozen at −80° C. Soluble and insoluble fractions from frozen shake flask cell pellet samples were generated using BugBuster Master Mix (EMDMillipore® Darmstadt, Germany). Each cell pellet was resuspended in 0.5 mL BugBuster Master Mix™ solution and incubated with shaking at room temperature for 30 minutes. The samples were lysed using a beadbeater with 0.1 mm glass beads for 3 minutes. The lysate was centrifuged at 14,000 rpm for 5 minutes and the supernatant was recovered as the soluble fraction. The pellet (insoluble fraction) was then resuspended in an equal volume of extraction buffer (8 M urea, 0.5 M NaCl, 25 mM NaPO4, pH 10.4).

Samples were mixed 1:1 with 2× NuPAGE Tris Glycine SDS Sample Buffer (Invitrogen, Carlsbad, CA) containing dithiothreitol (DTT) and boiled for 5 minutes prior to loading onto Novex 4-20% Tris Glycine SDS polyacrylamide gel (Invitrogen, Carlsbad, CA). Electrophoresis was performed in the recommended Tris-Glycine buffer. Gels were stained with Bio-Safe Coomassie Stain according to the manufacturer's (Bio-Rad Inc., Hercules, CA) protocol and imaged using the GE Typhooon Series Imaging system (Pittsburgh, PA).

Inclusion body preparation. Cry protein inclusion body (IB) preparations were performed on cells from P. fluorescens fermentations that produced insoluble B.t. insecticidal protein, as demonstrated by SDS-PAGE and MALDI-MS (Matrix Assisted Laser Desorption/Ionization Mass Spectrometry). P. fluorescens fermentation pellets were thawed in a 37° C. water bath. The cells were resuspended to 25% w/v in lysis buffer (50 mM Tris, pH 7.5, 200 mM NaCl, 20 mM EDTA disodium salt (Ethylenediaminetetraacetic acid), 1% Triton X-100, and 5 mM Dithiothreitol (DTT); 5 mL/L of bacterial protease inhibitor cocktail (P8465 Sigma-Aldrich, St. Louis., MO) were added just prior to use). The cells were suspended using a hand-held homogenizer at the lowest setting (Tissue Tearor, BioSpec Products, Inc Bartlesville, OK). Lysozyme (25 mg of Sigma-Aldrich L7651, from chicken egg white) was added to the cell suspension by mixing with a metal spatula, and the suspension was incubated at room temperature for one hour. The suspension was cooled on ice for 15 minutes, then sonicated using a Branson Sonifier 250 (two 1-minute sessions, at 50% duty cycle, 30% output). Cell lysis was checked by microscopy. An additional 25 mg of lysozyme was added if necessary, and the incubation and sonication were repeated. When cell lysis was confirmed via microscopy, the lysate was centrifuged at 11,500×g for 25 minutes (4° C.) to form the IB pellet, and the supernatant was discarded. The IB pellet was suspended with 100 mL lysis buffer, homogenized with the hand-held mixer and centrifuged as above. The IB pellet was repeatedly washed by suspension (in 50 mL lysis buffer), homogenization, sonication, and centrifugation until the supernatant became colorless and the IB pellet became firm and off-white in color. For the final wash, the IB pellet was suspended in sterile-filtered (0.22 μm) distilled water containing 2 mM EDTA, and centrifuged. The final pellet was suspended in sterile-filtered distilled water containing 2 mM EDTA, and stored in 1 mL aliquots at −80° C.

SDS-PAGE analysis and quantification of protein in IB preparations was done by thawing a 1 mL aliquot of IB pellet and diluting 1:20 with sterile-filtered distilled water. The diluted sample was then boiled with 4× reducing sample buffer [250 mM Tris, pH 6.8, 40% glycerol (v/v), 0.4% Bromophenol Blue (w/v), 8% SDS (w/v) and 8% β-Mercapto-ethanol (v/v)] and loaded onto a Novex® 4-20% Tris-Glycine, 12+2 well gel (Invitrogen) run with 1× Tris/Glycine/SDS buffer (BioRad). The gel was run for 60 min at 200 volts then stained with Coomassie Blue (50% G-250/50% R-250 in 45% methanol, 10% acetic acid), and destained with 7% acetic acid, 5% methanol in distilled water. Quantification of target bands was done by comparing densitometric values for the bands against Bovine Serum Albumin (BSA) samples run on the same gel to generate a standard curve.

Solubilization of Inclusion Bodies. Six mL of inclusion body suspension from Pf clone containing DIG-465, DIG-473, DIG-468, DIG-483, DIG-462, DIG-463, DIG-464, DIG-466, DIG-467, DIG-469, DIG-474, DIG-482, DIG-485, or DIG-487 protein were centrifuged on the highest setting of an Eppendorf model 5415C microfuge (approximately 14,000×g) to pellet the inclusions. The storage buffer supernatant was removed and replaced with 25 mL of 100 mM sodium carbonate buffer, pH 11, in a 50 mL conical tube. Inclusions were resuspended using a pipette and vortexed to mix thoroughly. The tube was placed on a gently rocking platform at 4° C. overnight to extract the target protein. The extract was centrifuged at 30,000×g for 30 min at 4° C., and the resulting supernatant was concentrated 5-fold using an Amicon Ultra-15 regenerated cellulose centrifugal filter device (30,000 Molecular Weight Cutoff; Millipore). The sample buffer was then changed to 10 mM CAPS [3-(cyclohexamino)1-propanesulfonic acid] pH 10, using disposable PD-10 columns (GE Healthcare, Piscataway, NJ).

Gel electrophoresis. The concentrated extract was prepared for electrophoresis by diluting 1:50 in NuPAGE® LDS sample buffer (Invitrogen) containing 5 mM dithiothreitol as a reducing agent and heated at 95° C. for 4 minutes. The sample was loaded in duplicate lanes of a 4-12% NuPAGE® gel alongside five BSA standards ranging from 0.2 to 2 μg/lane (for standard curve generation). Voltage was applied at 200V using MOPS SDS running buffer (Invitrogen) until the tracking dye reached the bottom of the gel. The gel was stained with 0.2% Coomassie Blue G-250 in 45% methanol, 10% acetic acid, and destained, first briefly with 45% methanol, 10% acetic acid, and then at length with 7% acetic acid, 5% methanol until the background cleared. Following destaining, the gel was scanned with a Biorad Fluor-S MultiImager. The instrument's Quantity One v.4.5.2 Software was used to obtain background-subtracted volumes of the stained protein bands and to generate the BSA standard curve that was used to calculate the concentration of DIG-465, DIG-473, DIG-468, DIG-483, DIG-462, DIG-463, DIG-464, DIG-466, DIG-467, DIG-469, DIG-474, DIG-482, DIG-485, or DIG-487 protein in the stock solution.

The level of expression of DIG-465, DIG-473, DIG-468, DIG-483, DIG-463, DIG-464, DIG-466, DIG-467, DIG-469, DIG-474, DIG-482, DIG-485, and DIG-487 was compared to the level of expression of truncated Cry1Ca (DIG-462) when expressed in Pseudomonas fluorescens bacterial cells. Truncated Cry1Ca (DIG-462) expressed at approximately 1 g/l, whereas DIG-473 expressed at approximately 0.5 g/l. DIG-465 expressed at approximately 5-fold higher than truncated Cry1Ca, at 4.9 g/l. These in vitro results show that the L57A mutation results in greater expression of truncated Cry1Ca protein.

### Example 3

Insecticidal Activity of DIG Proteins Produced in Pseudomonas fluorescens

B.t. insecticidal toxins DIG-462, DIG-463, DIG-464, DIG-465, DIG-466, DIG-467, DIG-468, DIG-469, DIG-470, DIG-473, and DIG-474 were demonstrated to be active on Lepidopteran species including diamondback moth (DBM; Plutella xylostella (Linnaeus)) and fall armyworm (FAW, Spodoptera frugiperda (Smith)).

Sample preparation and bioassays. Inclusion body preparations in 10 mM CAPS pH10 were diluted appropriately in 10 mM CAPS, pH 10, and all bioassays contained a control treatment consisting of this buffer, which served as a background check for mortality or growth inhibition.

Protein concentrations in bioassay buffer were estimated by gel electrophoresis using BSA to create a standard curve for gel densitometry, which was measured using a BioRad imaging system (Fluor-S MultiImager with Quantity One software version 4.5.2). Proteins in the gel matrix were stained with Coomassie Blue-based stain and destained before reading.

Purified proteins were tested for insecticidal activity in bioassays conducted with neonate Lepidopteran larvae on artificial insect diet. Larvae of DBM and FAW were hatched from eggs obtained from a colony maintained by a commercial insectary (Benzon Research Inc., Carlisle, PA). Larvae of rFAW were hatched from eggs harvested from a proprietary colony (Dow AgroSciences LLC, Indianapolis, IN).

These bioassays were conducted in 128-well plastic trays specifically designed for insect bioassays (C-D International, Pitman, NJ). Each well contained 1.0 mL of multi-species Lepidoptera diet (Southland Products, Lake Village, AR). A 40 μL aliquot of protein sample was delivered by pipette onto the 1.5 cm² diet surface of each well (26.7 μL/cm²). Cry protein concentrations were calculated as the amount (ng) of DIG protein per square centimeter (cm²) of surface area in the well. The treated trays were held in a fume hood until the liquid on the diet surface had evaporated or was absorbed into the diet.

Within a few hours of eclosion, individual larvae were picked up with a moistened camel hair brush and deposited on the treated diet, one larva per well. The infested wells were then sealed with adhesive sheets of clear plastic, vented to allow gas exchange (C-D International, Pitman, NJ). Bioassay trays were held under controlled environmental conditions (28° C., ˜60% Relative Humidity, 16:8 [Light:Dark]) for 5 days, after which the total number of insects exposed to each protein sample, the number of dead insects, and the weight of surviving insects were recorded. Percent mortality and percent growth inhibition were calculated for each treatment. Growth inhibition (GI) was calculated as follows:

$$GI = [ 1 - ( TWIT / TNIT ) / ( TWIBC / TNIBC ) ]$$

where TWIT is the Total Weight of Insects in the Treatment,
TNIT is the Total Number of Insects in the Treatment
TWIBC is the Total Weight of Insects in the Background Check (Buffer control), and
TNIBC is the Total Number of Insects in the Background Check (Buffer control).

In the DBM bioassay, 10 and 300 ng/cm² of DIG-462, DIG-463, DIG-464, DIG-465, DIG-466, DIG-467, DIG-468, DIG-469, DIG-470, DIG-471, DIG-472, DIG-473, and DIG-474 were tested against the insect sp. The FAW were tested with inclusion body preparation of DIG-462, DIG-465, DIG-473 at 1× and 5× dilution rate. Percent mortality and growth inhibition results were compared.

Mortality was 100% at 300 ng/cm² for DIG-462, DIG-463, DIG-464, DIG-465, DIG-466, DIG-468, DIG-469, DIG-473, and DIG-474 treatments (Table 2 and Table 3). Growth inhibition was 70-90% inhibition of growth at 10 ng/cm² and 100% inhibition at 300 ng/cm² for DIG-465 and DIG-473 treatments (Table 2).

TABLE 2
Results of bioassay tests of DIG-462, DIG-465 and DIG-473 proteins
on DBM, measuring both mortality and growth inhibition

| Protein | Mortality | Growth Inhibition |
|---|---|---|
| DIG-462 | +++ | ++++ |
| DIG-465 | ++ | ++++ |
| DIG-473 | +++ | ++++ |

For Mortality ++ = 0-20% at 10 ng/cm² and 100% at 300 ng/cm², +++ = 30-60% at 10 ng/cm² and 100% at 300 ng/cm².
For Growth Inhibition ++++ = 70-90% inhibition of growth at 10 ng/cm² and 100% inhibition at 300 ng/cm².

TABLE 3
Bioassay results of protein mutants tested against
DBM at 10 ng/cm² and 300 ng/cm² concentrations.

| | % Mortality | % Mortality |
|---|---|---|
| Protein | at 10 ng/cm² | at 300 ng/cm² |
| DIG-462 | 38 | 100 |
| DIG-463 | 63 | 100 |
| DIG-464 | 38 | 100 |
| DIG-465 | 0 | 100 |
| DIG-466 | 38 | 100 |
| DIG-467 | 38 | 88 |
| DIG-468 | 13 | 100 |
| DIG-469 | 0 | 100 |
| DIG-470 | 0 | 50 |
| DIG-471 | 0 | 0 |
| DIG-472 | 0 | 0 |
| DIG-473 | 38 | 100 |
| DIG-474 | 0 | 100 |
| BSA | 0 | 0 |

Growth inhibition of Cry1Ca core toxin (DIG-462), DIG-465, and DIG-473 protein to FAW larvae was determined to be >40% for all treatments (Table 4). Proteins were tested at full strength and diluted 5-fold with buffer (10 mM CAPS, pH 10).

TABLE 4
Percent growth inhibition of DIG-
462, DIG-465, and DIG-473 to FAW

| Protein | Dilution | % Growth Inhibition |
|---|---|---|
| DIG-462 | 1X | 43 |
| DIG-462 | 5X | 47 |
| DIG-465 | 1X | 81 |
| DIG-465 | 5X | 58 |
| DIG-473 | 1X | 56 |
| DIG-473 | 5X | 48 |
| Buffer | 1X | 0 |

DBM activity and the susceptibility of the purified protein to be digested by chymotrypsin were assessed. An unexpected and surprising finding was that DIG-473 was resistant to chymotrypsin cleavage while at the same time having the same potency against DBM as DIG-462. This is in contrast to the Cry1Ca core (DIG-462) and DIG-465 proteins, which were susceptible to chymotrypsin cleavage in vitro (Table 5).

TABLE 5
Proteins with activity against DBM (DIG-462 is the standard)
and protein resistance to cleavage by Chymotrypsin.

| | | Activity | Resistant to |
|---|---|---|---|
| DIG # | Mutation Type | on DBM | Chymotrypsin |
| 462 | truncated | ++++ | No |
| 463 | G54A | ++++ | No |
| 464 | L57M | ++++ | No |
| 465 | L57A | ++++ | No |
| 466 | V68F | ++++ | No |
| 467 | V68I | ++++ | No |
| 468 | ΔGPS | +++ | No |
| 469 | W73A | +++ | Partial |
| 473 | F596M | ++++ | Yes |
| 474 | F596A | ++++ | No |
| 482 | G54A/W73M | +++ | Yes |
| 483 | G54A/ΔGPS | +++ | Yes |
| 485 | L57A/ΔGPS | +++ | No |
| 487 | L57M/ΔGPS | +++ | No |

### Example 4

### European Corn Borer (ECB), Southwestern Corn Borer (SWCB) and Southern Armyworm (SAW) Bioassays

Bioassays were conducted in 32-well test trays. Approximately 5 mL of a 2% water-agar solution was applied to each well and the agar was allowed to solidify completely.

Plants were approximately 3 weeks old and tested at T₁ generation. Three replicates of T₁ leaf material were completed. One leaf was cut (1″×0.5″ rectangular) and placed in a single well of the tray. Each well was infested with 10 individual insect larvae (usually less than 24 hours old) of the ECB, Cry1Fa rECB or SWCB. For SAW, 5 individual insect larvae were infested per well. Seed based plants originated from B104 inbred lines and Yellow Fluorescent Protein (YFP) transformed plants served as negative controls.

The infested wells were then sealed with adhesive sheets of clear plastic, vented to allow gas exchange (C-D International, Pitman, NJ). Trays were placed in a conviron incubator and maintained at 28° C. (16:8 h light:dark, 60% RH) for 3 days, after which the total amount of damage to each leaf piece (0, 5, 10, 15, 25, 50, 75% damage, etc., up to 100%) was recorded.

There was reduced feeding damage caused by ECB and Cry1Fa resistant ECB (rECB) when the insect larvae were exposed to plants containing truncated Cry1Ca modified protein. When tested in a diet bioassay, where purified full length Cry1Ca is placed on top of an artificial insect diet and individual insects are allowed to feed on the diet containing the toxin, modified Cry1Ca is found to be inactive against ECB and rECB. However, when expressed in maize, at concentrations of >120 ng/cm², the expression of Cry1Ca in the plant provides unexpected protection against feeding damage caused by ECB and especially rECB.

TABLE 6
Bioassay results of IRDIG544.12 T₁ maize when fed to European
Corn Borer (ECB) and Cry1Fa-resistant ECB (rECB)

| | | ECB Avg. | rECB Avg. | |
|---|---|---|---|---|
| Plant Name | Description | Damage | Damage | Toxinng/cm² |
| YFP negative control | control | 100 | 88.3 | 0 |
| YFP negative control | control | 100 | 93.3 | 0 |
| YFP negative control | control | 97.7 | 98.3 | 0 |
| YFP negative control | control | 99.3 | 98 | 0 |
| YFP negative control | control | 100 | 92.7 | 0 |
| 114269[1]-021.001AJ.025 | IRDIG544.12 | 96 | 85 | 41 |
| | w/TraP12 | | | |
| 114269[1]-021.001AJ.018 | IRDIG544.12 | 94.3 | 90 | 42 |
| | w/TraP12 | | | |
| 114269[1]-021.001AJ.017 | IRDIG544.12 | 99.3 | 95 | 34 |
| | w/TraP12 | | | |
| 114269[1]-021.001AJ.023 | IRDIG544.12 | 99.3 | 50 | 36 |
| | w/TraP12 | | | |
| 114269[1]-021.001AJ.016 | IRDIG544.12 | 99.3 | 80 | 33 |
| | w/TraP12 | | | |
| 114260[1]-021.AJ001.023 | IRDIG544.12 | 66.7 | 11.7 | 210 |
| 114260[1]-021.AJ001.029 | IRDIG544.12 | 69.7 | 13.3 | 230 |
| 114260[1]-021.AJ001.021 | IRDIG544.12 | 66.7 | 16.7 | 210 |
| 114260[1]-021.AJ001.016 | IRDIG544.12 | 50 | 18.3 | 230 |
| 114260[1]-021.AJ001.019 | IRDIG544.12 | 50 | 18.3 | 210 |
| 114259[1]-009.AJ001.018 | IRDIG544.12 | 95 | 85 | 100 |
| 114259[1]-009.AJ001.022 | IRDIG544.12 | 86 | 61.7 | 130 |
| 114259[1]-009.AJ001.021 | IRDIG544.12 | 93.3 | 75 | 140 |
| 114259[1]-009.AJ001.026 | IRDIG544.12 | 80 | 95 | 67 |
| 114259[1]-009.AJ001.027 | IRDIG544.12 | 100 | 61.7 | 92 |
| 114260[1]-010.001AJ.054 | IRDIG544.12 | 68.3 | 25 | 180 |
| 114260[1]-010.001AJ.048 | IRDIG544.12 | 71.7 | 18.3 | 180 |
| 114260[1]-010.001AJ.047 | IRDIG544.12 | 66.7 | 50 | 140 |
| 114260[1]-010.001AJ.052 | IRDIG544.12 | 71.7 | 20 | 220 |
| 114260[1]-010.001AJ.046 | IRDIG544.12 | 86.7 | 16.7 | 200 |
| 114269[1]-029.001AJ.027 | IRDIG544.12 | 91 | 66.7 | 39 |
| | w/TraP12 | | | |
| 114269[1]-029.001AJ.028 | IRDIG544.12 | 88.3 | 66.7 | 30 |
| | w/TraP12 | | | |
| 114269[1]-029.001AJ.023 | IRDIG544.12 | 96.7 | 88.3 | 41 |
| | w/TraP12 | | | |
| 114269[1]-029.001AJ.026 | IRDIG544.12 | 96 | 76.7 | 37 |
| | w/TraP12 | | | |
| 114269[1]-029.001AJ.019 | IRDIG544.12 | 96.7 | 95 | 41 |
| | w/TraP12 | | | |
| 114267[1]-009.001AJ.044 | IRDIG544.12 | 95 | 70 | 36 |
| | w/TraP12 | | | |
| 114267[1]-009.001AJ.034 | IRDIG544.12 | 88.3 | 56.7 | 44 |
| | w/TraP12 | | | |
| 114267[1]-009.001AJ.032 | IRDIG544.12 | 96.7 | 86.7 | 41 |
| | w/TraP12 | | | |
| 114267[1]-009.001AJ.037 | IRDIG544.12 | 96.7 | 88.3 | 43 |
| | w/TraP12 | | | |
| 114267[1]-009.001AJ.030 | IRDIG544.12 | 91.7 | 80 | 43 |
| | w/TraP12 | | | |
| 114259[1]-006.001AJ.015 | IRDIG544.12 | 91.7 | 90 | 38 |
| 114259[1]-006.001AJ.014 | IRDIG544.12 | 98.3 | 85 | 41 |
| 114259[1]-006.001AJ.005 | IRDIG544.12 | 95 | 80 | 40 |
| 114259[1]-006.001AJ.010 | IRDIG544.12 | 81.7 | 86 | 39 |
| 114259[1]-006.001AJ.013 | IRDIG544.12 | 85 | 88.3 | 47 |
| 114270[1]-027.AJ001.029 | IRDIG544.12 | 98.7 | 96.7 | 170 |
| | w/TraP12 | | | |
| 114270[1]-027.AJ001.030 | IRDIG544.12 | 91.7 | 99.3 | 150 |
| | w/TraP12 | | | |
| 114270[1]-027.AJ001.023 | IRDIG544.12 | 93.3 | 95 | 170 |
| | w/TraP12 | | | |
| 114270[1]-027.AJ001.028 | IRDIG544.12 | 96.7 | 85 | 160 |
| | w/TraP12 | | | |
| 114270[1]-027.AJ001.027 | IRDIG544.12 | 98.3 | 86.7 | 150 |
| | w/TraP12 | | | |
| 114257[1]-016.AJ001.030 | IRDIG544.12 | 100 | 86.7 | 100 |
| 114257[1]-016.AJ001.024 | IRDIG544.12 | 68.3 | 97 | 140 |
| 114257[1]-016.AJ001.021 | IRDIG544.12 | 100 | 99.3 | 130 |
| 114257[1]-016.AJ001.027 | IRDIG544.12 | 100 | 71.7 | 130 |
| 114257[1]-016.AJ001.022 | IRDIG544.12 | 100 | 81.7 | 120 |
| 114267[1]-021.AJ001.039 | IRDIG544.12 | 91.7 | 61.7 | 230 |
| | w/TraP12 | | | |
| 114267[1]-021.AJ001.034 | IRDIG544.12 | 95 | 80 | 180 |
| | w/TraP12 | | | |
| 114267[1]-021.AJ001.044 | IRDIG544.12 | 83.3 | 75 | 210 |
| | w/TraP12 | | | |
| 114268[1]-023.AJ001.036 | IRDIG544.12 | 81 | 99 | 320 |
| | w/TraP12 | | | |
| 114268[1]-023.AJ001.041 | IRDIG544.12 | 100 | 83.3 | 410 |
| | w/TraP12 | | | |
| 114268[1]-023.AJ001.034 | IRDIG544.12 | 93.3 | 55 | 520 |
| | w/TraP12 | | | |
| 114268[1]-023.AJ001.039 | IRDIG544.12 | 91.7 | 80 | 620 |
| | w/TraP12 | | | |
| 114268[1]-023.AJ001.026 | IRDIG544.12 | 98.3 | 96 | 440 |
| | w/TraP12 | | | |
| 114268[1]-026.AJ001.053 | IRDIG544.12 | 90 | 73.3 | 390 |
| | w/TraP12 | | | |
| 114268[1]-026.AJ001.046 | IRDIG544.12 | 90 | 84.3 | 500 |
| | w/TraP12 | | | |
| 114268[1]-026.AJ001.037 | IRDIG544.12 | 99.3 | 71.7 | 320 |
| | w/TraP12 | | | |
| 114268[1]-026.AJ001.038 | IRDIG544.12 | 91.7 | 65 | 320 |
| | w/TraP12 | | | |
| 114268[1]-026.AJ001.052 | IRDIG544.12 | 97.7 | 66.7 | 360 |
| | w/TraP12 | | | |
| 114271[1]-011.001AJ.031 | IRDIG544.12 | 100 | 95 | 4 |
| | w/TraP12 | | | |
| 114271[1]-011.001AJ.042 | IRDIG544.12 | 100 | 95 | 4 |
| | w/TraP12 | | | |
| 114271[1]-011.001AJ.043 | IRDIG544.12 | 96 | 91.7 | 4 |
| | w/TraP12 | | | |
| 114271[1]-011.001AJ.047 | IRDIG544.12 | 100 | 97.7 | 3 |
| | w/TraP12 | | | |
| 114271[1]-011.001AJ.046 | IRDIG544.12 | 100 | 98 | 3 |
| | w/TraP12 | | | |
| 114270[1]-023.001AJ.050 | IRDIG544.12 | 90 | 80 | 210 |
| | w/TraP12 | | | |
| 114270[1]-023.001AJ.055 | IRDIG544.12 | 99.3 | 73.3 | 260 |
| | w/TraP12 | | | |
| 114270[1]-023.001AJ.044 | IRDIG544.12 | 100 | 92 | 250 |
| | w/TraP12 | | | |
| 114270[1]-023.001AJ.054 | IRDIG544.12 | 100 | 88.3 | 210 |
| | w/TraP12 | | | |
| 114270[1]-023.001AJ.058 | IRDIG544.12 | 100 | 82.7 | 140 |
| | w/TraP12 | | | |
| B104 | control | 100 | 100 | 0 |
| B104 | control | 100 | 100 | 0 |
| B104 | control | 100 | 100 | 0 |
| B104 | control | 100 | 99.3 | 0 |
| B104 | control | 100 | 86 | 0 |

Reduced feeding damage caused by southwestern corn borer (SWCB) and southern armyworm (SAW) was observed when the insect larvae were exposed to plants containing truncated Cry1Ca modified protein, at a range of protein expression from 140-340 ng/cm² (Table 7). The average expression was 210 ng/cm² with a standard deviation of 35.

TABLE 7
Bioassay of IRDIG544.12 T₁ maize plants when fed to southwestern
corn borer (SWCB) and southern army worm (SAW)

| | SWCB | SAW |
|---|---|---|
| Plant Name | Avg. Dmg. | Avg. Dmg. |
| 112726[1]-015.AJ001.047 | 2 | 3.0 |
| 112726[1]-015.AJ001.030 | 4 | 3.0 |
| 112726[1]-015.AJ001.019 | 1 | 1.3 |
| 112726[1]-015.AJ001.034 | 2 | 2.0 |
| YFP negative control | 98.3 | 94.3 |
| YFP negative control | 93.3 | 62.5 |
| YFP negative control | 100 | 27.5 |
| YFP negative control | 92.7 | 67.5 |
| YFP negative control | 97.7 | 30.0 |
| YFP negative control | 100 | 45.0 |
| B104 | 98.3 | 94.3 |
| B104 | 100 | 91.7 |
| B104 | 100 | 97.0 |
| B104 | 65 | 70.0 |
| B104 | 100 | 86.7 |

Field trials on corn borers were conducted at two locations: one in Indiana (IN), United States and the other in Mississippi (MS), United States. Multiple constructs and events were tested for each treatment. Cry1Ab and Cry1F served as positive controls in the ECB trials. The null served as a negative control.

To assess ECB efficacy, each plant received ten second-instar ECB larvae in the whorl of V6-V7 stage plants. In MS, Southwestern corn borer (SWCB) second-instar larvae were also artificially infested in the whorls of V9 corn (22 larvae per plant). The ECB and SWCB used were obtained from Benzon laboratory. In both ECB trials, plants were evaluated 2 weeks after infestation for foliar damage (Guthrie 1-9 scale) (Guthrie et al. 1960) where 1 is no visible injury and 9 is most leaves with long lesions (Table 8). In the MS SWCB trials, the plants were examined 4-5 days after the whorl rating for stalk damage and live insects. Data collected included number of tunnels per stalk, length of tunnels and live larvae/pupae per stalk.

TABLE 8
Corn Borer damage score criterion (whorl damage).

| Score | Criterion |
|---|---|
| 1 | No visible leaf injury or small amount of shot hole type |
| | injury on a few leaves. |
| 2 | Small amount of shot-hole type lesions on a few leaves. |
| 3 | Shot-hole injury common on several leaves. |
| 4 | Several leaves with shot-holes and elongated lesions. |
| 5 | Several leaves with elongated lesions. |
| 6 | Several leaves with elongated lesions (ca. 1 inch). |
| 7 | Long lesions common on one-half of the leaves. |
| 8 | Long lesions common on about ⅔ of the leaves. |
| 9 | Most leaves with long lesions. |

ECB Field Trials. ECB whorl damage was measured for Cry1Ca activity and showed significantly better whorl protection when compared to the null. Cry1Ca event activities were not statistically equivalent to the whorl protection provided by Cry1Ab and Cry1F.

Data generated in MS further reinforced the unexpected high level of plant protection for Cry1Ca. High feeding pressure was established in this study. Significant control was measured for Cry1Ca when compared to foliar and stalk damage on the null. Very few live insects were found surviving in the Cry1Ca stalks. Significant whorl and stalk protection was measured for Cry1Ab and Cry1F events when compared to the null.

TABLE 9
ECB Foliar Whorl Data, IN (Average Across Multiple Events)

| | | Avg. Whorl | |
|---|---|---|---|
| | | Damage Rating | Range of |
| Gene | Events | (1-9 rating) | means |
| Cry1Ca | 4 | 2.31 B | 2.00-2.65 |
| Cry1Ab | 8 | 1.00 A | 1.00 |
| Cry1F | 12 | 1.03 A | 1.00-1.20 |
| Null | 1 | 4.65 C | |

Means followed by different letters are significantly different (P ≤ 0.05).

TABLE 10
ECB Foliar Whorl and Stalk Data, MS
(Average Across Multiple Events)

| | | Avg. Whorl | | | Avg. # of |
|---|---|---|---|---|---|
| | Number | Damage | Avg. # | Avg. Length | Larvae + |
| Toxin | of | Rating | Tunnels | (cm) of | Pupae |
| Gene | Events | (1-9) | Per Stalk | Tunnels | Per Stalk |
| Cry1Ca | 3 | 2.74 B | 0.53 B | 1.43 B | 0.25 B |
| Cry1Ab | 8 | 1.66 D | 0.00 C | 0.00 C | 0.00 C |
| Cry1F | 8 | 1.85 C | 0.00 C | 0.00 C | 0.00 C |
| Null | 1 | 6.77 A | 1.93 A | 9.15 A | 1.89 A |

For all data columns, all gene events were significantly different from the null values (P < 0.05).
Within each column, means followed by different letters are significantly different (P ≤ 0.05).

In the SWCB trial, only 2 events per B.t. were evaluated. High feeding pressure was established in this study. Statistically equivalent stalk protection and number of larvae and pupae per stalk were measured for Cry1Ab, Cry1F, and Cry1Ca events.

TABLE 11
SWCB Foliar Whorl and Stalk Data, MS

| | | Avg. Whorl | | | Avg. # of |
|---|---|---|---|---|---|
| | Number | Damage | Avg. # | Avg. Length | Larvae + |
| Toxin | of | Rating | Tunnels | (cm) of | Pupae |
| Gene | Events | (1-9) | Per Stalk | Tunnels | Per Stalk |
| Cry1Ca | 2 | 1.90 C | 0.07 B | 0.20 B | 0.02 B |
| Cry1Ab | 2 | 1.91 C | 0.00 B | 0.00 B | 0.00 B |
| Cry1F | 2 | 2.18 B | 0.07 B | 0.54 B | 0.03 B |
| Null | 1 | 7.17 A | 3.53 A | 28.62 A | 3.06 A |

Within each column, means followed by different letters are significantly different (P ≤ 0.05).

The active form of Cry1Ca is composed of amino acids 29-628. The full length (1-1164), or cleaved forms (1-628 and 29-628) are active when presented to insects, since they are processed to the 29-628 form.

### Example 5

### Corn Earworm Field Trials

Field trials on corn earworm were conducted in Fowler, IN with multiple constructs and events (SEQ ID NO:31). The null served as a negative control. Each plant received five first instar larvae in the green silks of corn ears. CEW were obtained from Benzon Laboratory. Ten corn ears per plot per event were evaluated to assess the level of kernel damage in corn ears infested with CEW. All transgenic events provided significantly lower levels of kernel damage when compared to the null. There was significant suppression of larval feeding on Cry1Ca plants (Table 12).

TABLE 12
CEW Kernel Consumption Data, IN

| | | Avg. area (cm²) | % reduction in area |
|---|---|---|---|
| | | of kernel | consumed kernels |
| Gene | Entries | consumed* | (compared to null) |
| Cry1Ca | 4 | 1.72 B | 63.2 |
| Cry1F | 12 | 1.86 B | 60.3 |
| Null | 1 | 4.68 A | |

### Example 6

### Agrobacterium Transformation.

Standard cloning methods were used in the construction of binary plant transformation and expression plasmids. Agrobacterium binary plasmids which contained the cry1Ca expression cassettes were engineered using Gateway® Technology (Invitrogen, Carlsbad, CA) and used in Agrobacterium-mediated plant transformation. Restriction endonucleases were obtained from New England BioLabs (NEB; Ipswich, MA) and T4 DNA Ligase (Invitrogen) were used for DNA ligation. Gateway reactions were performed using Gateway® LR Clonase® enzyme mix (Invitrogen) Plasmid preparations were performed using the NucleoSpin® Plasmid Preparation kit or the NucleoBond® AX Xtra Midi kit (both from Macherey-Nagel), following the instructions of the manufacturers. DNA fragments were purified using the QIAquick PCR Purification Kit or the QIAEX II Gel Extraction Kit (both from Qiagen) after gel isolation.

DNA fragments comprising the nucleotide sequences that encode the insecticidal proteins, or fragments thereof, were synthesized by a commercial vendor (e.g. DNA2.0, Menlo Park, CA) and supplied as cloned fragments in standard plasmid vectors, or were obtained by standard molecular biology manipulation of other constructs containing appropriate nucleotide sequences. Unique restriction sites internal to each gene was identified and a fragment of each gene synthesized, each containing a specific deletion or insertion. The modified Cry fragments were subcloned into other Cry fragments at an appropriate restriction site to obtain a region encoding the desired full-length protein, fused proteins, or deleted variant proteins.

Electro-competent cells of Agrobacterium tumefaciens strain Z707S (a streptomycin-resistant derivative of Z707; Hepburn et al., 1985) were prepared and transformed using electroporation (Weigel and Glazebrook, 2002). After electroporation, 1 mL of YEP broth (gm/L: yeast extract, 10; peptone, 10; NaCl, 5) was added to the cuvette and the cell-YEP suspension was transferred to a 15 mL culture tube for incubation at 28° C. in a water bath with constant agitation for 4 hours. The cells were plated on YEP plus agar (25 gm/L) with spectinomycin (200 μg/mL) and streptomycin (250 μg/mL) and the plates were incubated for 2-4 days at 28° C. Well separated single colonies were selected and streaked onto fresh YEP+agar plates with spectinomycin and streptomycin as before, and incubated at 28° C. for 1-3 days.

The presence of the insecticidal protein gene insert in the binary plant transformation vector was performed by PCR analysis using vector-specific primers with template plasmid DNA prepared from selected Agrobacterium colonies. The cell pellet from a 4 mL aliquot of a 15 mL overnight culture grown in YEP with spectinomycin and streptomycin as before was extracted using Qiagen Spin Mini Preps, performed per manufacturer's instructions. Plasmid DNA from the binary vector used in the Agrobacterium electroporation transformation was included as a control. The PCR reaction was completed using Taq DNA polymerase from Invitrogen per manufacturer's instructions at 0.5× concentrations. PCR reactions were carried out in a MJ Research Peltier Thermal Cycler programmed with the following conditions: Step 1) 94° C. for 3 minutes; Step 2) 94° C. for 45 seconds; Step 3) 55° C. for 30 seconds; Step 4) 72° C. for 1 minute per kb of expected product length; Step 5) 29 times to Step 2; Step 6) 72° C. for 10 minutes. The reaction was maintained at 4° C. after cycling. The amplification products were analyzed by agarose gel electrophoresis (e.g. 0.7% to 1% agarose, w/v) and visualized by ethidium bromide staining. A colony was selected whose PCR product was identical to the plasmid control.

TABLE 13
Description of plasmids for expressing
DIG-465 and DIG-473 in maize.

| Plasmid | Description |
|---|---|
| pDAB115752 | ZmUbi1/DIG-465/ZmPer5::SCBV(MAM)v2/ |
| | AAD-1v3/ZmLip |
| pDAB115753 | ZmUbi1/DIG-473/ZmPer5::SCBV(MAM)v2/ |
| | AAD-1v3/ZmLip |
| pDAB112725 | ZmUbi1/Cry1Ca(Zm)/ZmPer5::SCBV(MAM)/ |
| | AAD-1v3./ZmLip |
| pDAB112726 | ZmUbi1/Cry1Ca(HGC)/ZmPer5::SCBV(MAM)/ |
| | AAD-1v3/ZmLip |

### Example 7

Production of DIG-465 and DIG-473 B.t. Insecticidal Proteins and Variants in Monocot Plants.

Agrobacterium-mediated transformation of maize. Seeds from a High II F1 cross (Armstrong et al., 1991) were planted into 5-gallon-pots containing a mixture of 95% Metro-Mix 360 soilless growing medium (Sun Gro Horticulture, Bellevue, WA) and 5% clay/loam soil. The plants were grown in a greenhouse using a combination of high pressure sodium and metal halide lamps with a 16:8 hour Light:Dark photoperiod. For obtaining immature F2 embryos for transformation, controlled sib-pollinations were performed. Immature embryos were isolated at 8-10 days post-pollination when embryos were approximately 1.0 to 2.0 mm in size.

Infection and co-cultivation. Maize ears were surface sterilized by scrubbing with liquid soap, immersing in 70% ethanol for 2 minutes, and then immersing in 20% commercial bleach (0.1% sodium hypochlorite) for 30 minutes before being rinsed with sterile water. A suspension Agrobacterium cells containing a superbinary vector was prepared by transferring 1-2 loops of bacteria grown on YEP solid medium containing 100 mg/L spectinomycin, 10 mg/L tetracycline, and 250 mg/L streptomycin at 28° C. for 2-3 days into 5 mL of liquid infection medium (LS Basal Medium (Linsmaier and Skoog, 1965), N6 vitamins (Chu et al., 1975), 1.5 mg/L 2,4-Dichlorophenoxyacetic acid (2,4-D), 68.5 gm/L sucrose, 36.0 gm/L glucose, 6 mM L-proline, pH 5.2) containing 100 μM acetosyringone. The solution was vortexed until a uniform suspension was achieved, and the concentration was adjusted to a final density of 200 Klett units, using a Klett-Summerson colorimeter with a purple filter. Immature embryos were isolated directly into a micro centrifuge tube containing 2 mL of the infection medium. The medium was removed and replaced with 1 mL of the Agrobacterium solution with a density of 200 Klett units, and the Agrobacterium and embryo solution was incubated for 5 minutes at room temperature and then transferred to co-cultivation medium (LS Basal Medium, N6 vitamins, 1.5 mg/L 2,4-D, 30.0 gm/L sucrose, 6 mM L-proline, 0.85 mg/L AgNO3, 100 μM Acetosyringone, 3.0 gm/L Gellan gum (PhytoTechnology Laboratories., Lenexa, KS), pH 5.8) for 5 days at 25° C. under dark conditions.

After co-cultivation, the embryos were transferred to selective medium after which transformed isolates were obtained over the course of approximately 8 weeks. For selection of maize tissues transformed with a superbinary plasmid containing a plant expressible pat or bar selectable marker gene, an LS based medium (LS Basal medium, N6 vitamins, 1.5 mg/L 2,4-D, 0.5 gm/L MES (2-(N-morpholino)ethanesulfonic acid monohydrate; PhytoTechnologies Labr.), 30.0 gm/L sucrose, 6 mM L-proline, 1.0 mg/L AgNO3, 250 mg/L cefotaxime, 2.5 gm/L Gellan gum, pH 5.7) was used with Bialaphos (Gold BioTechnology). The embryos were transferred to selection media containing 3 mg/L Bialaphos until embryogenic isolates were obtained. Recovered isolates were bulked up by transferring to fresh selection medium at 2-week intervals for regeneration and further analysis.

Regeneration and seed production. For regeneration, the cultures were transferred to “28” induction medium (MS salts and vitamins, 30 gm/L sucrose, 5 mg/L Benzylaminopurine, 0.25 mg/L 2, 4-D, 3 mg/L Bialaphos, 250 mg/L cefotaxime, 2.5 gm/L Gellan gum, pH 5.7) for 1 week under low-light conditions (14 μEm-2s-1) then 1 week under high-light conditions (approximately 89 μEm-2s-1). Tissues were subsequently transferred to “36” regeneration medium (same as induction medium except lacking plant growth regulators). When plantlets grew to 3-5 cm in length, they were transferred to glass culture tubes containing SHGA medium (Schenk and Hildebrandt salts and vitamins (1972); PhytoTechnologies Labr.), 1.0 gm/L myo-inositol, 10 gm/L sucrose and 2.0 gm/L Gellan gum, pH 5.8) to allow for further growth and development of the shoot and roots. Plants were transplanted to the same soil mixture as described earlier herein and grown to flowering in the greenhouse. Controlled pollinations for seed production were conducted.

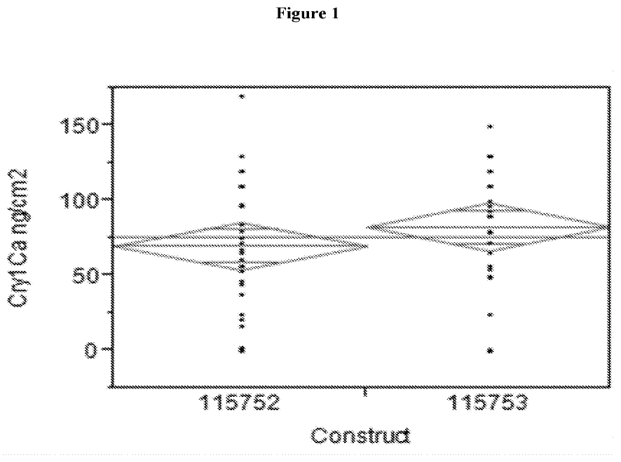

The level of expression of DIG-465 by construct 115752 and the level of expression of DIG-473 by construct 115753 is presented in FIG. 1. Both expressed similar levels of their respective proteins, at approximately 70-80 ng/cm² measured in leaves using a leaf punch to obtain the tissue sample.

SDS-PAGE of extract was taken from maize expressing the gene that encodes full length Cry1Ca protein (MR-1206) (mw 130 kDa). At least five protein products were detected by immune-blotting using a polyclonal antibody directed against Cry1Ca. The full length (130 kDa) protein, as encoded by the gene inserted into maize was detected. The other bands represent proteolytic products of this protein. A protein fragment composed of amino acid sequences 1-628, representing the core toxin was determined to have a molecular weight of 70 kDa. A 68 kDa band represented a protein composed of amino acids 29-628, where the first 28 amino acids from the N-terminus were deleted. The first three bands were functionally active against S. frugiperda and other lepidopteran insects. A fourth band represented a cleaved protein composed of amino acids 74-628 (mw 62 kDa), and a fifth band represented the Cry1Ca protein that was further processed to amino acids 74-596 (mw 59 kDa). The 62 kDa and 59 kDa bands were not functionally active against S. frugiperda and other lepidopteran insects, yet represent major protein products.

### Example 8

### Bioassay of Transgenic Maize.

Bioactivity of the DIG-465 and DIG-473 protein and variants produced in plant cells was demonstrated by methods known to those skilled in the art (see, for example Huang et al., 2006). Efficacy may be demonstrated by feeding various plant tissues or tissue pieces derived from a plant producing the DIG-465 or DIG-473 protein or variants to target insects in a controlled feeding environment. Alternatively, protein extracts may be prepared from various plant tissues derived from a plant producing the DIG-465 or DIG-473 protein or variants and incorporated in an artificial diet bioassay as previously described herein. It is to be understood that the results of such feeding assays are to be compared to similarly conducted bioassays that employ appropriate control tissues from host plants that do not produce the DIG-465 or DIG-473 protein or variants, or to other control samples.

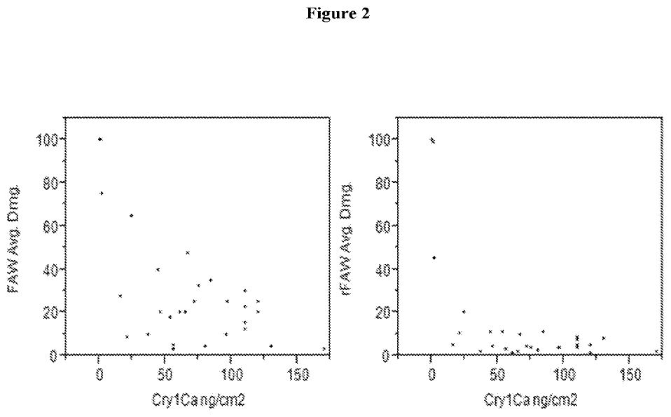

The biological activity of various events produced in maize from construct 115752 (DIG-465) were tested for preventing leaf damage caused by the feeding activity of either FAW or Cry1Fa resistant FAW (rFAW). The results show that events that expressed DIG-465 protein exhibited less feeding damage than plants not expressing the protein, and that the effect was dose dependent, with higher expression of DIG-465 resulting in less feeding damage caused by either FAW or rFAW, with the affect apparently greater against rFAW (Table 14 and FIG. 2).

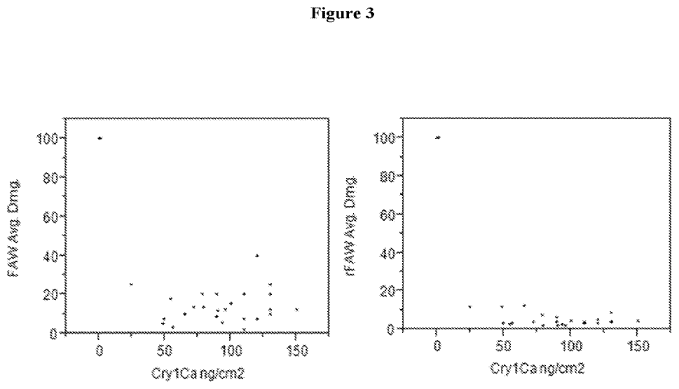

Similarly, the biological activity of various events produced in maize from construct 115753 (DIG-473) were tested for preventing leaf damage caused by the feeding activity of either FAW or Cry1Fa resistant FAW (rFAW). The results show that events that expressed DIG-473 protein exhibited less feeding damage than plants not expressing the protein, and that the effect was dose dependent, with higher expression of DIG-473 resulting in less feeding damage caused by either FAW or rFAW, with the affect apparently greater against rFAW (Table 14 and FIG. 3).

TABLE 14
FAW bioassay data when exposed to
DIG-465, DIG-473, or controls

| | | FAW | rFAW | Accumulated |
|---|---|---|---|---|
| | | Avg. | Avg. | toxin |
| Plant Name | DIG # | Dmg. | Dmg. | (ng/cm²) |
| 115752[1]-001.001 | DIG-465 | 65 | 20 | 24 |
| 115752[1]-002.001 | DIG-465 | 20 | 4.375 | 46 |
| 115752[1]-003.001 | DIG-465 | 32.5 | 3.75 | 75 |
| 115752[1]-004.001 | DIG-465 | 100 | 98.75 | 0.7 |
| 115752[1]-005.001 | DIG-465 | 30 | 8.75 | 110 |
| 115752[1]-006.001 | DIG-465 | 40 | 11.25 | 44 |
| 115752[1]-007.001 | DIG-465 | 22.5 | 5 | 110 |
| 115752[1]-009.001 | DIG-465 | 3.125 | 3.125 | 56 |
| 115752[1]-010.001 | DIG-465 | 17.5 | 11.25 | 53 |
| 115752[1]-011.001 | DIG-465 | 3.125 | 1.75 | 170 |
| 115752[1]-012.001 | DIG-465 | 27.5 | 5 | 16 |
| 115752[1]-013.001 | DIG-465 | 20 | 1 | 61 |
| 115752[1]-014.001 | DIG-465 | 25 | 3.75 | 97 |
| 115752[1]-016.001 | DIG-465 | 15 | 3.75 | 110 |
| 115752[1]-017.001 | DIG-465 | 75 | 45 | 2 |
| 115752[1]-018.001 | DIG-465 | 25 | 4.375 | 72 |
| 115752[1]-019.001 | DIG-465 | 10 | 2.125 | 37 |
| 115752[1]-020.001 | DIG-465 | 25 | 1 | 120 |
| 115752[1]-021.001 | DIG-465 | 20 | 1.75 | 65 |
| 115752[1]-022.001 | DIG-465 | 12.5 | 7.5 | 110 |
| 115752[1]-023.001 | DIG-465 | 35 | 11.25 | 84 |
| 115752[1]-024.001 | DIG-465 | 47.5 | 10 | 67 |
| 115752[1]-025.001 | DIG-465 | 20 | 5 | 120 |
| 115752[1]-026.001 | DIG-465 | 4.375 | 8 | 130.00 |
| 115752[1]-027.001 | DIG-465 | 5 | 3.125 | 56 |
| 115752[1]-028.001 | DIG-465 | 10 | 3.75 | 96 |
| 115752[1]-029.001 | DIG-465 | 4.375 | 2.5 | 80 |
| 115752[1]-030.001 | DIG-465 | 8.75 | 10.625 | 21 |
| 115752[1]-031.001 | DIG-465 | 100 | 100 | 0 |
| 115753[1]-001.001 | DIG-473 | 100 | 100 | 0 |
| 115753[1]-002.001 | DIG-473 | 12.5 | 8.75 | 130 |
| 115753[1]-003.001 | DIG-473 | 20 | 7.5 | 78 |
| 115753[1]-004.001 | DIG-473 | 20 | 3.125 | 110 |
| 115753[1]-005.001 | DIG-473 | 25 | 11.875 | 24 |
| 115753[1]-006.001 | DIG-473 | 12.5 | 1.75 | 96 |
| 115753[1]-007.001 | DIG-473 | 8.75 | 3.75 | 89 |
| 115753[1]-008.001 | DIG-473 | 25 | 3.375 | 130 |
| 115753[1]-010.001 | DIG-473 | 20 | 6.25 | 89 |
| 115753[1]-011.001 | DIG-473 | 15 | 4.375 | 100 |
| 115753[1]-012.001 | DIG-473 | 13.75 | 2.125 | 79 |
| 115753[1]-013.001 | DIG-473 | 17.5 | 2.75 | 54 |
| 115753[1]-014.001 | DIG-473 | 10 | 12.5 | 65 |
| 115753[1]-015.001 | DIG-473 | 20 | 3.75 | 130 |
| 115753[1]-016.001 | DIG-473 | 7.5 | 3.125 | 49 |
| 115753[1]-017.001 | DIG-473 | 2.125 | 3.125 | 110 |
| 115753[1]-018.001 | DIG-473 | 100 | 100 | 0.7 |
| 115753[1]-019.001 | DIG-473 | 100 | 100 | 0 |
| 115753[1]-020.001 | DIG-473 | 11.875 | 1.75 | 90 |
| 115753[1]-021.001 | DIG-473 | 10 | 3.75 | 130 |
| 115753[1]-022.001 | DIG-473 | 40 | 5 | 120 |
| 115753[1]-023.001 | DIG-473 | 7.5 | 3.75 | 110 |
| 115753[1]-025.001 | DIG-473 | 13.75 | 3.375 | 72 |
| 115753[1]-026.001 | DIG-473 | 12.5 | 4.375 | 150 |
| 115753[1]-027.001 | DIG-473 | 5.625 | 2.5 | 93 |
| 115753[1]-028.001 | DIG-473 | 5 | 11.875 | 48 |
| 115753[1]-029.001 | DIG-473 | 3.125 | 3.125 | 56 |
| 115753[1]-030.001 | DIG-473 | 7.5 | 3.125 | 120.00 |
| B104 | | 100 | 100 | N/A |
| B104 | | 100 | 100 | N/A |
| B104 | | 100 | 100 | N/A |
| YFP negative control | | 100 | 100 | N/A |
| YFP negative control | | 100 | 100 | N/A |
| YFP negative control | | 100 | 100 | N/A |
| B104 | | 100 | 100 | N/A |
| B104 | | 100 | 100 | N/A |
| B104 | | 100 | 100 | N/A |
| YFP negative control | | 100 | 100 | N/A |
| YFP negative control | | 100 | 100 | N/A |
| YFP negative control | | 100 | 100 | N/A |

### Example 9

### Production of Bt Insecticidal Proteins and Variants in Dicot Plants.

Arabidopsis Transformation. Arabidopsis thaliana Col-01 was transformed using the floral dip method (Weigel and Glazebrook, 2002). The selected Agrobacterium colony was used to inoculate 1 mL to 15 mL cultures of YEP broth containing appropriate antibiotics for selection. The culture was incubated overnight at 28° C. with constant agitation at 220 rpm. Each culture was used to inoculate two 500 mL cultures of YEP broth containing appropriate antibiotics for selection and the new cultures were incubated overnight at 28° C. with constant agitation. The cells were centrifuged at approximately 8700× g for 10 minutes at room temperature, and the resulting supernatant was discarded. The cell pellet was gently resuspended in 500 mL of infiltration media containing: ½× Murashige and Skoog salts (Sigma-Aldrich)/Gamborg's B5 vitamins (Gold BioTechnology, St. Louis, MO), 10% (w/v) sucrose, 0.044 μM benzylaminopurine (10 μL/L of 1 mg/mL stock in DMSO) and 300 μL/L Silwet L-77. Plants approximately 1 month old were dipped into the media for 15 seconds, with care taken to assure submergence of the newest inflorescence. The plants were then laid on their sides and covered (transparent or opaque) for 24 hours, washed with water, and placed upright. The plants were grown at 22° C., with a 16:8 light:dark photoperiod. Approximately 4 weeks after dipping, the seeds were harvested.

Arabidopsis Growth and Selection Freshly harvested T₁ seed was allowed to dry for at least 7 days at room temperature in the presence of desiccant. Seed was suspended in a 0.1% agar/water (Sigma-Aldrich) solution and then stratified at 4° C. for 2 days. To prepare for planting, Sunshine Mix LP5 (Sun Gro Horticulture Inc., Bellevue, WA) in 10.5 inch×21 inch germination trays (T.O. Plastics Inc., Clearwater, MN) was covered with fine vermiculite, sub-irrigated with Hoagland's solution (Hoagland and Arnon, 1950) until wet, then allowed to drain for 24 hours. Stratified seed was sown onto the vermiculite and covered with humidity domes (KORD Products, Bramalea, Ontario, Canada) for 7 days. Seeds were germinated and plants were grown in a Conviron (Models CMP4030 or CMP3244; Controlled Environments Limited, Winnipeg, Manitoba, Canada) under long day conditions (16:8 light:dark photoperiod) at a light intensity of 120-150 μmol/m²sec under constant temperature (22° C.) and humidity (40-50%). Plants were initially watered with Hoagland's solution and subsequently with deionized water to keep the soil moist but not wet.

The domes were removed 5-6 days post sowing and plants were sprayed with a chemical selection agent to kill plants germinated from nontransformed seeds. For example, if the plant expressible selectable marker gene provided by the binary plant transformation vector was a pat or bar gene (Wehrmann et al., 1996), transformed plants may be selected by spraying with a 1000× solution of Finale (5.78% glufosinate ammonium, Farnam Companies Inc., Phoenix, AZ.). Two subsequent sprays were performed at 5-7 day intervals. Survivors (plants actively growing) were identified 7-10 days after the final spraying and were transplanted into pots prepared with Sunshine Mix LP5. Transplanted plants were covered with a humidity dome for 3-4 days and placed in a Conviron incubator under the above-mentioned growth conditions.

Those skilled in the art of dicot plant transformation will understand that other methods of selection of transformed plants are available when other plant expressible selectable marker genes (e.g. herbicide tolerance genes) are used.

### Example 10

### Transgenic Glycine max Comprising DIG Protein

Ten to 20 transgenic T₀ Glycine max plants harboring expression vectors for nucleic acids comprising Cry1Ca protein were generated as is known in the art, including for example by Agrobacterium-mediated transformation. Mature soybean (Glycine max) seeds were sterilized overnight with chlorine gas for sixteen hours. Following sterilization with chlorine gas, the seeds were placed in an open container in a LAMINAR™ flow hood to dispel the chlorine gas. Next, the sterilized seeds were imbibed with sterile H₂O for sixteen hours in the dark using a black box at 24° C.

Preparation of split-seed soybeans. The split soybean seed comprising a portion of an embryonic axis protocol required preparation of soybean seed material which was cut longitudinally, using a #10 blade affixed to a scalpel, along the hilum of the seed to separate and remove the seed coat, and to split the seed into two cotyledon sections. Careful attention was made to partially remove the embryonic axis, wherein about ½-⅓ of the embryo axis remained attached to the nodal end of the cotyledon.

Inoculation. The split soybean seeds comprising a partial portion of the embryonic axis were then immersed for about 30 minutes in a solution of Agrobacterium tumefaciens (e.g., strain EHA 101 or EHA 105) containing binary plasmid comprising DIG protein. The Agrobacterium tumefaciens solution was diluted to a final concentration of λ=0.6 OD₆₅₀ before immersing the cotyledons comprising the embryo axis.

Co-cultivation. Following inoculation, the split soybean seed was allowed to co-cultivate with the Agrobacterium tumefaciens strain for 5 days on co-cultivation medium (Wang, Kan. Agrobacterium Protocols. 2. 1. New Jersey: Humana Press, 2006. Print.) in a Petri dish covered with a piece of filter paper.

Shoot induction. After 5 days of co-cultivation, the split soybean seeds were washed in liquid Shoot Induction (SI) media consisting of B5 salts, B5 vitamins, 28 mg/L Ferrous, 38 mg/L Na₂EDTA, 30 g/L sucrose, 0.6 g/L MES, 1.11 mg/L BAP, 100 mg/L TIMENTIN™, 200 mg/L cefotaxime, and 50 mg/L vancomycin (pH 5.7). The split soybean seeds were then cultured on Shoot Induction I (SI I) medium consisting of B5 salts, B5 vitamins, 7 g/L Noble agar, 28 mg/L Ferrous, 38 mg/L Na₂EDTA, 30 g/L sucrose, 0.6 g/L MES, 1.11 mg/L BAP, 50 mg/L TIMENTIN™, 200 mg/L cefotaxime, 50 mg/L vancomycin (pH 5.7), with the flat side of the cotyledon facing up and the nodal end of the cotyledon imbedded into the medium. After 2 weeks of culture, the explants from the transformed split soybean seed were transferred to the Shoot Induction II (SI II) medium containing SI I medium supplemented with 6 mg/L glufosinate (LIBERTY®).

Shoot elongation. After 2 weeks of culture on SI II medium, the cotyledons were removed from the explants and a flush shoot pad containing the embryonic axis were excised by making a cut at the base of the cotyledon. The isolated shoot pad from the cotyledon was transferred to Shoot Elongation (SE) medium. The SE medium consisted of MS salts, 28 mg/L Ferrous, 38 mg/L Na₂EDTA, 30 g/L sucrose and 0.6 g/L MES, 50 mg/L asparagine, 100 mg/L L-pyroglutamic acid, 0.1 mg/L IAA, 0.5 mg/L GA3, 1 mg/L zeatin riboside, 50 mg/L TIMENTIN™, 200 mg/L cefotaxime, 50 mg/L vancomycin, 6 mg/L glufosinate, 7 g/L Noble agar, (pH 5.7). The cultures were transferred to fresh SE medium every 2 weeks. The cultures were grown in a CONVIRON™ growth chamber at 24° C. with an 18 h photoperiod at a light intensity of 80-90 μmol/m² sec.

Rooting. Elongated shoots which developed from the cotyledon shoot pad were isolated by cutting the elongated shoot at the base of the cotyledon shoot pad, and dipping the elongated shoot in 1 mg/L IBA (Indole 3-butyric acid) for 1-3 minutes to promote rooting. Next, the elongated shoots were transferred to rooting medium (MS salts, B5 vitamins, 28 mg/L Ferrous, 38 mg/L Na₂EDTA, 20 g/L sucrose and 0.59 g/L MES, 50 mg/L asparagine, 100 mg/L L-pyroglutamic acid 7 g/L Noble agar, pH 5.6) in phyta trays.

Cultivation. Following culture in a CONVIRON™ growth chamber at 24° C., 18 h photoperiod, for 1-2 weeks, the shoots which had developed roots were transferred to a soil mix in a covered sundae cup and placed in a CONVIRON™ growth chamber (models CMP4030 and CMP3244, Controlled Environments Limited, Winnipeg, Manitoba, Canada) under long day conditions (16 hours light/8 hours dark) at a light intensity of 120-150 μmol/m²sec under constant temperature (22° C.) and humidity (40-50%) for acclimatization of plantlets. The rooted plantlets were acclimated in sundae cups for several weeks before they were transferred to the greenhouse for further acclimatization and establishment of robust transgenic soybean plants.

Development and morphological characteristics of transgenic lines were compared with nontransformed plants. Plant root, shoot, foliage and reproduction characteristics were compared. There were no observable difference in root length and growth patterns of transgenic and nontransformed plants. Plant shoot characteristics such as height, leaf numbers and sizes, time of flowering, floral size and appearance were similar. In general, there were no observable morphological differences between transgenic lines and those without expression of DIG proteins when cultured in vitro and in soil in the glasshouse.

### Example 11

### Transformation of Additional Crop Species

Cotton is transformed with B.t. proteins (with or without a chloroplast transit peptide) to provide control of lepidopteran insects by utilizing a method known to those of skill in the art, for example, substantially the same techniques previously described in EXAMPLE 9 of U.S. Pat. No. 7,838,733, or Example 12 of PCT International Patent Publication No. WO 2007/053482.

It should be understood that the examples and embodiments described herein are for illustrative purposes only and that various modifications or changes in light thereof will be suggested to persons skilled in the art and are to be included within the spirit and purview of this application and the scope of the appended claims. With the teachings provided herein, one skilled in the art could readily produce and use the various toxins and polynucleotide sequences described herein.

1-17. (canceled)

18. A nucleic acid sequence chosen from the group consisting of SEQ ID NO:1, SEQ ID NO:3, SEQ ID NO:5, SEQ ID NO:7, SEQ ID NO:9, SEQ ID NO: 11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, and SEQ ID NO:39

19. A transgenic plant, plant part, or seed comprising a nucleic acid sequence of claim 18.

20. The transgenic plant, plant part, or seed of claim 19 chosen from the group consisting of maize, sunflower, soybean, cotton, canola, rice, sorghum, wheat, barley, vegetables, ornamentals, peppers, sugar beets, fruit, and turf grass.

21. The transgenic plant, plant part, or seed of claim 19 chosen from the group consisting of maize, soybean and cotton.

22. The transgenic plant, plant part, or seed of claim 19 that is maize.

23. The transgenic plant, plant part, or seed of claim 19 that is soybean.

24. The transgenic plant, plant part, or seed of claim 19 that is cotton.

25. A method of controlling plant insect pests which comprises growing transgenic plants that comprise a nucleic acid sequence of claim 18, and allowing susceptible pests to feed on said transgenic plants.

26. A method of controlling plant insect pests that have developed resistance to other Cry toxins which comprises growing transgenic plants that comprise a nucleic acid sequence of claim 18, and allowing susceptible pests to feed on said transgenic plants.
